(19) United States
(12) Patent Application Publication
(10) Pub. No.: US 2010/0280336 A1
(43) Pub. Date: Nov. 4, 2010

(54) ANXIETY DISORDER MONITORING

(75) Inventors: Jonathon E. Giftakis, Maple Grove, MN (US); Mark T. Rise, Monticello, MN (US); Paul H. Stypulkowski, North Oaks, MN (US)

Correspondence Address: SHUMAKER & SIEFFERT , P.A, 1625 RADIO DRIVE , SUITE 300, WOODBURY, MN, 55125, US

(73) Assignee: Medtronic, Inc.

(21) Appl. No.: 12/751,537

(22) Filed: Mar. 31, 2010

Related U.S. Application Data
(60) Provisional application No. 61/174,464, filed on Apr. 30, 2009.

(51) Int. Cl. A61B 5/00

(52) U.S. Cl. 600/301

(57) ABSTRACT

An anxiety episode may be identified as being an anxiety event that is attributable to an anxiety disorder of a patient based on the patient activity associated with the anxiety episode. The patient activity may include, for example, patient motion, patient posture or voice activity. Detection of the activity component during an anxiety episode can help distinguish between a general anxiety state and an anxiety event that differs from the general anxiety state. Examples of anxiety events include, for example, an occurrence of a compulsion or a panic attack. The detected anxiety events can be used to evaluate an anxiety disorder of a patient, evaluate therapy programs implemented by a medical device to treat the anxiety disorder, or control therapy delivery. In some examples, a mood state transition is detected based on patient activity information and therapy delivery is controlled based on the detection of the mood state transition.

This application claims the benefit of U.S. Provisional Application No. 61/174,464 by Giftakis et al., entitled, “ANXIETY DISORDER MONITORING” and filed on Apr. 30, 2009, the entire content of which is incorporated herein by reference.

### TECHNICAL FIELD

The disclosure relates to patient monitoring, and, more particularly, detecting a patient event related to a patient condition.

### BACKGROUND

Implantable medical devices, such as electrical stimulators or therapeutic agent delivery devices, may be used in different therapeutic applications, such as deep brain stimulation (DBS), spinal cord stimulation (SCS), pelvic stimulation, gastric stimulation, peripheral nerve stimulation, functional electrical stimulation or delivery of pharmaceutical agent, insulin, pain relieving agent or anti-inflammatory agent to a target tissue site within a patient. A medical device may be used to deliver therapy to a patient to treat a variety of symptoms or patient conditions such as chronic pain, tremor, Parkinson's disease, other types of movement disorders, seizure disorders (e.g., epilepsy), urinary or fecal incontinence, sexual dysfunction, obesity, psychiatric disorders, gastroparesis or diabetes. In some therapy systems, an implantable electrical stimulator delivers electrical therapy to a target tissue site within a patient with the aid of one or more electrodes, which may be deployed by medical leads. In addition to or instead of electrical stimulation therapy, a medical device may deliver a therapeutic agent to a target tissue site within a patient with the aid of one or more fluid delivery elements, such as a catheter or a therapeutic agent eluting patch.

### SUMMARY

In general, the disclosure is directed to monitoring an anxiety disorder of a patient. In some examples, patient activity (e.g., physical motion, posture or voice activity) is monitored in order to identify an anxiety event that is attributable to the anxiety disorder. The anxiety event occurs during an anxiety episode, during which an anxiety level of the patient is relatively high compared to, e.g., a baseline level, and is characterized by the occurrence of a specific patient activity, which can be, for example, a motor activity or voice activity. The motor activity can be a tic, compulsive behavior, or another physical patient motion during the anxiety episode. Accordingly, detection of the specific patient activity (referred to as an activity component) during the anxiety episode may indicate an occurrence of an anxiety event. In contrast, an anxiety episode for which no associated activity component is detected may be a benign anxiety episode in the sense that the anxiety state is not directly attributable to the anxiety disorder of the patient. Detection of the activity component during an anxiety episode can help distinguish between general anxiety and an anxiety event that differs from the general anxiety (e.g., compulsive behavior accompanied by anxiety).

In addition, the disclosure is directed to detecting a mood state transition based on patient activity information, where the activity information may be indicative of an activity level, a posture state, and/or voice activity of the patient. In some examples, therapy delivery to the patient is controlled based on the detection of the mood state transition.

In one aspect, the disclosure is directed to a method comprising detecting, with a processor, an anxiety episode of a patient based on a physiological parameter of the patient, and determining, with the processor, whether the anxiety episode is an anxiety event attributable to an anxiety disorder of the patient based on a signal generated by a patient activity sensor. In some examples, the anxiety disorder can comprise at least one of obsessive compulsive disorder, post-traumatic stress disorder, a panic disorder, or Tourette's syndrome. Other anxiety disorders are contemplated.

In another aspect, the disclosure is directed to a method comprising receiving a first signal indicative of an anxiety state of a patient, receiving a second signal indicative of at least one of motion, posture state or voice activity of the patient, and identifying, with a processor, an occurrence of an anxiety event during an anxiety state based on the first and second signals.

In another aspect, the disclosure is directed to a system comprising a first sensing module that generates a first signal indicative of a physiological parameter of a patient, a second sensing module that generates a second signal indicative of activity of the patient, and a processor that detects an anxiety episode of the patient based on the first signal and determines whether the anxiety episode is an anxiety event attributable to an anxiety disorder of the patient based on the second signal.

In another aspect, the disclosure is directed to a system comprising a first sensing module that generates a first signal indicative of an anxiety episode of a patient, a second sensing module that generates a second signal indicative of at least one of motion, posture state or voice activity of the patient, and a processor that identifies an occurrence of an anxiety event based on the first and second signals.

In another aspect, the disclosure is directed to a system comprising means for detecting an anxiety episode of a patient based on a physiological parameter of the patient, means for receiving a signal generated by a patient activity sensor, and means for determining whether the anxiety episode is an anxiety event attributable to an anxiety disorder of the patient based on the signal.

In another aspect, the disclosure is directed to a system comprising means for receiving a first signal indicative of an anxiety state of a patient, means for receiving a second signal indicative of at least one of motion, posture state or voice activity of the patient, and means for identifying an occurrence of an anxiety event during an anxiety state based on the first and second signals.

In another aspect, the disclosure is directed to a computer-readable storage medium comprising instructions. The instructions cause a programmable processor to detect an anxiety episode of a patient based on a physiological parameter of the patient, and determine whether the anxiety episode is an anxiety event attributable to an anxiety disorder of the patient based on a signal generated by a patient activity sensor.

In another aspect, the disclosure is directed to a computer-readable storage medium comprising instructions. The instructions cause a programmable processor to receive a first signal indicative of an anxiety state of a patient, receive a second signal indicative of at least one of motion, posture state or voice activity of the patient, and identify an occurrence of an anxiety event during an anxiety state based on the first and second signals.

In another aspect, the disclosure is directed to a method comprising delivering therapy to a patient to manage a first mood state of a patient, detecting a transition from the first mood state to a second mood state based on patient activity information, and adjusting therapy delivery to the patient based on the transition.

In another aspect, the disclosure is directed to a system comprising a sensing module that generates a signal indicative of activity of a patient, a medical device that delivers therapy to the patient to manage a first mood state of the patient, and a processor that detects a transition from the first mood state to a second mood state based on patient activity information and controls the medical device to adjust the therapy delivery to the patient based on the transition.

In another aspect, the disclosure is directed to a system comprising means for delivering therapy to a patient to manage a first mood state of a patient, means for detecting a transition from the first mood state to a second mood state based on patient activity information, and means for adjusting therapy delivery to the patient based on the transition.

In another aspect, the disclosure is directed to a computer-readable storage medium comprising instructions. The instructions cause a programmable processor to control a medical device to deliver therapy to a patient to manage a first mood state of a patient, detect a transition from the first mood state to a second mood state based on patient activity information, and adjust therapy delivery to the patient by the medical device based on the transition.

In another aspect, the disclosure is directed to an article of manufacture comprising a computer-readable storage medium comprising instructions. The instructions cause a programmable processor to perform any part of the techniques described herein. The instructions may be, for example, software instructions, such as those used to define a software or computer program. The computer-readable medium may be a computer-readable storage medium such as a storage device (e.g., a disk drive, or an optical drive), memory (e.g., a Flash memory, random access memory or RAM) or any other type of volatile or non-volatile memory that stores instructions (e.g., in the form of a computer program or other executable) to cause a programmable processor to perform the techniques described herein.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example deep brain stimulation (DBS) system that includes at least one sensor that generate a signal indicative of patient activity associated with an anxiety event.

FIG. 2 is functional block diagram illustrating components of an example medical device.

FIG. 3 is a functional block diagram illustrating components of an example medical device programmer.

FIG. 4 is a flow diagram of an example technique for monitoring an anxiety disorder of a patient by identifying anxiety events based on monitored patient activity.

FIGS. 5-7 are conceptual illustrations of a bioelectrical brain signal and a patient activity signal that indicate the occurrence of an anxiety event.

FIG. 8 is a conceptual illustration of a data structure that presents a list of example detected anxiety episodes and associated anxiety metrics.

FIG. 9 is a schematic illustration of a clinician programmer, which includes a display presenting a graphical user interface (GUI) evaluating anxiety episodes of a patient.

FIG. 10 is a flow diagram of an example technique for associating a detected anxiety event with a therapy program.

FIG. 11 is a schematic illustration of a clinician programmer, which includes a display presenting a GUI listing a plurality of therapy programs and respective evaluation metrics.

FIG. 12 is a flow diagram of an example technique for controlling therapy delivery to a patient based on the detection of an anxiety event that is associated with a motor component.

FIG. 13 is a flow diagram of an example technique for determining a brain signal characteristic indicative of an anxiety event.

FIG. 14 is a flow diagram of an example technique for training a support vector machine algorithm to respond to future patient parameter signal inputs and classify the patient parameter signal inputs as being representative of a first patient state or a second patient state.

FIGS. 15A and 15B are conceptual illustrations of feature spaces with respective boundaries separate feature vectors into first and second classes.

FIG. 16 is a flow diagram of an example technique for determining a patient state with a support vector machine algorithm.

FIG. 17 is a flow diagram of an example technique for controlling therapy delivery to a patient based on a detected mood state transition.

### DETAILED DESCRIPTION

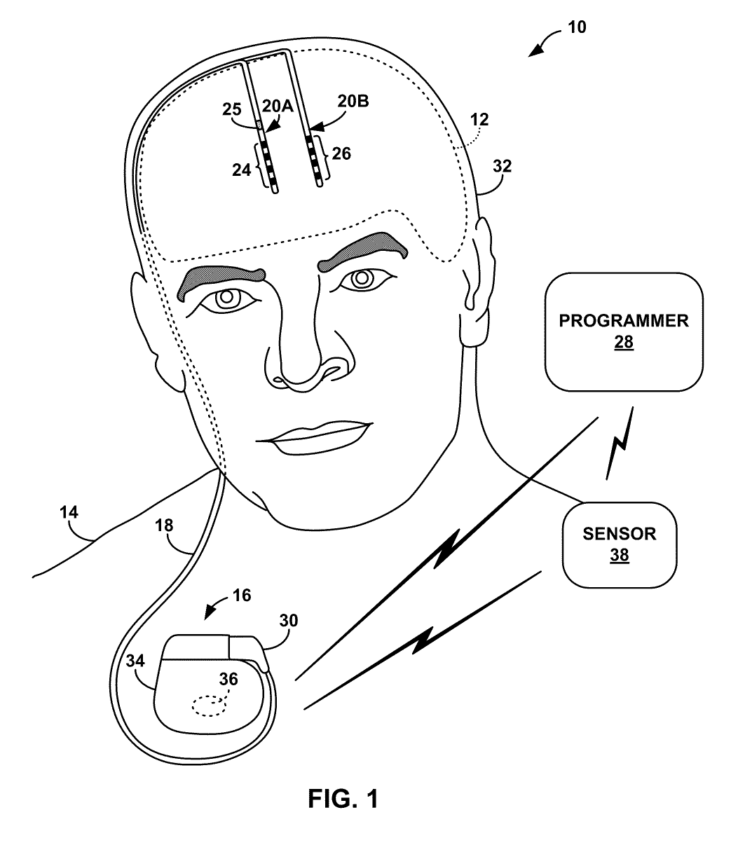

FIG. 1 is a conceptual diagram illustrating an example therapy system 10 that is implanted proximate to brain 12 of patient 14 in order to help manage a patient condition, such as a psychiatric disorder. Examples of psychiatric disorders that therapy system 10 may be useful for managing include major depressive disorder (MDD), bipolar disorder, anxiety disorders (e.g., post traumatic stress disorder (PTSD), obsessive-compulsive disorder (OCD), panic disorder), dysthymic disorder, addictions or substance abuse disorders, or psychotic disorders (e.g., schizophrenia). While patient 14 is generally referred to as a human patient, other mammalian or non-mammalian patients are also contemplated.

Therapy system 10 includes implantable medical device (IMD) 16, lead extension 18, leads 20A and 20B with respective sets of electrodes 24, 26, and medical device programmer 28. IMD 16 includes a therapy module that delivers electrical stimulation therapy to one or more regions of brain 12 via leads 20A and 20B (collectively referred to as “leads 20”). In the example shown in FIG. 1, therapy system 10 may be referred to as a deep brain stimulation (DBS) system because IMD 16 provides electrical stimulation therapy directly to tissue within brain 12, e.g., a tissue site under the dura mater of brain 12. In other examples, leads 20 may be positioned to deliver therapy to a surface of brain 12 (e.g., the cortical surface of brain 12) or to a peripheral or cranial nerve (e.g., the vagus nerve).

In the example shown in FIG. 1, IMD 16 may be implanted subcutaneous cavity over the chest of patient 14. In other examples, IMD 16 may be implanted within other regions of patient 14, such as a subcutaneous pocket in the abdomen of patient 14 or proximate the cranium of patient 14. Implanted lead extension 18 is coupled to IMD 16 via connector block 30, which may include, for example, electrical contacts that electrically couple to respective electrical contacts on lead extension 18. The electrical contacts electrically couple the electrodes carried by leads 20 to IMD 16. Lead extension 18 traverses from the implant site of IMD 16 along the neck of patient 14 and through cranium 32 of patient 14 to access brain 12.

Leads 20 may be positioned to deliver electrical stimulation to one or more target tissue sites within brain 12 to manage patient symptoms associated with a psychiatric disorder of patient 14. Leads 20 may be implanted to position electrodes 24, 26 at desired locations of brain 12 through respective holes in cranium 32. Leads 20 may be placed at any location within brain 12 such that electrodes 24, 26 are capable of providing electrical stimulation to target tissue sites within brain 12 during treatment. In the example shown in FIG. 1, leads 20 are implanted within the right and left hemispheres, respectively, of brain 12 in order to deliver electrical stimulation to one or more regions of brain 12, which may be selected based on many factors, such as the type of patient condition for which therapy system 10 is implemented to manage. Although two leads 20 are shown in FIG. 1, in other examples, therapy system 10 may include any suitable number of leads, such as one or more than two.

Different neurological or psychiatric disorders may be associated with activity in one or more of the regions of brain 12, which may differ between patients. For example, in the case of MDD, bipolar disorder, OCD or other anxiety disorders, leads 20 may be implanted to deliver electrical stimulation to the anterior limb of the internal capsule of brain 12, only the ventral portion of the anterior limb of the internal capsule and ventral striatum (also referred to as a VC/VS), the subgenual component of the cingulate cortex (Brodmann area 25), anterior cingulate cortex (Brodmann areas 32 and 24), various parts of the prefrontal cortex, including the dorsal lateral and medial pre-frontal cortex (PFC) (e.g., Brodmann area 9 and 46), ventromedial prefrontal cortex (e.g., Brodmann area 10), the lateral and medial orbitofrontal cortex (e.g., Brodmann area 11), nucleus accumbens, the dorsal medial thalamus, intralaminar thalamic nuclei, amygdala, hippocampus, the lateral hypothalamus, the Locus ceruleus, the dorsal raphe nucleus, ventral tegmentum, the substantia nigra, subthalamic nucleus, the inferior thalamic peduncle, the dorsal medial nucleus of the thalamus, the habenula, the vagus nerve or any combination thereof

Although leads 20 are shown in FIG. 1 as being coupled to a common lead extension 18, in other examples, leads 20 may be coupled to IMD 16 via separate lead extensions or directly coupled to connector block 30 of IMD 16. Leads 20 may deliver electrical stimulation to treat any number of neurological disorders or diseases in addition to psychiatric disorders, such as movement disorders or seizure disorders. Examples of movement disorders include a reduction in muscle control, motion impairment or other movement problems, such as rigidity, bradykinesia, rhythmic hyperkinesia, nonrhythmic hyperkinesia, dystonia, tremor, and akinesia. Movement disorders may be associated with patient disease states, such as Parkinson's disease or Huntington's disease. An example seizure disorder includes epilepsy.

Leads 20 may be implanted within a desired location of brain 12 via any suitable technique, such as through respective burr holes in a skull of patient 14 or through a common burr hole in the cranium. Electrical stimulation generated from the signal generator (not shown) of IMD 16 may help prevent the onset of events associated with the patient's psychiatric disorder or mitigate symptoms of the psychiatric disorder. For example, electrical stimulation therapy delivered by IMD 16 to a target tissue site within brain 12 may help prevent an anxiety event (e.g., characterized by the undertaking of a specific patient activity during an anxiety episode) if patient 14 has OCD, or help prevent or minimize the duration and/or severity of a PTSD event of patient 14. The exact therapy parameter values of the stimulation therapy, such as the amplitude or magnitude of the stimulation signals, the duration of each signal, the waveform of the stimuli (e.g., rectangular, sinusoidal or ramped signals), the frequency of the signals, and the like, may be specific for the particular target stimulation site (e.g., the region of the brain) involved as well as the particular patient and patient condition.

In the case of stimulation pulses, the stimulation therapy may be characterized by selected pulse parameters, such as pulse amplitude, pulse rate, and pulse width. In addition, if different electrodes are available for delivery of stimulation, the therapy may be further characterized by different electrode combinations, which can include selected electrodes and their respective polarities. Stimulation may be delivered between electrodes of the same lead 24 or 26 or between electrodes of both leads 24, 26. Known techniques for determining useful stimulation parameters for patient 14 may be employed. In one example, electrodes of leads 20 are positioned to deliver stimulation therapy to an anterior limb of the internal capsule of brain 12 in order to manage symptoms of an anxiety disorder of patient 14, and stimulation therapy is delivered via a selected combination of the electrodes to the anterior limb of the internal capsule with electrical stimulation including a frequency of about 2 hertz (Hz) to about 2000 Hz, a voltage amplitude of about 0.5 volts (V) to about 50 V, and a pulse width of about 60 microseconds (μs) to about 4 milliseconds (ms). However, other examples may implement stimulation therapy including other stimulation parameters.

The electrodes of leads 20 are shown as ring electrodes. Ring electrodes may be relatively easy to program and are typically capable of delivering an electrical field to any tissue adjacent to leads 20. In other examples, the electrodes of leads 20 may have different configurations. For example, the electrodes of leads 20 may have a complex electrode array geometry that is capable of producing shaped electrical fields. The complex electrode array geometry may include multiple electrodes (e.g., partial ring or segmented electrodes) around the perimeter of each lead 20, rather than a ring electrode. In this manner, electrical stimulation may be directed to a specific direction from leads 20 to enhance therapy efficacy and reduce possible adverse side effects from stimulating a large volume of tissue. In some examples, a housing of IMD 16 may include one or more stimulation and/or sensing electrodes. In alternative examples, leads 20 may have shapes other than elongated cylinders as shown in FIG. 1. For example, leads 20 may be paddle leads, spherical leads, bendable leads, or any other type of shape effective in treating patient 14.

IMD 16 may include a sensing module that senses bioelectrical signals within brain 12. The bioelectrical brain signals may reflect changes in electrical current produced by the sum of electrical potential differences across brain tissue. Examples of bioelectrical brain signals include, but are not limited to, an electroencephalogram (EEG) signal, electrocorticogram (ECoG) signal, a local field potential (LFP) sensed from within one or more regions of a patient's brain and/or action potentials from single cells within the patient's brain. In addition, in some cases, a bioelectrical brain signal can include a measured impedance of tissue of brain 12. In some examples, the bioelectrical brain signals may be used to determine whether patient 14 is in an anxiety state (also referred to herein as an anxiety episode) in which one or more symptoms of anxiety are present.

In some examples, leads 20 may include sensing electrodes positioned to detect the bioelectrical brain signal within one or more region of patient's brain 12. Alternatively, another set of implantable or external sensing electrodes may monitor the electrical signal. IMD 16 may deliver therapy and sense bioelectrical brain signals within the same or different target tissue sites of brain 12. For example, IMD 16 may detect an ECoG signal within the CG25 of brain 12 and deliver therapy to the VC/VS. The CG25 of brain 12 may also be referred to as the subgenual cingulate. As another example, IMD 16 may detect an EEG signal within the VC/VS of brain 12 and deliver therapy to the CG25. As another example, IMD 16 may to deliver therapy and sense within the VC/VS or the CG25 of brain 12.

As previously indicated, IMD 16 includes a signal generator that generates the electrical stimulation delivered to patient 14 via leads 20. In the example shown in FIG. 1, IMD 16 generates the electrical stimulation according to one or more therapy parameters, which may be arranged in a therapy program (or a parameter set). In particular, a signal generator (not shown) within IMD 16 produces the stimulation in the manner defined by the therapy program or group of programs selected by the clinician and/or patient 14. The signal generator may be configured to produce electrical pulses to treat patient 14. In other examples, the signal generator of IMD 16 may be configured to generate a continuous wave signal, e.g., a sine wave or triangle wave. In either case, IMD 16 generates the electrical stimulation therapy for DBS according to therapy parameter values defined by a particular therapy program.

A therapy program defines respective values for a number of parameters that define the stimulation. For example, the therapy parameters may include voltage or current pulse amplitudes, pulse widths, pulse rates, pulse frequencies, electrode combinations, and the like. IMD 16 may store a plurality of programs. In some cases, the one or more stimulation programs are organized into groups, and IMD 16 may deliver stimulation to patient 14 according to a program group. During a trial stage in which IMD 16 is evaluated to determine whether IMD 16 provides efficacious therapy to patient 14, the stored programs may be tested and evaluated for efficacy.

IMD 16 may include a memory to store one or more therapy programs (e.g., arranged in groups), and instructions defining the extent to which patient 14 may adjust therapy parameters, switch between programs, or undertake other therapy adjustments. Patient 14 may generate additional programs for use by IMD 16 via programmer 28 at any time during therapy or as designated by the clinician.

Generally, outer housing 34 of IMD 16 is constructed of a biocompatible material that resists corrosion and degradation from bodily fluids. IMD 16 may be implanted within a subcutaneous pocket close to the stimulation site. Although IMD 16 is implanted within a implanted within a subcutaneous pocket (e.g., above the clavicle) of patient 14 in the example shown in FIG. 1, in other examples, IMD 16 may be implanted within cranium. In addition, while IMD 16 is shown as implanted within patient 14 in FIG. 1, in other examples, IMD 16 may be located external to the patient. For example, IMD 16 may be a trial stimulator electrically coupled to leads 20 via a percutaneous lead during a trial period. If the trial stimulator indicates therapy system 10 provides effective treatment to patient 14, the clinician may implant a chronic stimulator within patient 14 for long term treatment.

Motion sensor 36 generates a signal indicative of patient activity (e.g., patient movement, activity level, or patient posture transitions). For example, motion sensor 36 may include one or more accelerometers (e.g., single axis, two-axis or three-axis accelerometers) capable of detecting static orientation or motion vectors in three-dimensions. An example accelerometer is a micro-electromechanical accelerometer. In other examples, motion sensor 36 may alternatively or additionally include one or more gyroscopes, pressure transducers, piezoelectric crystals, or other sensors that generate a signal that changes as a function of patient activity, e.g., physical activity or other motor activity.

IMD 16 delivers therapy to patient 14 to minimize the severity, duration or frequency of anxiety events resulting from an anxiety disorder, or even prevent the occurrence of the anxiety events. Depending upon the anxiety disorder with which patient 14 is afflicted, an anxiety event of patient 14 can be characterized by the presence of one or more specific patient activities during an anxiety episode. An anxiety episode may be a finite period of time during which the anxiety level of patient 14 is relatively high level (e.g., relative to a baseline state). In general, the known patient activities that occur during an anxiety event may be referred to as an activity component of an anxiety episode or anxiety event or a predetermined patient activity associated with the anxiety event. Engaging in the specific patient activity during an anxiety episode may help patient 14 decrease an anxiety level.

The patient activity that indicates an anxiety event can be a specific motor activity or a specific voice activity. The activity component of the anxiety episode is typically patient specific behavior. For example, OCD may be characterized by the presence of an obsession, such as intrusive thoughts, which may lead to compulsive behavior. Thus, OCD may be characterized by an overt action, such as a repetitive motion by patient 14, during an anxiety episode. Non-overt actions by patient 14 are also detectable as a specific patient activity during an anxiety episode. If patient 14 engages in a compulsive act, such as praying or counting, a non-overt action associated with the compulsive act can be detected. For example, a particular posture state may be detected during an anxiety episode, thereby indicting patient 14 is engaging in the specific patient act (e.g., patient 14 may assume a particular posture while praying). As another example, patient 14 may count with a finger motion, and the finger motion may be detected during an anxiety episode, thereby indicating patient 14 is engaging in the compulsive patient activity. In addition, specific voice activity resulting from a compulsion (e.g., praying, counting or repeating words) can also be detected during an anxiety episode.

Other types of patient activity may be characteristic of an anxiety event that is attributable to the anxiety disorder of patient 14. Some anxiety disorders are characterized by the presence of motor tics during an anxiety episode, where the motor tic can include involuntary, non-rhythmic, stereotyped movements. Thus, the specific patient activity can be a motor tic. As another example, PTSD may be characterized by the presence of a specific activity (e.g., as indicated by a pattern of motion or a specific voice activity) or a sudden increase in activity during a sleep state of patient 14. Another type of anxiety disorder is a panic disorder. A panic attack may be considered to be an anxiety event. While the physical manifestations of a panic attack may differ between patients, some patients may generally undertake a particular motor activity during a panic attack, such as assuming a particular posture or pacing.

Anxiety events are different than a general anxiety episode of patient 14 that is unrelated to the psychiatric disorder. For example, patient 14 may experience a general anxiety episode when patient 14 is worried or concerned about something, but the anxiety may not result in patient 14 engaging in a specific behavioral activity, such as a compulsion (e.g., a symptom of OCD) or a tic (e.g., a symptom associated with an anxiety disorder). A compulsion may be an overt act engaged in by patient 14 in a repetitive manner. The compulsion may be a motor-based behavior or can also be presented as mental acts (e.g., praying, counting, or repeating words). In some cases, patient 14 may engage in the compulsion until the anxiety diminishes, thereby indicating the end of the anxiety episode and anxiety event. Patients with OCD feel compelled to engage in the compulsion in order to minimize the anxiety that results from the intrusive thoughts.

A tic may be, for example, a motor (e.g., movement-based) tic, such as a repetitive and involuntary movement, which may have a sudden onset and/or may be a nonrhythmic movement affecting discrete muscle groups. A simple motor tic may include movements, such as brief jerking motion of the heart, arm, or leg (clonic tics), shoulder shrugging, hand clapping, or abdominal tensing. Motor tics, however, may also be more complex. For example, a complex motor tic may involve the use of multiple muscle groups, and can be characterized by a coordinated pattern of sequential muscle movement. A dystonic tic is an example of a complex motor tic. A dystonic tic can include abrupt bursts of twisting, pulling, and/or squeezing movements, which are sustained as unnatural postures of patient 14 for relatively short periods (e.g., are noncontinuous). A tic may also be a vocal tic that involves the involuntary utterance of one or more sounds, which may or may not be words.

Some patients experience a premonitory urge prior to the occurrence of a tic, where the urge may be brief or prolonged and characterized by an increased level of anxiety or tension. Execution of the tic by the patient may help provide a sensation of relief and reduction of the anxiety, e.g., similar to the mechanism by which a compulsion helps reduce the anxiety of a patient with OCD.

IMD 16 may detect an anxiety episode of patient 14 based on one or more characteristics of a bioelectrical brain signal or another physiological signal of patient 14. The physiological signal may indicate patient 14 is in a state in which patient 14 has a relatively high level of anxiety (e.g., relative to a baseline state). As described in further detail below, examples of physiological signals IMD 16 may sense to help IMD 16 detect an anxiety episode include, but are not limited to, bioelectrical brain signals, or signals indicative of a heart rate (e.g., as indicated by an electrocardiogram or a pulse oximeter), respiratory rate (e.g., as indicated by a transthoracic impedance sensor or a pulse oximeter), electrodermal activity (e.g., skin conductance level), changes in facial expression (e.g., as indicated by a facial electromyogram (EMG)), or facial flushing (e.g., as indicated by thermal sensing).

In some cases, a detected anxiety episode may not be an anxiety event for which therapy system 10 is implemented to mitigate or prevent. For example, an anxiety episode detected based on a bioelectrical brain signal may not be an OCD event, during which patient 14 engages in a compulsion. While many anxiety episodes may be detected based on the bioelectrical brain signal or other physiological parameter of patient 14, only some of those anxiety episodes may be considered an anxiety event of the anxiety disorder. For example, only some of the anxiety episodes may be perceived by patient 14 as being undesirable. As another example, a clinician may determine that only some of the anxiety episodes are caused by an anxiety disorder or severe enough to merit therapy delivery to help mitigate or prevent the occurrence of the anxiety event.

Anxiety can be commonly experienced by patient 14 throughout the ordinary course of daily living, and anxiety can be triggered by circumstances that are independent of the anxiety disorder of patient 14. In order to better monitor the anxiety disorder of patient 14, it is desirable to determine which of a plurality of detected anxiety episodes are anxiety events that are caused by the anxiety disorder. An anxiety episode detected based on bioelectrical brain signals or other physiological signals of patient 14 alone may merely indicate the presence of a relatively high level of anxiety and may not indicate the presence of a specific patient activity (e.g., compulsion or other behavior change) that is characterized by a physical movement (e.g., motor activity) or voice activity. Thus, monitoring only the bioelectrical brain signal or other physiological parameter of patient 14 may have low specificity in terms of detecting anxiety events for which therapy delivery to patient 14 is desirable.

As described herein, patient activity (e.g., voice activity or motor activity) may be monitored in order to help the clinician determine which of the many sensed anxiety episodes are anxiety events that are associated with a motor component. IMD 16, programmer 28 or another device may detect and identify a motor component during a detected anxiety episode of patient 14 based on a signal generated by motion sensor 36 (which may also be referred to as an activity sensor or a posture sensor). In this way, sensing activity of patient 14 via sensor 36 may help distinguish between anxiety episodes unrelated to an anxiety disorder and anxiety events of the anxiety disorder, which are characterized by the presence of a specific patient behavior. Motion sensor 36 helps to increase the specificity of anxiety event detection by IMD 16 by indicating which of a plurality of sensed anxiety episodes are accompanied by the patient specific motor behavior characteristic of the anxiety disorder.

Determining which anxiety episodes are anxiety events attributable to the anxiety disorder may help a clinician evaluate the anxiety disorder of patient 14. Temporally correlating the bioelectrical brain signal and the signal from motion sensor 36 that is indicative of patient activity may help the clinician distinguish between benign anxiety episodes that do not meet the anxiety event threshold (e.g., as indicated by the absence of a motor component during the episode) and undesirable anxiety events. Based on this information, the clinician alone or with the aid of programmer 28 may generate anxiety metrics that are useful for evaluating the anxiety disorder of patient 14 and, in some cases, the efficacy of therapy delivery by IMD 16. In some examples, IMD 16 automatically detects an anxiety event of patient 14 by detecting a motor component during an anxiety episode. In addition to or instead of merely monitoring the anxiety disorder of patient 14, the detection of the anxiety event may be used to control therapy delivery to patient 14.

An example of an anxiety metric is an indication of whether a detected anxiety episode was associated with an activity component (e.g., a motor component) that is characteristic of the anxiety disorder. An anxiety episode that was associated with an activity component may also be referred to as an anxiety event. As previously indicated, anxiety event is an anxiety episode for which therapy delivery by IMD 16 is desirable to help mitigate (e.g., minimize the severity or duration) or prevent. Another example of an anxiety metric is a percentage of a predetermined duration of time in which patient 14 was in an anxiety episode that is not matched with an activity component and the percentage of the predetermined duration of time in which patient 14 was in an anxiety event (e.g., an anxiety episode that is matched with a specific motor component). In addition, another example of an anxiety metric is the latency (e.g., a duration of time) between the onset of the anxiety episode and the onset of the specific patient activity that is characteristic of an anxiety event.

In the example shown in FIG. 1, motion sensor 36 is located within or on outer housing 34 of IMD 16. In other examples, motion sensor 36 may be implanted at any suitable location within patient 14 or may be carried externally to patient 14. The location for motion sensor 36 may be selected based on various factors, such as the type of motor component of an anxiety event that motion sensor 36 is implemented to detect. For example, if patient 14 has OCD that is characterized by the repeated washing of hands of patient 14 during an anxiety state, motion sensor 36 may be positioned proximate to the arms and/or hands of patient 14 to detect the repetitive hand washing motion. As another example, if patient 14 has a panic disorder that is characterized by pacing or walking in a particular pattern, motion sensor 36 may be positioned to detect motion of one or more limbs or torso motion involved during the pacing or other anxiety motor component of patient 14.

In general, motion sensor 36 is positioned to detect a motor activity associated with an anxiety event, such as the observable behaviors or complex rituals that are repeated multiple times and are readily associated with patient motion. In some examples, motion sensor 36 is positioned to detect a non-overt motor activity, such as an incidental patient motion that is associated with a compulsive thought. For example, during an anxiety event, patient 14 may suffer from compulsive thoughts, such as praying, counting or repeating words, which may be indirectly detected based on signals generated by motion sensor 36. As an example, if patient 14 taps fingers while counting, motion sensor 36 can pick up the finger tapping. Or if patient 14 occupies a particular posture while praying, the posture state may be detected based on the signal generated by motion sensor 36.

Motion sensor 36 may be separate from IMD 16 in some examples. A motion sensor that is physically separate from IMD 16 or leads 20 may communicate with IMD 16 via wireless communication techniques or a wired connection. In some examples, therapy system 10 includes more than one motion sensor 36. For example, multiple implanted or external motion sensors may be positioned to detect movement of multiple limbs (e.g., arms or legs) of patient 14.

In some examples, sensor 38, in addition to or instead of activity sensor 36, can be used to detect an activity component of an anxiety event of patient 14. Sensor 38 may be any suitable sensor that senses a physiological parameter associated with an anxiety event of patient 14. For example, sensor 38 can include a voice detector that detects voice activity of patient 14. In some cases, the anxiety disorder of patient 14 may not result in a specific motor activity during an anxiety event, but may instead result in a specific voice activity. In other examples, the anxiety disorder of patient 14 may result in both a motor activity and a voice activity during an anxiety event. Thus, in some examples, IMD 16 or programmer 28 receives a signal from sensor 38 instead of or in addition to motion sensor 36 to determine whether a detected anxiety episode is an anxiety event.

Sensor 38 may include any suitable voice activity sensor, such as a microphone, accelerometer tuned to detect movement of patient 14 indicative of vocal activity of patient 14, a vibration detector, or the like. The voice detector may, for example, detect a pattern of inflections in the patient's voice to determine whether patient 14 is engaging in compulsive behavior including a speech component or engaged in an involuntary vocal tic that is characteristic of an anxiety event. If patient 14 has an anxiety disorder, patient 14 may also have a co-morbid tic disorder, such as Tourette's syndrome, which can be characterized by the presence of motor tics and at least one vocal tic. In examples in which the anxiety disorder of patient 14 causes patient 14 to engage in a compulsive act that includes a speech parameter, such as repeating words, counting, praying, or a vocal tic, IMD 16 may determine whether patient 14 is in an anxiety state by detecting a particular pattern of voice activity based on a signal generated by sensor 38.

In some examples, sensor 38 may be a microphone (e.g., a crystal microphone, condenser microphone, a ribbon microphone, or other type of microphone) that generates an electrical signal indicative of sound, or a vibration detector (e.g., an acoustic sensor) that generates a signal indicative of movement of patient 14 resulting from patient speech. The microphone, vibration detector, accelerometer or other voice activity sensor 38 may be tuned to a specific frequency bandwidth to detect voice activity of patient 14 and minimize false positive detections of voice activity that may result from detecting voice activity of a person other than patient 14 or mischaracterizing other sounds as voice activity. In addition, a clinician or patient 14 may train the voice activity sensor or a processor of IMD 16 to discern between voice activity of patient 14 and other noise. For example, patient 14 may provide input (e.g., by tapping IMD 16 or providing input via programmer 28) to indicate when patient 14 is speaking, such that voice sensor 38 knows what activity is indicative of voice activity of patient 14.

Although shown as being physically separate from IMD 16 in the example shown in FIG. 1, in other examples, sensor 38 may be on or within an outer housing of IMD 16. Sensor 38 may be implanted within patient 14 at any suitable location (e.g., a subcutaneous implant site) or may be external (e.g., not implanted within patient 14). For example, if sensor 38 is a voice activity sensor that includes one or more of a vibration sensor, microphone or an acoustic sensor, sensor 38 may be positioned proximate to a chest or neck of patient 14, e.g., near the vocal cords and larynx (or other vocal muscles), but still in a discrete location. As another example, a vibration sensor, microphone, and/or an acoustic sensor may be positioned near IMD 16 or within IMD 16. As another example, in examples in which sensor 38 includes a microphone, sensor 38 may be positioned within programmer 28 if programmer 28 is a patient programmer that is carried by patient 14.

In some examples, system 10 may not include sensor 38. For example, in some cases, motion sensor 36 may be a voice activity sensor. For example, a processor of IMD 16 may be configured to detect movement of muscles related to patient speech, such as the larynx, the vocal cords or other respiratory, phonatory, and/or articulatory musculature based on a signal generated by motion sensor 36. The motion sensor may also detect vibrations generated during patient speech. In order to help limit false positive detections of the patient speech, motion sensor 36 may be configured to operate in a frequency bandwidth that includes the frequencies of the mechanical vibrations or other movement of patient 14 resulting from voice activity. The motion sensor may be tuned to a particular frequency bandwidth, such as by using a bandpass, low pass or high pass filter. An example of a bandpass filter is about 200 Hz to about 6 kHz.

While patient input may also be used to determine which detected anxiety episodes were associated with an activity component, the information from motion sensor 36 and/or voice activity sensor 38 can provide more objective and reliable information than the patient input. For example, patient 14 may periodically forget to provide input to indicate that an anxiety event occurred or may provide inaccurate input.

In some examples, patient input provided via programmer 28 may also be correlated with bioelectrical brain signal information in order to identify anxiety events. The patient input may indicate that an anxiety event occurred. For example, after the onset of a voice or motor activity that is characteristic of an anxiety event (e.g., a compulsion or tic), patient 14 may provide input via programmer 28 or IMD 16 (e.g., by tapping IMD 16 in a predetermined pattern, and IMD 16 may include a motion detector to detect the patient input) to indicate the anxiety event occurred. The input may also indicate a time at which the anxiety event occurred, such that the patient input may be temporally correlated with the bioelectrical brain signal information. One or more brain signal characteristics that are indicative of the anxiety events may be determined by temporally correlating the patient activity information, patient input, and bioelectrical brain signal information. The bioelectrical brain signal characteristics may be the signal characteristics temporally correlated with the patient activity information that is indicative of the anxiety event (e.g., an activity level exceeding a threshold level or substantially matching a template) and/or the patient input indicative of the onset of the anxiety event. The characteristic of the brain signal can include at least one of an amplitude threshold, a signal pattern, a power level within one or more frequency bands or a ratio of power levels in two or more frequency bands of the signal.

Example systems and techniques for receiving patient input to collect information related to the occurrence of a patient event, such as an anxiety event, are described in U.S. Patent Application Publication No. 2009/0082640 by Kovach et al., entitled, “PATIENT EVENT INFORMATION,” which was filed on Sep. 23, 2008 and is incorporated herein by reference in its entirety. As described in U.S. Patent Application Publication No. 2009/0082640 by Kovach et al., a processor of programmer 28 or another computing device may generate an event marker upon activation of an event indication button of programmer 28 by patient 14. For example, if patient 14 detects an intrusive thought or an impending compulsive act associated with an anxiety event, patient 14 may activate the event indication button, and, in response, the processor may generate an event marker. The patient may provide event information relating to the patient event. For example, the event information may include the type of anxiety event or motor component of the anxiety event, severity of anxiety event, duration of the anxiety event, drug type and dose taken prior to, during or after the occurrence of the anxiety event, a subjective rating of the efficacy of therapy that is delivered to manage the patient's anxiety disorder, and the like. Programmer 28 may provide a user interface that is configured to receive the event information from the patient, and, in some examples, may prompt the patient for the information.

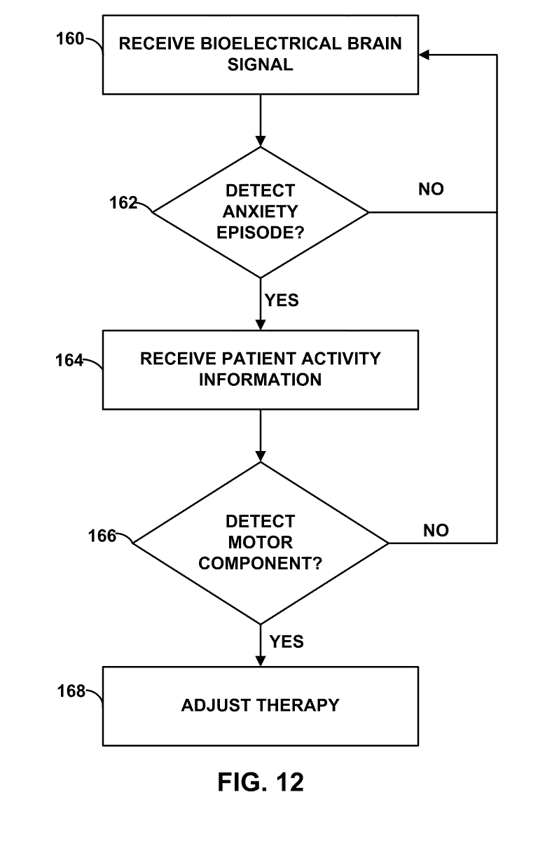

In addition to evaluating an anxiety disorder of a patient based on the detection of anxiety episodes and anxiety events, the techniques described herein may be used to generate an anxiety event detection algorithm with which IMD 16 automatically detects an occurrence of an anxiety event or a prospective anxiety event. For example, as described with reference to FIG. 12, the techniques described herein may be useful for determining one or more bioelectrical brain signal characteristics that are indicative of a target anxiety event that is associated with a motor component. Upon detecting the occurrence of the anxiety event or prospective anxiety event, IMD 16 may deliver therapy to patient 14 to help mitigate or prevent the anxiety event.

External programmer 28 wirelessly communicates with IMD 16 as needed to provide or retrieve therapy information. Programmer 28 is an external computing device that the user, e.g., the clinician and/or patient 14, may use to communicate with IMD 16. For example, programmer 28 may be a clinician programmer that the clinician uses to communicate with IMD 16 and program one or more therapy programs for IMD 16. Alternatively, programmer 28 may be a patient programmer that allows patient 14 to select programs and/or view and modify therapy parameters. The clinician programmer may include more programming features than the patient programmer. In other words, more complex or sensitive tasks may only be allowed by the clinician programmer to prevent an untrained patient from making undesired changes to IMD 16.

Programmer 28 may be a handheld computing device with a display viewable by the user and an interface for providing input to programmer 28 (i.e., a user input mechanism). For example, programmer 28 may include a small display screen (e.g., a liquid crystal display (LCD) or a light emitting diode (LED) display) that presents information to the user. In addition, programmer 28 may include a touch screen display, keypad, buttons, a peripheral pointing device or another input mechanism that allows the user to navigate though the user interface of programmer 28 and provide input. If programmer 28 includes buttons and a keypad, the buttons may be dedicated to performing a certain function, i.e., a power button, or the buttons and the keypad may be soft keys that change in function depending upon the section of the user interface currently viewed by the user. Alternatively, the screen (not shown) of programmer 28 may be a touch screen that allows the user to provide input directly to the user interface shown on the display. The user may use a stylus or their finger to provide input to the display.

In other examples, programmer 28 may be a larger workstation or a separate application within another multi-function device, rather than a dedicated computing device. For example, the multi-function device may be a notebook computer, tablet computer, workstation, cellular phone, personal digital assistant or another computing device that may run an application that enables the computing device to operate as a secure medical device programmer 28. A wireless adapter coupled to the computing device may enable secure communication between the computing device and IMD 16.

When programmer 28 is configured for use by the clinician, programmer 28 may be used to transmit initial programming information to IMD 16. This initial information may include hardware information, such as the type of leads 20, the arrangement of electrodes 24, 26 on leads 20, the number and location of motion sensor 36 within patient 14, the position of leads 20 within brain 12, the configuration of electrode array 24, 26, initial programs defining therapy parameter values, and any other information the clinician desires to program into IMD 16. Programmer 28 may also be capable of completing functional tests (e.g., measuring the impedance of electrodes 24, 26 of leads 20).

The clinician may also store therapy programs within IMD 16 with the aid of programmer 28. During a programming session, which may occur after implantation of IMD 16 or prior to implantation of IMD 16, the clinician may determine the therapy parameter values that provide efficacious therapy to patient 14 to address symptoms associated with the anxiety disorder. For example, the clinician may select one or more electrode combinations with which stimulation is delivered to brain 12. As another example, programmer 28 or another computing device may utilize a search algorithm that automatically selects therapy programs for trialing, i.e., testing on patient 14. During the programming session, patient 14 may provide feedback to the clinician as to the efficacy of the specific program being evaluated (e.g., trialed or tested) or the clinician may evaluate the efficacy based on one or more physiological parameters of patient 14 (e.g., heart rate, respiratory rate, or muscle activity). Programmer 28 may assist the clinician in the creation/identification of therapy programs by providing a methodical system for identifying potentially beneficial therapy parameter values.

Programmer 28 may also be configured for use by patient 14. When configured as a patient programmer, programmer 28 may have limited functionality (compared to a clinician programmer) in order to prevent patient 14 from altering critical functions of IMD 16 or applications that may be detrimental to patient 14. In this manner, programmer 28 may only allow patient 14 to adjust values for certain therapy parameters or set an available range of values for a particular therapy parameter.

Programmer 28 may also provide an indication to patient 14 when therapy is being delivered, when patient input has triggered a change in therapy or when the power source within programmer 28 or IMD 16 needs to be replaced or recharged. For example, programmer 28 may include an alert LED, may flash a message to patient 14 via a programmer display, generate an audible sound or somatosensory cue to confirm patient input was received, e.g., to indicate a patient state or to manually modify a therapy parameter.

Whether programmer 28 is configured for clinician or patient use, programmer 28 is configured to communicate to IMD 16 and, optionally, another computing device, via wireless communication. Programmer 28, for example, may communicate via wireless communication with IMD 16 using radio frequency (RF) telemetry techniques known in the art. Programmer 28 may also communicate with another programmer or computing device via a wired or wireless connection using any of a variety of local wireless communication techniques, such as RF communication according to the 802.11 or Bluetooth specification sets, infrared (IR) communication according to the IRDA specification set, or other standard or proprietary telemetry protocols. Programmer 28 may also communicate with other programming or computing devices via exchange of removable media, such as magnetic or optical disks, memory cards or memory sticks. Further, programmer 28 may communicate with IMD 16 and another programmer via remote telemetry techniques known in the art, communicating via a local area network (LAN), wide area network (WAN), public switched telephone network (PSTN), or cellular telephone network, for example.

Therapy system 10 may be implemented to provide chronic stimulation therapy to patient 14 over the course of several months or years. However, system 10 may also be employed on a trial basis to evaluate therapy before committing to full implantation. If implemented temporarily, some components of system 10 may not be implanted within patient 14. For example, patient 14 may be fitted with an external medical device, such as a trial stimulator, rather than IMD 16. The external medical device may be coupled to percutaneous leads or to implanted leads via a percutaneous extension. If the trial stimulator indicates DBS system 10 provides effective treatment to patient 14, the clinician may implant a chronic stimulator within patient 14 for relatively long-term treatment.

In addition to or instead of electrical stimulation therapy, IMD 16 may deliver a therapeutic agent to patient 14 to manage an anxiety disorder in addition to or instead of electrical stimulation therapy. In such examples, IMD 16 may include a fluid pump or another device that delivers a therapeutic agent in some metered or other desired flow dosage to the therapy site within patient 14 from a reservoir within IMD 16 via a catheter. The fluid pump may be external or implanted. The therapeutic agent may be used to provide therapy to patient 14 to manage a psychiatric disorder of patient 14, and may be delivered to the patient's brain 12, blood stream or tissue. As another example, a medical device may be an external patch that is worn on a skin surface of patient 14, where the patch elutes a therapeutic agent, which is then absorbed by the patient's skin. Other types of therapeutic agent delivery systems are contemplated. IMD 16 may deliver the therapeutic agent upon detecting an anxiety state that detects the anxiety state based on a bioelectrical brain signal or another patient parameter. The catheter used to deliver the therapeutic agent to patient 14 may include one or more electrodes for sensing bioelectrical brain signals of patient 14.

In the case of therapeutic agent delivery, the therapy parameters may include the dosage of the therapeutic agent (e.g., a bolus size or concentration), the rate of delivery of the therapeutic agent, the maximum acceptable dose in each bolus, a time interval at which a dose of the therapeutic agent may be delivered to a patient (lock-out interval), and so forth. Examples of therapeutic agents that IMD 16 may deliver to patient 14 to manage an anxiety disorder include, but are not limited to, selective serotonin reuptake inhibitor drugs, amitriptyline, amoxapine, benzodiazepines, bupropion, clomipramine, desipramine, doxepin, imipramine, monoamine oxidase inhibitors, maprotiline, mirtazapine, nefazodone, nortriptyline, protriptyline, trazodone, trimipramine, venlafaxines to manage OCD, anxiety disorders or MDD; alprazolam, buspirone, chlordiazepoxide, clonazepam, diazepam, halazepam, lorazepam, oxazepam, prazepam to manage anxiety disorders; and carbamazepine, depakote, divalproex sodium (valproic acid), gabapentin, lamotrigine, lithium carbonate, lithium citrate or topimarate to manage bipolar disorder. Other therapeutic agents may also provide effective therapy to manage the patient's anxiety disorder, e.g., by minimizing the severity, duration, and/or frequency of the patient's anxiety episodes. In other examples, IMD 16 may deliver a therapeutic agent to tissue sites within patient 14 other than brain 12.

While the remainder of the disclosure describes various systems, devices, and techniques for monitoring an anxiety disorder of patient 14 with respect to therapy system 10 of FIG. 1, the systems, devices, and techniques described herein are also applicable to other types of therapy systems, such as therapy systems that deliver a therapeutic agent to patient 14 to manage an anxiety disorder or therapy systems that only provide a notification to patient 14 upon detection of an anxiety state. In some cases, the therapy system may be used for monitoring bioelectrical brain signals and patient activity of patient 14 and may not include therapy delivery (e.g., stimulation delivery or therapeutic agent delivery) capabilities. The monitoring device may be useful for the clinician during, for example, initial evaluation of patient 14 to evaluate the anxiety disorder of patient 14.

In addition, while the remainder of the disclosure describes various systems, devices, and techniques that are directed to detecting an anxiety event by determining whether a specific motor activity is associated with a detected anxiety episode, in other examples, the systems, devices, and techniques described herein are also applicable to detecting an anxiety event by determining whether a specific voice activity is associated with a detected anxiety episode. Rather than detecting the specific patient activity via motion sensor 36 that is tuned to sense patient motion associated with a motor activity, the specific patient activity may be detected via voice activity sensor 38 or via motion sensor 36 that is tuned to sense patient motion associated with a voice activity.

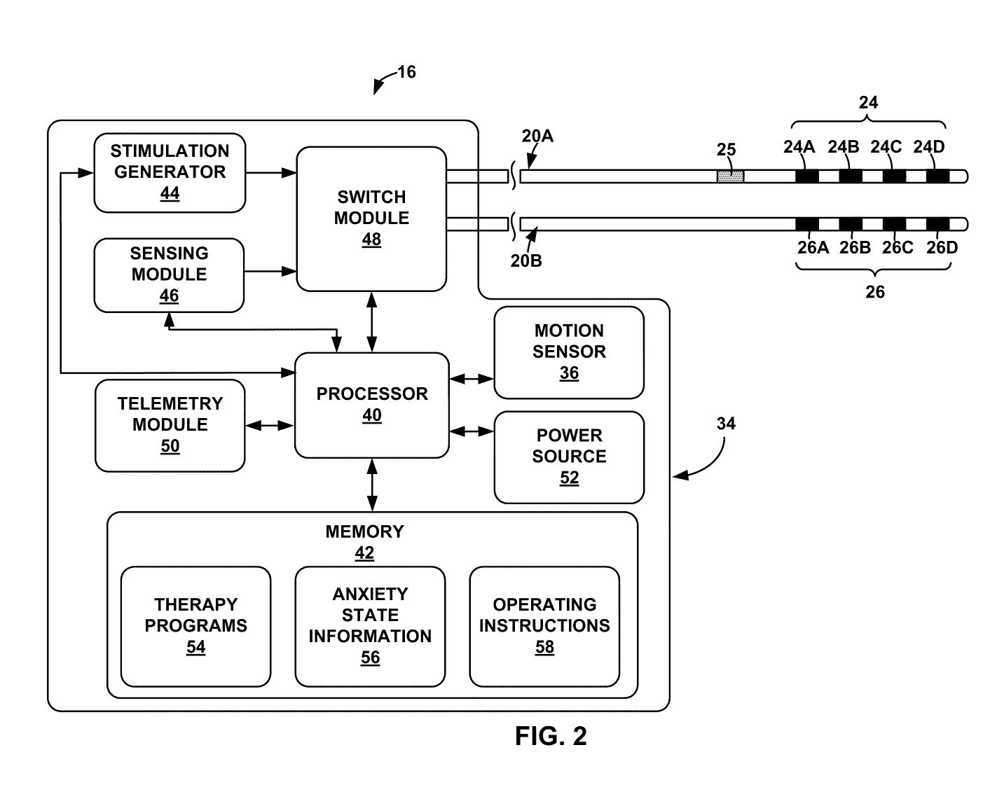

FIG. 2 is a functional block diagram illustrating components of an example IMD 16 in greater detail. In the example shown in FIG. 2, IMD 16 includes motion sensor 36, processor 40, memory 42, stimulation generator 44, sensing module 46, switch module 48, telemetry module 50, and power source 52. Memory 42 may include any volatile or non-volatile media, such as a random access memory (RAM), read only memory (ROM), non-volatile RAM (NVRAM), electrically erasable programmable ROM (EEPROM), flash memory, and the like. Memory 42 may store computer-readable instructions that, when executed by processor 40, cause IMD 16 to perform various functions described herein.

In the example shown in FIG. 2, memory 42 stores therapy programs 54, anxiety state information 56, and operating instructions 58 in separate memories within memory 42 or separate areas within memory 42. Each stored therapy program 54 defines a particular program of therapy in terms of respective values for electrical stimulation parameters, such as a stimulation electrode combination, electrode polarity, current or voltage amplitude, and, in if stimulation generator 44 generates and delivers stimulation pulses, the therapy programs may define values for a pulse width, pulse rate, and duty cycle of a stimulation signal. In some examples, the therapy programs may be stored as a therapy group, which defines a set of therapy programs with which stimulation may be generated. The stimulation signals defined by the therapy programs of the therapy group may be delivered together on an overlapping or non-overlapping (e.g., time-interleaved) basis.

Anxiety state information 56 stored by memory 42 includes information identifying each anxiety episode (also referred to as an anxiety state) detected by IMD 16 based on bioelectrical brain signals sensed by sensing module 46 and information identifying which of the detected anxiety episodes are anxiety events that are associated with a specific activity component. For example, the bioelectrical brain signals sensed by sensing module 46 during an anxiety episode and the electrical signals generated by motion sensor 36 that indicate patient motion or posture during an anxiety episode may be stored by memory 42 as anxiety state information 56. In addition, information relating to the actual occurrence of anxiety events, such as an indication generated by processor 40 when processor 40 detects specific motor activity (e.g., based on a signal from motion sensor 36 or patient input) during an anxiety state, may be stored by memory 42 as anxiety state information 56. In some examples, processor 40 may detect an anxiety state based on bioelectrical brain signals sensed by sensing module 46 via a subset of electrodes 24, 26. Thus, in some examples, processor 40 stores the bioelectrical brain signals as anxiety state information 56. Operating instructions 58 guide general operation of IMD 16 under control of processor 40, and may include instructions for measuring the impedance of electrodes 24, 26 and/or determining the distance between electrodes 24, 26.

IMD 16 is coupled to leads 20A and 20B, which include electrodes 24A-24D and 26A-26D, respectively (collectively “electrodes 24 and 26”). Although IMD 16 is coupled directly to leads 20, in other examples, IMD 16 may be coupled to leads 20 indirectly, e.g., via lead extension 18 (FIG. 1). In the example shown in FIG. 2, implantable medical leads 20 are substantially cylindrical, such that electrodes 24, 26 are positioned on a rounded outer surface of leads 20. As previously described, in other examples, leads 20 may be, at least in part, paddle-shaped (i.e., a “paddle” lead). In some examples, electrodes 24, 26 may be ring electrodes. If paddle leads are used, electrodes 24, 26 may be disc electrodes restricted to one side of the paddle, which may be more suitable for stimulating the surface of a cortex in some examples. In other examples, electrodes 24, 26 may be segmented or partial ring electrodes, each of which extends along an arc less than 360 degrees (e.g., 90-120 degrees) around the outer perimeter of the respective lead 20. The use of segmented or partial ring electrodes 24, 26 may also reduce the overall power delivered to electrodes 24, 26 by IMD 16 because of the ability to more efficiently deliver stimulation to a target stimulation site by eliminating or minimizing the delivery of stimulation to unwanted or unnecessary regions within patient 14.

The configuration, type, and number of electrodes 24, 26 illustrated in FIG. 2 are merely one example. For example, IMD 16 may be coupled to one lead with eight electrodes on the lead or three or more leads with the aid of bifurcated lead extensions. Electrodes 24, 26 are electrically coupled to stimulation generator 44 and sensing module 46 of IMD 16 via conductors within the respective leads 20A, 20B. Each of electrodes 24, 26 may be coupled to separate conductors so that electrodes 24, 26 may be individually selected, or in some examples, two or more electrodes 24 and/or two or more electrodes 26 may be coupled to a common conductor.

Processor 40 may include any one or more of a microprocessor, a controller, a digital signal processor (DSP), an application specific integrated circuit (ASIC), a field-programmable gate array (FPGA), discrete logic circuitry. The functions attributed to processors described herein may be embodied in a hardware device via software, firmware, hardware or any combination thereof. Processor 40 controls the stimulation generator 44 to generate and deliver electrical stimulation signals to patient 14 according to selected therapy parameters. Specifically, processor 40 controls stimulation generator 44 according to therapy programs 54 stored in memory 42 to apply particular stimulation parameter values specified by one or more programs, such as amplitude, pulse width, and pulse rate. In addition, processor 40 may also control stimulation generator 44 to deliver the electrical stimulation signals via selected subsets of electrodes 24, 26 with selected polarities. For example, switch module 48 may combine electrodes 24, 26 in various bipolar or multi-polar combinations to deliver stimulation energy to selected sites, such as sites within brain 12. In other examples, therapy programs are stored within programmer 28 or another computing device, which transmits the therapy programs to IMD 16 via telemetry module 50.

In some examples, stimulation generator 44 generates and delivers stimulation signals to an anterior limb of the internal capsule of brain 12 in order to manage symptoms of an anxiety disorder of patient 14, and stimulation therapy is delivered via a selected combination of the electrodes to the anterior limb of the internal capsule with electrical stimulation including a frequency of about 2 Hz to about 2000 Hz, a voltage amplitude of about 0.5 V to about 50 V, and a pulse width of about 60 μs to about 4 ms. Other stimulation targets within brain 28, other stimulation parameter values, and other therapy cycles are contemplated. Other ranges of therapy parameter values may also be useful, and may depend on the target stimulation site within patient 14, which may or may not be within brain 28. While stimulation pulses are described, stimulation signals may be of any form, such as continuous-time signals (e.g., sine waves) or the like.

In each of the examples described herein, if stimulation generator 44 shifts the delivery of stimulation energy between two therapy programs and/or two different electrode combinations, processor 40 of IMD 16 may provide instructions that cause stimulation generator 44 to time-interleave stimulation energy between the electrode combinations of the two therapy programs, as described in commonly-assigned U.S. Pat. No. 7,519,431 to Steven Goetz et al., entitled, “SHIFTING BETWEEN ELECTRODE COMBINATIONS IN ELECTRICAL STIMULATION DEVICE,” and filed on Apr. 10, 2006, the entire content of which is incorporated herein by reference. In the time-interleaved shifting example, the amplitudes of the stimulation signals delivered via the electrode combinations of the first and second therapy program are ramped downward and upward, respectively, in incremental steps until the amplitude of the second electrode combination reaches a target amplitude. The incremental steps may be different between ramping downward or ramping upward. The incremental steps in amplitude can be of a fixed size or may vary, e.g., according to an exponential, logarithmic or other algorithmic change. When the second electrode combination reaches its target amplitude, or possibly before, the first electrode combination can be shut off. Other techniques for shifting the delivery of stimulation signals between two therapy programs and/or electrode combinations may be used in other examples.

Processor 40 may control switch module 48 to apply the stimulation signals generated by stimulation generator 44 to selected combinations of electrodes 24, 26. In particular, switch module 48 may couple stimulation signals to selected conductors within leads 20, which, in turn, deliver the stimulation signals across selected electrodes 24, 26. Switch module 48 may be a switch array, switch matrix, multiplexer, or any other type of switching module configured to selectively couple stimulation energy to selected electrodes 24, 26 and to selectively sense bioelectrical brain signals with selected electrodes 24, 26. Hence, stimulation generator 44 is coupled to electrodes 24, 26 via switch module 48 and conductors within leads 20. In some examples, however, IMD 16 does not include switch module 48.

Stimulation generator 44 may be a single channel or multi-channel stimulation generator. In particular, stimulation generator 44 may be capable of delivering, a single stimulation pulse, multiple stimulation pulses or continuous signal at a given time via a single electrode combination or multiple stimulation pulses at a given time via multiple electrode combinations. In some examples, however, stimulation generator 44 and switch module 48 may be configured to deliver multiple channels on a time-interleaved basis. For example, switch module 48 may serve to time divide the output of stimulation generator 44 across different electrode combinations at different times to deliver multiple programs or channels of stimulation energy to patient 14.

Sensing module 46 is configured to sense bioelectrical brain signals of patient 14 via a selected subset of electrodes 24, 26. Processor 40 may control switch module 48 to electrically connect sensing module 46 to selected combinations of electrodes 24, 26. In this way, sensing module 46 may selectively sense bioelectrical brain signals with different combinations of electrodes 24, 26. As previously described, in some examples, processor 40 may detect an anxiety state of patient 14 via the sensed bioelectrical brain signal. In other examples, processor 40 may detect an anxiety state of patient 14 based on other physiological parameters of patient 14 in addition to or instead of a bioelectrical brain signal indicative of brain activity.

In addition, in some examples, sensing module 46 is configured to monitor a physiological signal of patient 14 in addition or instead of bioelectrical brain signals. Processor 40 can detect an anxiety episode of patient 14 based on the one or more other physiological parameters in addition to or instead of the bioelectrical brain signals. In some examples, sensing module 46 may comprise an external portion that is not implanted within patient 14 or may be implanted within patient 14, as shown in FIG. 2. In addition, in some examples, sensing module 46 may include portions both implanted and external to patient 14. In some examples, sensing module 46 may be incorporated in a common housing with IMD 16, as shown in FIG. 2, may include electrodes on an outer housing of IMD 16 or may be coupled to IMD 16 via leads 20 or separate leads. A sensing module that is separate from IMD 16 may communicate with IMD 16 and/or programmer 28 via a wired connection or via wireless communication techniques.

Other examples of a physiological parameter that sensing module 46 may monitor include a heart rate or a respiration rate. For example, sensing module 46 can generate a signal indicative of an ECG of patient 14 with the aid selected implanted electrodes 24, 26 and/or an electrode on an outer housing of IMD 16 (i.e., a “housing electrode”), or a signal indicative of respiration rate by determining an intrathoracic impedance via selected implanted electrodes 24, 26 and/or a housing electrode. In an anxiety episode, the patient's heart rate and respiration rate may increase relative to a baseline rate associated with a non-anxious mood state of patient 14. Thus, processor 40 can detect an anxiety episode by comparing the heart rate and/or respiration rate to a threshold value, which may be stored by memory 42.

In some examples, sensing module 46 can include an external portion for determining respiration rate and/or heart rate. For example, sensing module 46 can include an external respiration belt (e.g., a plethysmograpy belt) that generates a signal that varies as a function of the thoracic or abdominal circumference of patient 14 that accompanies breathing by patient 14. As another example, sensing module 46 can include an external electrocardiogram (ECG) belt that incorporates a plurality of electrodes for sensing the electrical activity of the heart of patient 14. The ECG belt 110 can be worn by patient 14. The heart rate and, in some examples, ECG morphology of patient 14 may be monitored based on the signal provided by ECG belt 110.

In some examples, sensing module 46 is also configured to sense muscle activity (e.g., EMG), the temperature of the patient's facial skin (e.g., a thermal sensing electrode), or the moisture level of the patient's skin (e.g., via electrodermal activity) via one or more implanted or external electrodes. For example, sensing module 46 may include electrodes positioned on the patient's face in order to detect the electrical potential generated by the patient's facial muscle cells when the patient's face contracts. That is, in some examples, sensing module 46 may include one or more electrodes positioned to detect EMG signals, which may indicate changes to the patient's facial expressions. Certain EMG signals may be associated with particular facial expressions, e.g., during a learning process. In some examples, sensing module 46 may include one or more thermal sensing electrodes positioned on the patient's face in order to detect facial flushing, and/or one or more sensing electrodes to detect electrodermal activity, which may indicate changes in conductivity of the patient's skin (e.g., attributable to perspiration).

Processor 40 may detect an anxiety episode of patient 14 with any one or more of the physiological parameters described above. A change in the patient's respiratory rate, heart rate, and galvanic skin response may indicate changes in the patient's overall arousal level or anxiety level. In addition, a change in the patient's facial expression (e.g., monitored by EMG) or facial flushing (e.g., monitored by thermal sensing) may indicate a change during an anxiety episode.

In the example shown in FIG. 2, IMD 16 includes motion sensor 36, which is on or within a housing that also encloses processor 40, stimulation generator 44, and sensing module 46. As previously described, in other examples, motion sensor 36 is connected to a lead and/or implanted separately from IMD 16 within patient 14, or may be external to patient 14. Motion sensor 36 may comprise any suitable device that generates an electrical signal that is indicative of patient motion or patient posture. For example, motion sensor 36 may comprise a single axis, two-axis or three-axis accelerometer, a piezoelectric crystal, a gyroscope or a pressure transducer. Signals from motion sensor 36 are provided to processor 40, which may detect a specific motor component of a patient anxiety episode using any suitable technique, such as template matching or comparison to a motion sensor output stored in memory 42.

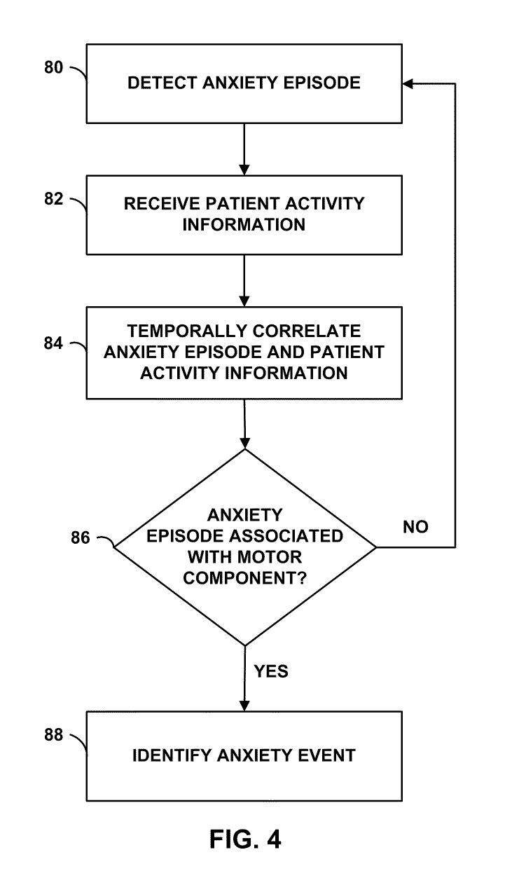

As described in further detail below, e.g., with reference to FIGS. 4-8, processor 40 may associate signals from motion sensor 36 with detected anxiety episodes. The motion sensor signals may be stored as anxiety state information 56 in memory 42 of IMD 16. In some examples, processor 40 of IMD 16 or a processor of another device, such as programmer 28, may determine various anxiety metrics based on the output from motion sensor 36 that is associated with an anxiety state. For example, processor 40 may determine an anxiety metric that indicates a percentage of time in which patient 14 was in an anxiety episode without the occurrence of an anxiety event and the percentage of time in which an anxiety event was occurring.

Telemetry module 50 supports wireless communication between IMD 16 and an external programmer 28 or another computing device under the control of processor 40. Processor 40 of IMD 16 may receive, as updates to programs, values for various stimulation parameters such as amplitude and electrode combination, from programmer 28 via telemetry module 50. The updates to the therapy programs may be stored within therapy programs 54 portion of memory 42. Telemetry module 50 in IMD 16, as well as telemetry modules in other devices and systems described herein, such as programmer 28, may accomplish communication by radiofrequency (RF) communication techniques. In addition, telemetry module 50 may communicate with external medical device programmer 28 via proximal inductive interaction of IMD 16 with programmer 28. Accordingly, telemetry module 50 may send information to external programmer 28 on a continuous basis, at periodic intervals, or upon request from IMD 16 or programmer 28. For example, processor 40 may transmit anxiety state information 56 to programmer 28 via telemetry module 50.

Power source 52 delivers operating power to various components of IMD 16. Power source 52 may include a small rechargeable or non-rechargeable battery and a power generation circuit to produce the operating power. Recharging may be accomplished through proximal inductive interaction between an external charger and an inductive charging coil within IMD 16. In some examples, power requirements may be small enough to allow IMD 16 to utilize patient motion and implement a kinetic energy-scavenging device to trickle charge a rechargeable battery. In other examples, traditional batteries may be used for a limited period of time.

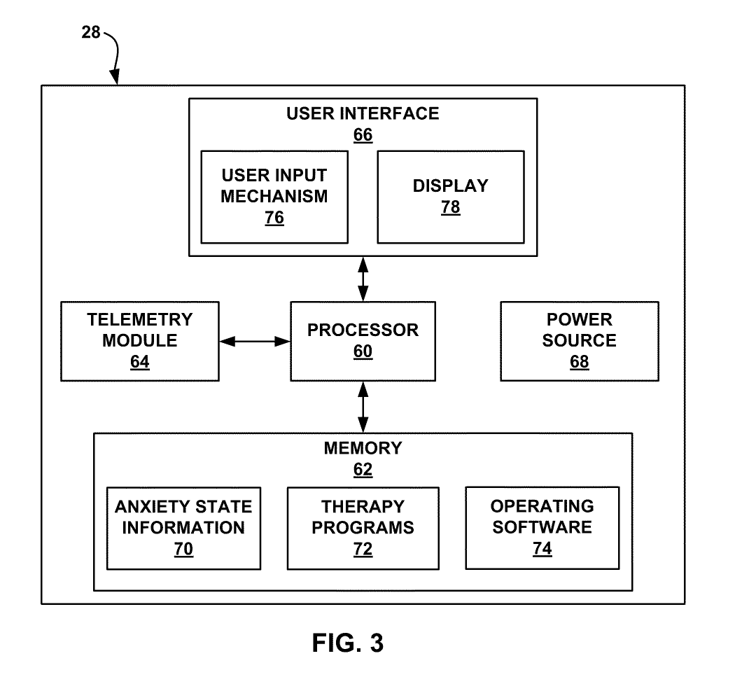

FIG. 3 is a conceptual block diagram of an example external medical device programmer 28, which includes processor 60, memory 62, telemetry module 64, user interface 66, and power source 68. Processor 60 controls user interface 66 and telemetry module 64, and stores and retrieves information and instructions to and from memory 62. Programmer 28 may be configured for use as a clinician programmer or a patient programmer. Processor 60 may comprise any combination of one or more processors including one or more microprocessors, DSPs, ASICs, FPGAs, or other equivalent integrated or discrete logic circuitry. Accordingly, processor 60 may include any suitable structure, whether in hardware, software, firmware, or any combination thereof, to perform the functions ascribed herein to processor 60.

A user, such as a clinician or patient 14, may interact with programmer 28 through user interface 66. User interface 66 includes user input mechanism 76 and display 78, such as a LCD or LED display or other type of screen, to present information related to the therapy, such as information related to bioelectrical signals sensed via a plurality of sense electrode combinations. Display 78 may also be used to present a visual alert to patient 14 that IMD 16 has detected an anxiety episode or anxiety event is about to occur. Other types of alerts are contemplated, such as audible alerts or somatosensory alerts. Input mechanism 76 is configured to receive input from the user. Input mechanism 76 may include, for example, buttons, a keypad (e.g., an alphanumeric keypad), a peripheral pointing device or another input mechanism that allows the user to navigate though user interfaces presented by processor 60 of programmer 28 and provide input.

Input mechanism 76 can include buttons and a keypad, where the buttons may be dedicated to performing a certain function, i.e., a power button, or the buttons and the keypad may be soft keys that change function depending upon the section of the user interface currently viewed by the user. Alternatively, display 78 of programmer 28 may be a touch screen that allows the user to provide input directly to the user interface shown on the display. The user may use a stylus or their finger to provide input to the display. In other examples, user interface 66 also includes audio circuitry for providing audible instructions or notifications to patient 14 and/or receiving voice commands from patient 14, which may be useful if patient 14 has limited motor functions. Patient 14, a clinician or another user may also interact with programmer 28 to manually select therapy programs, generate new therapy programs, modify therapy programs through individual or global adjustments, and transmit the new programs to IMD 16.

In some examples, at least some of the control of therapy delivery by IMD 16 may be implemented by processor 60 of programmer 28. For example, in some examples, processor 60 may receive patient activity information and bioelectrical brain signals from IMD 16 or from a sensing module that is separate from IMD 16. The separate sensing module may, but need not be, implanted within patient 14. In some examples, processor 60 may evaluate the patient activity information and bioelectrical brain signals from IMD 16 to determine which of a plurality of sensed anxiety states are anxiety episodes associated with the anxiety disorder of patient 14.

In addition, in some examples, a clinician, with the aid of programmer 28, may determine one or more brain signal characteristics indicative of anxiety episodes of patient 14 based on the patient activity information and bioelectrical brain signals generated by IMD 16. Programmer 28 or a clinician with the aid of programmer 28 may generate an anxiety episode detection algorithm based on the determined brain signal characteristics indicative of the anxiety episodes of patient 14. As previously discussed, the characteristic of the brain signal can comprise at least one of an amplitude threshold, a signal pattern, a power level within one or more frequency bands or a ratio of power levels in two or more frequency bands of the signal.

In the example shown in FIG. 3, memory 62 stores anxiety state information 70, therapy programs 72, and operating software 74. Operating software 74 may include instructions for operating user interface 66 and telemetry module 64, and for managing power source 68. Therapy programs 72 stored by memory 62 may include one or more therapy programs (or indications thereof) that are also stored by IMD 16 or additional therapy programs that may be programmed into IMD 16. Memory 62 may also store any therapy data retrieved from IMD 16 during the course of therapy, as well as anxiety state information 70 (e.g., anxiety episode indications received from patient 14 that indicate the time and date of an anxiety episode), sensed bioelectrical brain signals, and motion sensor information. The clinician may use this therapy data to determine the progression of the patient condition in order to plan future treatment for the anxiety disorder of patient 14. Memory 62 may include any volatile or nonvolatile memory, such as RAM, ROM, EEPROM or flash memory. Memory 62 may also include a removable memory portion that may be used to provide memory updates or increases in memory capacities. A removable memory may also allow sensitive patient data to be removed before programmer 28 is used by a different patient.

Wireless telemetry in programmer 28 may be accomplished by RF communication or proximal inductive interaction of external programmer 28 with IMD 16. This wireless communication is possible through the use of telemetry module 64. Accordingly, telemetry module 64 may be similar to the telemetry module contained within IMD 16. In alternative examples, programmer 28 may be capable of infrared communication or direct communication through a wired connection. In this manner, other external devices may be capable of communicating with programmer 28 without needing to establish a secure wireless connection.

Power source 68 delivers operating power to the components of programmer 28. Power source 68 may include a battery and a power generation circuit to produce the operating power. In some examples, the battery may be rechargeable to allow extended operation. Recharging may be accomplished by electrically coupling power source 68 to a cradle or plug that is connected to an alternating current (AC) outlet. In addition, recharging may be accomplished through proximal inductive interaction between an external charger and an inductive charging coil within programmer 28. In other examples, traditional batteries (e.g., nickel cadmium or lithium ion batteries) may be used. In addition, programmer 28 may be directly coupled to an alternating current outlet to receive operating power. Power source 68 may include circuitry to monitor power remaining within a battery. In this manner, user interface 66 may provide a current battery level indicator or low battery level indicator when the battery needs to be replaced or recharged. In some cases, power source 68 may be capable of estimating the remaining time of operation using the current battery.

FIG. 4 is a flow diagram of an example technique for monitoring an anxiety disorder of patient 14 by identifying anxiety events based on monitored patient activity. While processor 40 of IMD 16 is primarily referred to throughout the description of FIGS. 4, 10, and 12, in other examples, a processor of another device, such as processor 60 of programmer 28, may perform any part of the techniques described herein, alone or in combination with another device.

In accordance with the technique shown in FIG. 4, processor 40 detects an anxiety episode of patient 14 (80). In some examples, processor 40 detects an anxiety episode based on a bioelectrical brain signal sensed by sensing module 46 (FIG. 2) of IMD 16, where the bioelectrical brain signal indicates an anxiety level of patient 14 has increased. For example, processor 40 may compare the bioelectrical brain signal to a baseline state of the bioelectrical brain signal that indicates patient 14 is not in an anxiety episode.

Processor 40 may determine the baseline state of the bioelectrical brain signal (or other relevant physiological signal) during a time in which patient 14 is known to be in a non-anxious state, i.e., when an anxiety episode is not occurring. For example, processor 40 determine a bioelectrical brain signal characteristic that characterizes the baseline state of the signal when patient 14 provides input via programmer 28 that indicates patient 14 is not feeling anxious and that an anxiety episode is not occurring. Processor 40 may determine whether the bioelectrical brain signal returned to a baseline state using different techniques, which may depend on the physiological signal characteristic that characterizes the baseline state of the signal.

In examples in which the baseline state of the bioelectrical brain signal is characterized by an amplitude (e.g., a peak, instantaneous, mean, median or another amplitude), processor 40 may compare the amplitude value of the sensed bioelectrical brain signal at one time to the relevant amplitude value, which may be stored as a threshold value in memory 42 of IMD 16 or a memory of another device. Rather than continuously comparing the amplitude of the bioelectrical brain signal, processor 40 may periodically sample the bioelectrical brain signal and compare the relevant amplitude (e.g., the peak, instantaneous, mean, or median amplitude) of the bioelectrical brain signal for each sampled period to the threshold. The sample period may have any suitable duration, such as a few milliseconds or a few seconds. In some examples, if the amplitude value is greater than or equal to the stored threshold, processor 40 determines that an anxiety level of patient 14 has increased and that the anxiety episode is detected (80).

In examples in which the baseline state of the bioelectrical brain signal is characterized by a trend in the bioelectrical brain signal waveform, processor 40 may compare a trend in the bioelectrical brain signal to a template stored in memory 42. In one example, processor 40 implements a temporal correlation technique, during which processor 40 samples a sensed bioelectrical brain signal with a sliding window and compares the sample to a template stored in memory 42 to determine whether the sampled signal correlates well with the template. For example, processor 40 may perform a correlation analysis by moving a window along a digitized plot of the amplitude of the sensed bioelectrical brain signal at regular intervals, such as between about one millisecond to about one second intervals, to define a sample of the bioelectrical brain signal. The sample window may be slid along the plot of the bioelectrical brain signal waveform until a correlation is detected between the waveform of the baseline template and the waveform of the sample of the bioelectrical brain signal defined by the window. Upon detecting the correlation between the waveform of the baseline template and the waveform of the sample of the bioelectrical brain signal, processor 40 determines that an anxiety level of patient 14 has increased and that the anxiety episode is detected (80).

By moving the window at regular time intervals, multiple sample periods are defined. The correlation may be detected by, for example, matching multiple points between the template waveform and the waveform of the plot of the bioelectrical brain signal over time, or by applying any suitable mathematical correlation algorithm between the sample in the sampling window and a corresponding set of samples stored in the template waveform. As examples, if rate of change (i.e., the slope) of the monitored bioelectrical brain signal correlates to the slope of a trend template, the bioelectrical brain signal may indicate the presence of an anxiety episode of patient 14. As another example, if inflection points in the bioelectrical brain signal waveform substantially correlate to a template, the bioelectrical brain signal may indicate the presence of an anxiety episode of patient 14.

In examples in which the baseline state of the bioelectrical brain signal is characterized by a frequency band characteristic of the bioelectrical brain signal waveform, such as an energy level in a frequency band or a ratio of energy levels in more than one frequency band, processor 40 can compare the relevant frequency band characteristics of the bioelectrical brain signal waveform to a template or threshold value stored in memory 42 of IMD 16 or a memory of another device. Processor 40 may implement a frequency correlation technique, during which processor 40 analyzes the bioelectrical brain signal in the frequency domain and compares selected frequency components of the sensed bioelectrical brain signal to corresponding frequency components of the template signal. In each of the examples described above, the one or more templates or baseline amplitude values may be stored within memory 42 of IMD 16 or a memory of another device, such as programmer 28.

Each of the techniques for detecting an anxiety episode based on a characteristic of a bioelectrical brain signal are also applicable to detecting an anxiety episode based on a characteristic of another type of physiological signal, such as a signal indicative of a heart rate or a respiratory rate.

In some examples, processor 40 determines the time at which the anxiety episode began (the anxiety episode onset time) and the time at which the anxiety episode ended (the anxiety episode termination time) in order to determine a duration of the anxiety episode. Processor 40 can determine the anxiety episode onset time and the anxiety episode termination time using any suitable technique. In some examples, processor 40 determines the anxiety episode onset time to be the time at which processor 40 first detects the anxiety episode (80). While the anxiety episode may have begun prior to that time, processor 40 may only periodically determine whether the bioelectrical brain signal (or other physiological signal) is indicative of an anxiety episode. For example, processor 40 may determine whether the bioelectrical brain signal is indicative of an anxiety episode at a frequency of about 1 Hz or less, although other frequencies are contemplated. In addition, processor 40 can determine the anxiety episode termination time to be the time at which processor 40 first determines that the bioelectrical brain signal has returned to the baseline state, as indicated by the relevant threshold value or signal template.

In some examples, processor 40 groups two or more anxiety episodes together as a single anxiety episode. During an anxiety state in which an anxiety level of patient 14 is relatively high, the anxiety level (e.g., as indicated by a physiological signal amplitude value or pattern) may increase and decrease. As a result, the physiological signal may move between the baseline state and a state indicating the occurrence of an anxiety episode, and, therefore, the signal may indicate the occurrence of multiple anxiety episodes within a relatively short period of time. In order to detect more meaningful anxiety episodes, such as anxiety episodes having a duration sufficient to permit the specific motor activity of an anxiety event to occur, processor 40 can group two or more anxiety episodes occurring within a predetermined range of time of each other as a single anxiety episode.

For example, if a second anxiety episode occurs within a predetermined range of time after a first anxiety episode, such that the inter-episode interval is less than or equal to a stored threshold value (e.g., about one second to a minute or more), processor 40 groups the first and second anxiety episodes together. The threshold inter-episode interval for grouping anxiety episodes together can be stored in memory 42 of IMD 16 or another device. Any suitable number of anxiety episodes can be grouped together. Thus, if a third anxiety episode occurs within the inter-episode interval of the second anxiety episode, processor 40 can group the first, second, and third anxiety episodes together.

Processor 40 receives patient activity information (82), e.g., a signal generated by motion sensor 36 that changes as a function of patient activity (FIG. 2). Processor 40 can receive the signal generated by motion sensor 36 on a substantially continuous basis or on a periodic basis. After processor 40 detects the anxiety episode (80), processor 40 temporally correlates the anxiety episode and the patient activity information (84). Processor 40 determines whether the anxiety episode is associated with a motor component (86). As previously indicated, the motor component can be a compulsive act or a motor tic. Thus, processor 40 determines whether the patient activity information indicates that patient 14 engaged in a specific motor activity (e.g., a compulsion or a tic associated with Tourette's syndrome) during the anxiety episode. In other examples, processor 40 may determine whether the anxiety episode is associated with a specific vocal activity known to be indicative of an anxiety event.

Processor 40 can detect a motor component of an anxiety event based on the patient activity signal generated by motion sensor 36 (also referred to as a posture sensor or an activity sensor) using any suitable technique. As with the detection of an anxiety episode based on a bioelectrical brain signal, processor 40 can detect a motor component of an anxiety event using a characteristic of the signal generated by motion sensor 36. For example, processor 40 may compare an instantaneous, peak, mean or median amplitude value of the sensed patient activity signal to a threshold value that is in memory 42 of IMD 16 or a memory of another device. In some examples, if the relevant amplitude value is greater than or equal to the stored threshold, processor 40 determines that the motor component of the anxiety event is present.

As another example processor 40 may compare a trend in the patient activity signal to a template stored in memory 42. In one example, processor 40 implements a temporal correlation technique, during which processor 40 samples a patient activity signal with a sliding window and compares the sample to a template stored in memory 42 to determine whether the sampled signal correlates well with the template. Processor 40 may perform a correlation analysis by moving a window along a digitized plot of the amplitude of the patient activity signal at regular intervals, such as between about one millisecond to about one second intervals, to define a sample of the patient activity signal. The sample window may be slid along the plot of the patient activity signal waveform until a correlation is detected between the waveform of the baseline template and the waveform of the sample of the patient activity signal defined by the window. Upon detection the correlation between the waveform of the template and the waveform of the sample of the patient activity signal, processor 40 determines that the motor component of the anxiety event is present.

The correlation may be detected by, for example, matching multiple points between the template waveform and the waveform of the plot of the patient activity signal over time, or by applying any suitable mathematical correlation algorithm between the sample in the sampling window and a corresponding set of samples stored in the template waveform. As examples, if rate of change (i.e., the slope) of the monitored patient activity signal correlates to the slope of a trend template, the patient activity signal may indicate that patient 14 is engaged in a known, repetitive compulsive act, such as repetitive hand washing or repetitive flipping of a light switch. As another example, if inflection points in the patient activity signal waveform substantially correlate to a template, the patient activity signal may indicate the presence of an anxiety episode of patient 14.

In some examples, processor 40 detects the motor component (e.g., the predetermined patient activity) of an anxiety event based on one or more frequency domain components of the patient activity signal. For example, processor 40 can compare the energy level in a frequency band or a ratio of energy levels in more than one frequency band of the patient activity signal waveform to a template or threshold value stored in memory 42 of IMD 16 or a memory another device. Processor 40 may implement a frequency correlation technique, during which processor 40 analyzes the patient activity signal in the frequency domain and compares selected frequency components of the patient activity signal to corresponding frequency components of the template signal. In each of the examples described above, the one or more templates or baseline amplitude values may be stored within memory 42 of IMD 16 or a memory of another device, such as programmer 28.

Other types of patient activity signal characteristics indicative of an anxiety event are contemplated. For example, in some cases, the one or more activity signal characteristics indicative of an anxiety event are not predetermined and stored in IMD 16, but, rather, processor 40 determines whether the activity signal from sensor 36 indicates a sudden increase in patient activity level (e.g., as indicated by a slope exceeding a stored threshold), a sudden change in patient posture, or a repetitive activity during a detected anxiety episode. The sudden increase in patient activity level, change in patient posture or repetitive activity may indicate the occurrence of an anxiety event.

Processor 40 may implement any suitable statistical analysis to determine whether the patient activity signal is indicative of an anxiety event. In some examples, processor 40 determines whether the patient activity signal during an anxiety episode has a relatively low variance, e.g., by comparing a statistical metric (e.g., mean, median, lowest or highest amplitude value) for a current time period to the statistical metric (e.g., the mean, median, lowest or highest amplitude values) for previous time periods. If the statistical metric for the current time period is within a threshold range of the statistical metric for the previous time periods, processor 40 may determine that a detected anxiety episode is not an anxiety event. However, if the statistical metric for the current time period falls outside of the threshold range of the statistical metric for the previous time periods, processor 40 may determine that the patient activity signal is indicative of a sudden posture change or a sudden increase in activity, which may be indicative of an anxiety episode.

Processor 40 may also remove portions of the patient activity signal that correspond to a known activity of patient (e.g., walking to work), which are activities known to occur on a regular basis. For example, processor 40 may store the patient activity signal that corresponds to the known activity of patient 14 and compare the signal from activity sensor 36 to determine whether a detected increase in patient activity is attributable to a benign patient activity rather than an anxiety event.

In some examples, processor 40 determines the time at which the motor component began (the motor component onset time) and the time at which the motor component ended (the motor component termination time) in order to determine a duration of the compulsive act or other motor component of an anxiety event. Processor 40 can determine the motor component onset time and the motor component signal onset time using any suitable technique, such as the techniques described above for determining the anxiety episode onset time and the anxiety episode termination time.

In some examples, processor 40 groups two or more motor component detections together as a single motor component detection. During an anxiety event, patient 14 may engage in a noncontinuous motor activity, where each motor activity can be detected as a separate motor component. The noncontinuous motor activity may include, for example, a complex motor movement including sequential behaviors or a plurality of brief, transient tics in close succession. In addition, patient 14 may engage in more than one type of motor activity (e.g., a compulsion and/or tic) during an anxiety event. Thus, grouping multiple patient activities together that occur within a predetermined time range of each other as a single motor component may be useful. The patient activities that are grouped together may, but need not be, the same type of patient activity (e.g., the same type of compulsion).

For example, if a second motor component occurs within a predetermined range of time after a first motor component, such that the inter-component interval is less than or equal to a stored threshold value (e.g., about one second to a minute or more), processor 40 groups the first and second motor components together. The threshold inter-component interval for grouping motor components (or other activity components being monitored) together can be stored in memory 42 of IMD 16 or another device. Any suitable number of patient activities can be grouped together as a single activity component of an anxiety event. Thus, if a third motor component occurs within the inter-component interval of the second motor component, processor 40 can group the first, second, and third motor components together.

If the activity information correlated with the anxiety episode indicates that the anxiety episode is associated with a motor component, processor 40 identifies the detected anxiety episode as an anxiety event attributable to the anxiety disorder (88). In some examples, processor 40 generates and stores an anxiety event indication in memory 42 after identifying the anxiety event. The anxiety event indication may be, for example, a value, flag or signal that is stored in memory 42. If IMD 16 was delivering therapy to patient 14 when the anxiety event was detected, processor 40 can associate the anxiety event indication with the therapy program implemented by stimulation generator 44 to generate the stimulation signals at the time the anxiety event was detected.

If the activity information correlated with the anxiety episode does not indicate that the anxiety episode is associated with a motor component, processor 40 continues to monitor patient anxiety until another anxiety episode is detected (80). In some cases, anxiety episodes are separated by a certain amount of time to help define separate anxiety episodes in examples in which patient 14 is in a heightened anxiety state for a relatively long period of time.

A bioelectrical brain signal is referred to in the description of FIG. 4 for ease of description. In other examples, processor 40 can detect an anxiety episode of patient 14 based on a physiological parameter in addition to or instead of the bioelectrical brain signal. For example, as described above, processor 40 may detect an anxiety episode of patient 14 based on a change in the patient's respiratory rate, heart rate, and galvanic skin response may indicate changes in the patient's overall arousal level or anxiety level. In addition, a change in the patient's facial expression (e.g., monitored by EMG) or facial flushing (e.g., monitored by thermal sensing) may indicate an increase in an anxiety level associated with the anxiety episode.

Detecting an anxiety event of patient 14 may be useful for evaluating the progress of the patient's anxiety disorder. For example, programmer 28 or another computing device may generate trend information that indicates the number of anxiety event detections per some defined period of time (e.g., anxiety event detections per day) over time. Each anxiety event detection may be indicated by an anxiety event indication generated by processor 40 of IMD 16 or a processor of another device. The clinician may be able to quickly ascertain the progress of the patient's anxiety disorder by determining, for example, whether the number of anxiety event detections is decreasing over time, increasing over time, or remaining substantially the same. The determination of the trend of the anxiety event indications may drive a respective course of action, such as modifying therapy delivery.

In some examples, programmer 28 or another computing device can also generate a Venn diagram or another graphical display for presenting patient data that indicates the concordance between detected anxiety episodes and anxiety events. An additional graphical display that presents the concordance between patient input and anxiety events automatically detected based on sensor information may also be generated by programmer 28 or another device. The patient input may also be indicative of a premonitory urge that patient 14 experienced prior to exhibiting the motor tic component of an anxiety event. It may be useful for the clinician to determine the relative number of times that patient 14 experienced an obsession or premonitory urge prior to the occurrence of an anxiety event. The Venn diagram or other graphical display may present the relevant patient data in a meaningful format that enables the clinician to quickly review and ascertain relevant data, as well as the relationship between different data records (e.g., patient inputs and anxiety event detections).

As an example of a Venn diagram or other graphical display of patient data that may be generated, in patients with a tic disorder (e.g., Tourette's syndrome), the detected anxiety episodes may be reflective of a premonitory urge associated with the patient's motor tic. Programmer 28 may generate a Venn diagram that includes a first section associated with the detection of motor or vocal tics (e.g., by one or both sensors 36, 38) and a second section associated with the receipt of patient input indicating a premonitory urge. The overlap between the first and second sections may indicate the concordance between the detected motor or vocal tics and patient-perceived premonitory urges.

As another example, for a patient with OCD, the patient input may be indicative of an obsession that patient 14 experienced prior to engaging in the compulsive behavior. Programmer 28 may generate a Venn diagram that includes a first section associated with the detection of compulsions (e.g., by one or both sensors 36, 38) and a second section associated with the receipt of patient input indicating an occurrence of an obsession that patient 14 experienced prior to engaging in the compulsion. The overlap between the first and second sections may indicate the concordance between the detected compulsion and patient-perceived compulsions.

In some examples, the Venn diagram generated by programmer 28 and presented on display 78 (FIG. 3) may be interactive, such that a user may select a section of the Venn diagram to receive more information about the data associated with the section of the Venn diagram. The additional information may include, for example, patient rating of severity of a particular patient input, the date and time of the patient input or detected event, and the like. For example, if the Venn diagram includes a first section associated with the detection of motor or vocal tics and a second section associated with the receipt of patient input indicating a premonitory urge, a user may select the first section to receive more information about the detected tics, select the second section to receive more information about the patient input, or select the overlapping portions between the first and second sections to receive more information about the instances in which the detected tics and patient input overlapped.

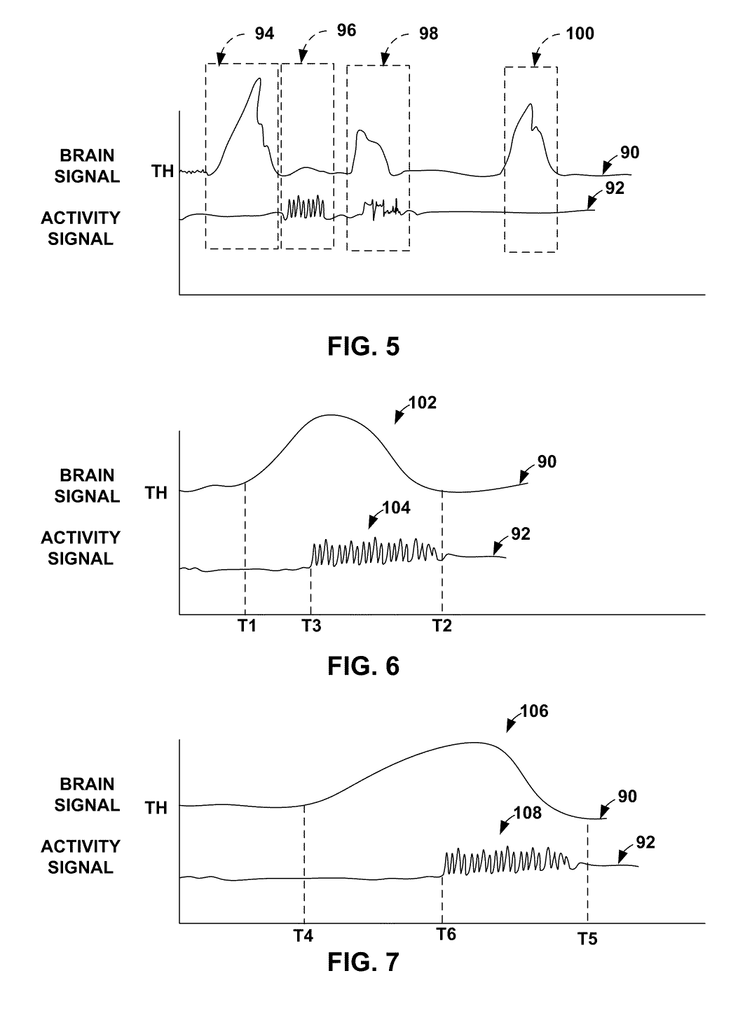

FIGS. 5-7 are conceptual illustrations of a temporally correlated bioelectrical brain signal 90, which can indicate the occurrence of an anxiety episode, and patient activity signal 92, which can indicate the occurrence of an anxiety event during the anxiety episode. FIG. 5 illustrates how a bioelectrical brain signal can indicate an anxiety episode that is not matched with a specific patient activity, and, therefore, is not an anxiety event related to the anxiety disorder of patient 14. For example, in first time period 94, brain signal 90 is indicative of a first anxiety episode (e.g., as indicated by a relatively high amplitude compared to amplitude threshold TH) during time period 94. However, during time period 94, activity signal 92 does not indicate the occurrence of a motor activity associated with the anxiety disorder of patient 14. Thus, processor 40 of IMD 16 may determine that the first anxiety episode is not an anxiety event because of the absence of the motor component.

In addition, FIG. 5 illustrates how patient activity may not be matched to an anxiety episode. For example, in second time period 96, patient activity signal 92 indicates an increased level of activity having a particular pattern. However, because the activity does not occur during an anxiety episode, processor 40 may either not detect the activity or may disregard the activity. In particular, processor 40 does not characterize the detected activity as a motor component of an anxiety event because the activity does not occur during the anxiety episode.

In third time period 98, bioelectrical brain signal 90 indicates a second anxiety episode (e.g., as indicated by a relatively high amplitude compared to amplitude threshold TH). The temporally correlated activity signal 92 indicates the occurrence of patient activity having a particular pattern. In the example shown in FIG. 5, activity signal 92 in third time period 98 is indicative of a specific motor activity of patient 14 that is associated with an anxiety event. Thus, when analyzing signals 90, 92, processor 40 of IMD 16 may determine that the second anxiety episode occurring during third time period 98 is associated with a motor component, and, therefore, an anxiety event occurred during third time period 98. If processor 40 controls stimulation generator 44 (FIG. 2) of IMD 16 based on detection of an anxiety event, as described in further detail with respect to FIG. 12, processor 40 may control stimulation generator 44 to generate and deliver stimulation to brain 12 of patient 14 upon determining that the second anxiety episode is an anxiety event. In other examples, processor 40 may merely generate and store an anxiety event indication.

In fourth time period 100, bioelectrical brain signal 90 is indicative of a third anxiety episode, but activity signal 92 does not indicate the occurrence of a motor activity associated with the anxiety disorder of patient 14. Thus, as with the first anxiety episode, processor 40 of IMD 16 may determine that the third anxiety episode is not an anxiety event because of the absence of an associated motor component.

As previously indicated, in some examples, processor 40 determines an anxiety episode duration, e.g., by determining the anxiety episode onset time and anxiety episode termination time. The duration of an anxiety episode stored for later analysis by a clinician. For example, a clinician, with the aid of programmer 28 or another computing device, can determine an anxiety metric that indicates a percentage of time during a sample time period (e.g., a single date, multiple days to weeks or months) that patient 14 was in an anxiety episode. The percentage of time that patient was in an anxiety episode may indicate the percentage of time in which patient 14 was in an anxious mood state with a relatively high anxiety level. The clinician may also determine an anxiety metric that indicates how many anxiety episodes that are not matched up with a motor component, i.e., how many anxiety episodes that are not anxiety events, were detected during the sample period of time. Another anxiety metric that may be determined based on a temporally correlated bioelectrical brain signal 90 and patient activity signal 92 includes the number of anxiety events detected within the sample period of time.

For anxiety events, additional anxiety metrics may be determined. For example, processor 40 of IMD 16, processor 60 of programmer 28 or a processor of another device may determine, for each anxiety event, the latency between the anxiety episode onset and the motor component onset. The latency indicates a duration of time in which patient 14 was in an anxiety episode before engaging in the specific motor activity that characterizes the anxiety disorder. As previously indicated, the motor activity can be a behavioral activity, such as a repetitive washing of hands or a repetitive movement of an object, or the motor activity can be involuntary, such as a tic. An voluntary vocal and/or motor tick may be a characteristic of Tourette's syndrome

FIG. 6 provides a conceptual illustration of the latency for an anxiety event in which a detected anxiety episode is associated with a motor component characteristic of the anxiety disorder of patient 14. In FIG. 6, bioelectrical brain signal 90 is indicative of an onset of anxiety episode 102 at time T1 and a termination of the anxiety episode at time T2. Processor 40 determines the duration of the anxiety episode as the duration of time between times T1 and T2. The duration of anxiety episode 102 can be an anxiety metric that is generated and stored for later analysis of the anxiety disorder of patient 14 by a clinician.

In the example shown in FIG. 6, patient activity signal 92 indicates an onset of motor component 104 at time T3. A duration of time between the onset of motor component 104 at time T3 and the termination of the anxiety episode at time T2 may be indicative of the severity of the anxiety event. For example, a first anxiety event exhibiting a shorter duration of time between times T3 and T2 than a second anxiety event may indicate that the first anxiety event was less severe because less time was spent in the compulsion or other motor component before the anxiety level was decreased. In some examples, processor 40 stores the duration of time between the onset of motor component 104 and the termination of the anxiety episode at time T2 as an anxiety metric. In some examples, the anxiety episode and activity component (e.g., a component) may not terminate at same time, although FIG. 6 illustrates an example in which they do. In those cases, the anxiety metric may be determined to be the time between time T3 and the detected end of the activity component.

A latency between the anxiety episode onset and the motor component onset is determined to be a duration of time between T1 and T3. The anxiety metric that indicate the latency between the anxiety episode onset and the motor component onset may indicate whether there was a gradual or rapid onset of a compulsion or other motor component. Thus, an anxiety metric can include the actual duration of time of the latency and/or a general classification of the anxiety event as including a gradual onset or a rapid onset of the motor component. In some examples, the clinician or another user may select the time ranges for classifying a particular motor component onset latency as a rapid onset and another as a gradual onset. In other examples, IMD 16 or programmer 28 are preprogrammed to include the values for the gradual and rapid onset classifications.

An anxiety event that includes a rapid onset of a motor component may require a different treatment than an anxiety event that includes a gradual onset of the motor component. The rapid motor component onset and gradual motor component onset may be indicative of different anxiety disorders or the relative severity of an anxiety disorder. For example, a rapid onset may be indicative of a panic attack or severe compulsive disorder, whereas the gradual onset may be indicative of a less severe compulsive disorder.

FIG. 7 is a conceptual illustration of an anxiety event including a more gradual motor component onset than the anxiety event shown in FIG. 6. In FIG. 7, bioelectrical brain signal 90 is indicative of an onset of anxiety episode 106 at time T4 and a termination of the anxiety episode at time T5. In the example shown in FIG. 7, patient activity signal 92 indicates an onset of motor component 108 of the anxiety event at time T6. A latency between the anxiety episode onset and the motor component onset is determined to be a duration of time between times T4 and times T6. In the examples shown in FIGS. 6 and 7, the duration of time between times T4 and times T6 is greater than the duration of time between times T3 and T1. Thus, in some examples, processor 40 may automatically classify the anxiety event shown in FIG. 7 as having a gradual motor component onset and the anxiety event in FIG. 6 as having a rapid motor component onset.

Instead of or addition to latency between the anxiety episode onset and the motor component onset, the difference in the intensity of the bioelectrical brain signal 90 at the onset of the motor component and during the presence of the motor component may be an anxiety metric for evaluating the anxiety disorder of patient 14. An increase in the intensity of bioelectrical brain signal 90 (e.g., an increase in amplitude or frequency of bioelectrical brain signal 90) may indicate the ability of patient 14 to resist performing compulsions. This anxiety metric assumes bioelectrical brain signal 90 is related to the level of anxiety of patient 14.

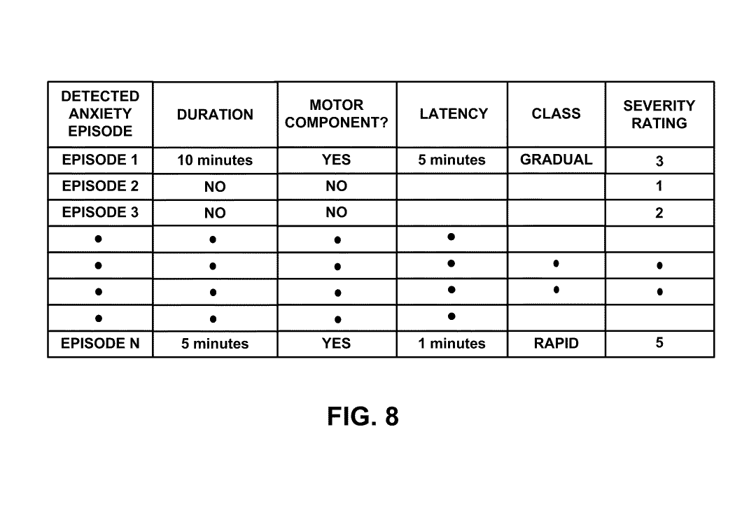

FIG. 8 is a conceptual illustration of a data structure that processor 60 of programmer 28 may generate and present to a user via 78 (FIG. 3) display of user interface 66 (FIG. 3). Although FIG. 8 illustrates a table, programmer 28 may also present anxiety state information via any suitable data structure. In addition, a computing device other than programmer 28 can generate the data structure shown in FIG. 8 and present the data to a user.

The table shown in FIG. 8 lists a plurality of detected anxiety episodes (“EPISODE 1,”EPISODE 2,” etc.), and anxiety metrics associated with each detected anxiety episode. In FIG. 8 “N” number of anxiety episodes are listed. In the example shown in FIG. 8, the anxiety metrics include a duration of each anxiety episode, and an indication of whether a motor component was detected during the anxiety episode, thereby indicating the anxiety episode was an anxiety event. In addition, for each anxiety event, the data structure shown in FIG. 8 provides an anxiety metric that indicates the latency between the onset of an anxiety episode and the onset of a motor component, as well as an anxiety metric that indicates whether the onset of the motor component was gradual or rapid. The anxiety metrics shown in FIG. 8 also include a severity rating for each detected anxiety episodes.

An anxiety metric is a value, classification or other parameter that can be used to evaluate an anxiety episode. The anxiety metrics help a clinician distinguish between a plurality of detected anxiety episodes and anxiety events, and determine which anxiety episodes or events were more severe than others. In some cases, the anxiety metrics provide objective parameters for evaluating anxiety episodes and comparing the anxiety episodes against each other. For example, in the example shown in FIG. 8, the duration of each anxiety episode and an indication of whether a motor component was detected during the anxiety episodes are objectively determined. The objective anxiety metrics help a clinician evaluate the patient's anxiety disorder independently of the patient's subjective input.

While patient 14 may still provide input for some or all of the detected anxiety episodes shown in FIG. 8, the anxiety metrics that are determined independently of the patient input provide more objective information for evaluating the anxiety episodes. Patient 14 may provide input relating to an anxiety episode with the aid of programmer 28 or a separate programmer. For example, as described in U.S. Patent Application Publication No. 2009/0082640 by Kovach et al, processor 40 of programmer 28 or another computing device may generate an event marker upon activation of an event indication button of programmer 28 by patient 14. For example, if patient 14 detects an intrusive thought or an impending compulsive act associated with an anxiety event, patient 14 may activate the event indication button, and, in response, the processor may generate an event marker. Processor 40 can generate an episode indication upon the activation of the event marker. Each episode indication can be a separate anxiety episode that is listed in a data structure similar to that shown in FIG. 8.

In some examples, patient 14 may not provide input indicating the occurrence of a specific patient activity component of an anxiety event until after the anxiety event has occurred (e.g., after a compulsion has ended). Thus, processor 60 of programmer 28 (or processor 40 of IMD 16 or another device) may associate the patient input indicating the occurrence of an anxiety event with a particular detected patient activity using any suitable technique. For example, processor 40 or 60 may automatically associate patient input with a patient activity signal sensed within a particular time range of the patient input. The time range may be selected to include relevant patient activity, given the compulsions or other activity components experienced by patient 14 during an anxiety event. For example, if patient 14 is known to engage in a compulsion that has a duration of about 10 minutes, the time range for associating patient input with a patient activity signal may be about 10 minutes or more.

Patient 14 can also provide information relating to the detected anxiety episode. For example, with the aid of programmer 28, patient 14 can provide an indication of type of anxiety episode (e.g., whether the anxiety episode was an anxiety event, such as a compulsion or a tic), a rating of the severity of anxiety episode, a duration of the anxiety episode, drug type and dose taken prior to, during or after the occurrence of the anxiety episode, a subjective rating of the efficacy of therapy that is delivered to manage the patient's anxiety disorder, and the like. With the aid of user interface 44 of programmer 28, patient 14 may assign an alphanumeric rating to an anxiety episode (e.g., on a scale of 1-5, where 5 indicates a severe anxiety episode and 1 indicates a relatively minor anxiety episode), as shown in FIG. 8, or assign a textual rating to an anxiety episode (e.g., via indicators, such as “MINOR,” “MODERATE,” “SEVERE,” and the like).

In some cases, patient 14 may not provide input relating to an anxiety episode detected by IMD 16. As indicated above, an anxiety level of patient 14 may increase, but may be unrelated to the anxiety disorder of patient 14. Thus, patient 14 may not associate the increased anxiety level as being undesirable in all examples. Instead, patient 14 may only provide input rating the severity of an anxiety episode in cases in which the anxiety episode is an anxiety event attributable to the anxiety disorder. For example, patient 14 may only provide input rating the severity of an anxiety episode in examples in which the episode is associated with an occurrence of a specific patient activity, such as a compulsion or a motor tic.

The “SEVERITY RATING” anxiety metric shown in FIG. 8 can be based on the subjective patient (or other user) input and/or based on objective anxiety metrics, such as the duration of the anxiety episode, latency, class, and the like. In some examples, the severity of an anxiety episode may be automatically detected based on one or more monitored physiological parameters values of patient 14, such as the amplitude of the patient activity signal generated by motion sensor 36 detected during an anxiety episode. Processor 40 of IMD 16 or processor 60 of programmer 28 may determine the severity of the anxiety event and automatically record the severity within the respective memory 42, 62. Severity may be categorized in terms of a graduated scale (e.g., a numerical scale) or another suitable scale. Alternatively, processor 40 or 60 may merely record the patient activity signal and clinician or another computing device may determine the severity of the patient's anxiety event, if any, at the time the event marker was generated.

In some examples, the severity of the anxiety episode may be automatically determined based on a plurality of anxiety metrics. For example, processor 40 or 60 may assign different weights to the different anxiety metrics to determine a severity metric.

Processor 60 may determine the duration of the anxiety episode using any suitable technique. In one example, as described above, either processor 40 of IMD 16 or processor 60 of programmer 28 determines the time at which the anxiety episode was first detected and the time at which the anxiety episode is no longer detected, and the duration between those times is recorded as the duration of anxiety episode. Similarly, processor 40 of IMD 16 or processor 60 of programmer 28 can determine the latency metric using any suitable technique, such as by determining the duration of time between the time at which the anxiety episode was first detected and time at which the motor component was first detected.

The classification of an anxiety event (e.g., an anxiety episode including a motor component) as having a gradual or rapid onset can be determined using any suitable technique. As described above with respect to FIGS. 6 and 7, processor 40 of IMD 16 or processor 60 of programmer 28 may compare the latency to a threshold value or a ranges of values. If the latency is greater than (and in some examples, equal to) the threshold value or falls within a first range of values, processor 40 or 60 may determine that the onset of the motor component was rapid. If the latency is less than (and in some examples, equal to) the threshold value or falls within a second range of values, processor 40 or 60 may determine that the onset of the motor component was gradual. The clinician may select ranges of values and/or the threshold values, for classifying a particular latency as a rapid onset and another as a gradual onset or IMD 16 or programmer 28 may be preprogrammed to include the values.

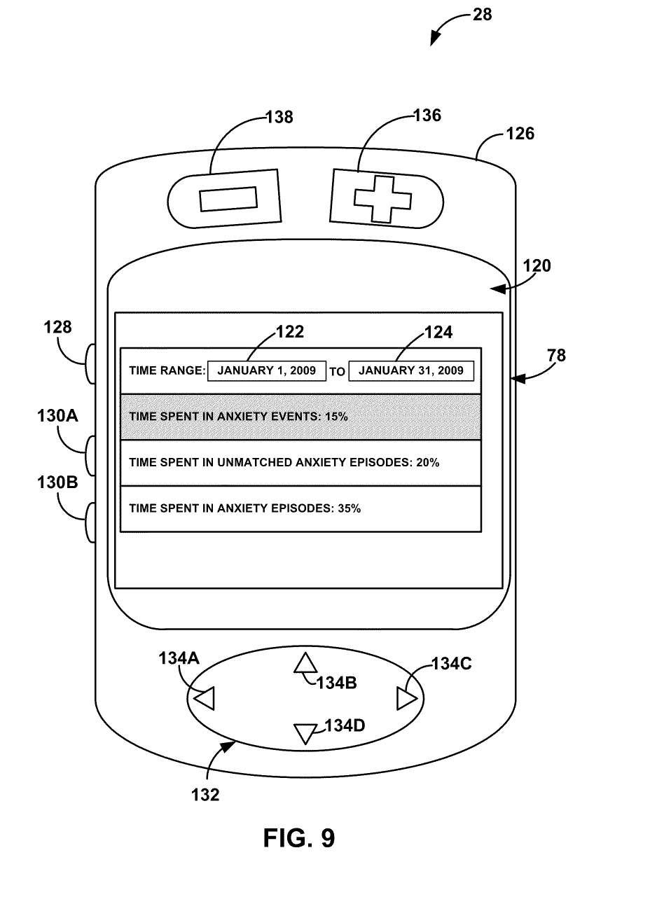

FIG. 9 is a schematic illustration of programmer 28, which illustrates a graphical user interface (GUI) 120 presented on display 78 of programmer 28. GUI 120 presents information relating to the anxiety episodes of patient 14 detected within a selected time range. In the example shown in FIG. 9, a user (e.g., a clinician or patient 14) can select the desired time range by selecting a start date 122 and an end date 124. Start date 122 and end date 124 may be text boxes in which the user can input the desired dates, or may be pull-down menus or calendar views from which the user can select the desired dates from predetermined options. For the selected time range, processor 60 generates and presents a display via GUI 120 that indicates the amount of time patient 14 spent in anxiety events, unmatched anxiety episodes, and anxiety episodes, which includes both anxiety events and unmatched anxiety episodes (e.g., anxiety episodes in which no motor component is detected such that the anxiety episodes are not considered anxiety events).

The example shown in FIG. 9 presents the time spent in each of the different categories of an anxiety state as a percentage of the selected time range. For example, in FIG. 9, GUI 120 indicates that for the time range from Jan. 1, 2009 to Jan. 31, 2009, patient 14 was in an anxiety episode about 35% of time, indicating patient 14 was highly anxious. In addition, GUI 120 indicates that of that 35% of the time spent in anxiety episodes, 20% of the time range was spent in an unmatched anxiety episode, which may be attributable to general anxiety and not an anxiety state that caused patient 14 to change behavior (e.g., as indicated by an anxiety event). GUI 120 also indicates that of that 35% of the time spent in anxiety episodes, 15% of the time range was spent in an anxiety event. During an anxiety event, patient 14 experiences a heightened level of anxiety in combination with a specific patient activity, such as a compulsive act, a motor tic or a vocal tic.

Processor 60 of programmer 28 may determine the time spent in anxiety events, unmatched anxiety episodes, and anxiety episodes using any suitable technique. In some examples, processor 60 uses the anxiety metrics shown in the data structure of FIG. 8 to determine the time spent in anxiety episodes, the time spent in unmatched anxiety episodes, and the time spent in anxiety events. For example, the duration of each anxiety episode detected during the selected time range may be added to determine total time spent in an anxiety episode. Processor 60 can determine the time spent in anxiety events by determining the sum of the durations of each anxiety episode matched with an activity component, e.g., each anxiety episode in which a motor component was detected. In addition, processor 60 can determine the time spent in an unmatched anxiety episode by determining the sum of the durations of each anxiety episode for which no motor component was detected.

The amount of time patient 14 spent in anxiety events, unmatched anxiety episodes, and anxiety episodes, which includes both anxiety events and unmatched anxiety episodes may be useful for evaluating the patient anxiety disorder, determining the progression of the anxiety disorder, and/or evaluating the efficacy of therapy delivery by IMD 16 in examples in which IMD 16 is implemented. In some cases, IMD 16 does not deliver therapy but merely monitors the patient anxiety disorder using the techniques described herein.

A clinician may determine the amount of time patient 14 spent in anxiety events, unmatched anxiety episodes, and anxiety episodes for different time ranges and, for example, determine whether the amount of time spent in anxiety events is increasing, thereby indicating a worsening of the patient's anxiety disorder or a decrease in efficacy of therapy delivery. In some examples, processor 60 of IMD 16 or another computing device can generate a graphical representation of the percentage of time spent in anxiety event, unmatched anxiety episodes, and/or anxiety episodes as a function of time. This type of display presented on display 78 of programmer 28 may help the clinician ascertain the progression of the anxiety disorder relatively quickly. Other types of information for evaluating the patient's anxiety disorder may also be derived from the determination of the amount of time patient 14 spent in anxiety events, unmatched anxiety episodes, and anxiety episodes.

In some examples, the patient condition may be evaluated based on the number of or frequency of occurrence of at least one of the anxiety events, unmatched anxiety episodes or anxiety episodes within a particular range of time (e.g., a day, a week, a month, and so forth). The clinician may select the range of time or programmer 28 may automatically select the range of time for determining the frequency of the at least one of the anxiety events, unmatched anxiety episodes or anxiety episodes.

As shown in FIG. 9, programmer 28 also includes housing 126, power button 128, contrast buttons 130A, 130B, control pad 132 with directional buttons 134A, 134B, 134C, and 134D, increase button 136, and decrease button 138. Housing 126 may substantially enclose the components of programmer 28, such as processor 60 and memory 62. A user may depress power button 128 to turn programmer 28 on or off. Programmer 28 may include safety features to prevent programmer 28 from shutting down during a telemetry session with IMD 16 or another device in order to prevent the loss of transmitted data or the stalling of normal operation. Alternatively, programmer 28 and IMD 16 may include instructions for handling possible unplanned telemetry interruption, such as battery failure or inadvertent device shutdown.

As previously indicated with respect to FIG. 3, display 78 may be a liquid crystal display (LCD), touch screen display, or another type of monochrome or color display capable of presenting information to a user, e.g., a clinician. Contrast buttons 130A, 130B may be used to control the contrast of display 78. In addition to displaying a list of trialed therapy programs and associated evaluation metrics, processor 60 of programmer 28 may also present information regarding the type of IMD 16, operational status of IMD 16, patient data, and operational status of clinician programmer 28 on display 78.

Control pad 132 allows the user to navigate through items presented on display 78. For example, the clinician may press control pad 132 on any of arrows 134A-134D in order to move between items presented on display 78 or move to another screen not currently shown by display 78. For example, the clinician may depress or otherwise activate arrows 134A, 134C to navigate between screens of GUI 120, and depress or otherwise activate arrows 134B, 134D to scroll through the therapy programs presented by GUI 120. The clinician may press the center portion of control pad 132 in order to select any highlighted element in GUI 120. For example, the clinician may scroll to and select “TIME SPENT IN ANXIETY EVENTS,” which is shown to be highlighted in FIG. 9, in order to retrieve more information about the anxiety events detected by IMD 16 during the selected time range, such as the severity of the anxiety events, the latency, and other anxiety metrics associated with the anxiety events. In other examples, scroll bars, a touch pad, scroll wheel, individual buttons, a stylus (in combination with a touch screen display 78) or a joystick may perform the complete or partial function of control pad 132.

Increase button 136 and decrease button 138 provide input mechanisms for a user, such as clinician or patient 14. In general, depressing decrease button 138 one or more times may decrease the value of a highlighted therapy parameter and depressing increase button 136 one or more times may increase the value of a highlighted therapy parameter that is presented on display 78. While buttons 136, 138 may be used to control the value of any therapy parameter, the user may also utilize buttons 136, 138 to select or generate particular programs for testing during a therapy programming session.

Programmer 28 may take other shapes or sizes not described herein. For example, programmer 28 may take the form of a clam-shell shape, similar to cellular phone designs. In any shape, programmer 28 may be capable of performing the requirements described herein. Furthermore, in other examples, the buttons of programmer 28 may perform different functions than the functions provided in FIG. 9 as an example. In addition, other examples of programmer 28 may include different button layouts or number of buttons. For example, display 78 may be a touch screen that incorporates all user interface and user input mechanism functionality.

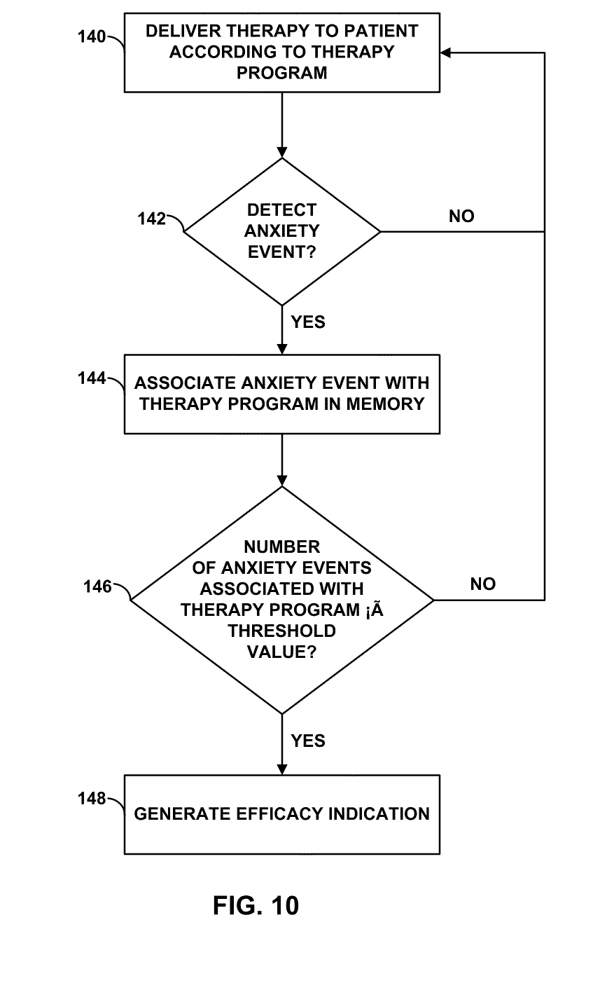

In some examples, processor 40 of IMD 16 or processor 60 of programmer 28 may determine the number of anxiety events detected during therapy delivery by IMD 16 and associate the anxiety events with a therapy program. FIG. 10 is a flow diagram of an example technique for associating anxiety events with therapy programs. While FIG. 10 is described with reference to processor 40 of IMD 16, in other examples, processor 60 of programmer 28 or another device may perform any part of the technique shown in FIG. 10.

Processor 40 controls stimulation generator 44 to deliver therapy to patient 14 according to a set of therapy parameter values, referred to herein as a therapy program (140). The therapy program may be selected from a plurality of therapy programs stored by memory 42 of IMD 16 or a memory of another device. In some examples, stimulation generator 44 delivers stimulation signals to patient 14 on a substantially continuous basis or on a periodic basis, e.g., according to a predetermined schedule. In other examples, processor 40 controls stimulation generator 44 to deliver therapy to patient 14 as needed, as described with reference to FIG. 12.

During the course of therapy delivery by IMD 16, processor 40 may detect an anxiety event (142), e.g., using the technique described with reference to FIG. 4. Upon detecting the anxiety event, processor 40 may associate an anxiety event indication with the currently implemented therapy program and store the association memory (144). Determining the number of anxiety events (as indicated by the number of anxiety event indications) that are detected during therapy delivery according to a specific therapy program may help a clinician evaluate the efficacy of the therapy programs or compare therapy programs. For example, a clinician may determine that a goal of therapy delivery by IMD 16 is to minimize the number of anxiety events that patient 14 experiences. Thus, if a particular therapy program is associated with a relatively high number of anxiety events (or even any anxiety events), the clinician may determine that the therapy program is not efficacious for patient 14.

In some examples, the clinician may compare the number of anxiety events to a predetermined threshold value to determine whether the therapy program is efficacious. For example, as shown in FIG. 10, processor 40 can determine whether the number of anxiety events associated with the therapy program is greater than or equal to a threshold value (146). In some examples, processor 40 compares the number of anxiety events associated with a particular therapy program for a selected range of time of therapy delivery to the threshold value. In other examples, processor 40 compares the number of anxiety events associated with a particular therapy program for the entire time in which IMD 16 implemented the therapy program to the threshold value.

If the number of anxiety events associated with the therapy program is greater than or equal to the threshold value, processor 40 generates an efficacy indication (148), which is associated with the therapy program. The efficacy indication may be, for example, a flag, value or signal indication that indicates that the therapy program may not be efficacious. The efficacy indications help identify the therapy programs that may need to be modified.

The technique shown in FIG. 10 is useful for comparing the efficacy of a plurality of therapy programs. For example, IMD 16 may associate anxiety events with a plurality of therapy programs using the technique shown in FIG. 10, and then, at a later time, a clinician may compare the efficacy of the therapy programs with each other based on the number of associated anxiety events. In some examples, the more anxiety events associated with a therapy program, the less efficacious the therapy program.

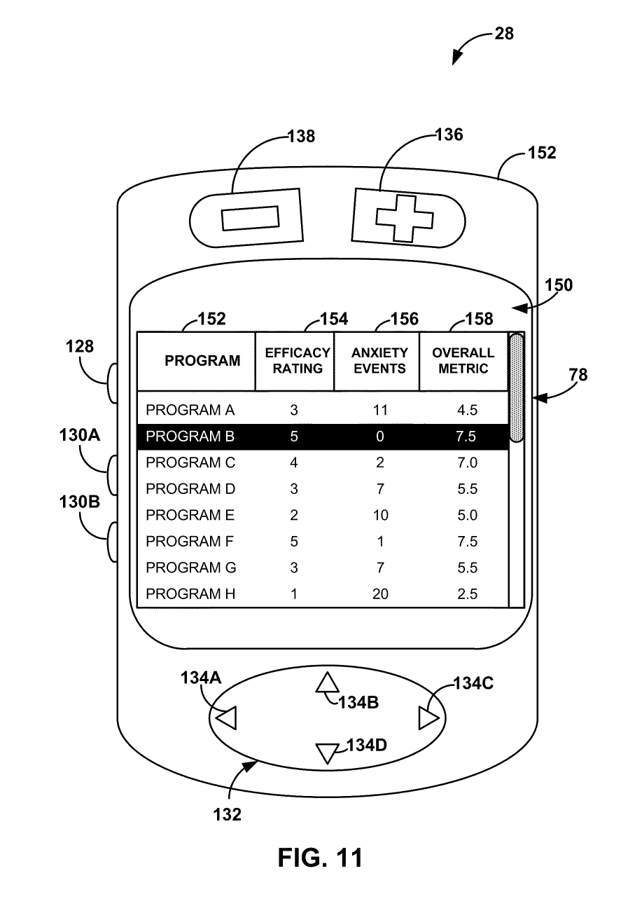

FIG. 11 is another schematic illustration of programmer 28, which illustrates GUI 140 presented on display 78, where GUI 140 provides a list of therapy programs implemented by IMD 16 and the number of anxiety events associated with the therapy program. A clinician may evaluate a plurality of therapy programs based on associated anxiety event information with the aid of GUI 150. GUI 150 includes a list of therapy programs 152 tested during an evaluation session, which may be any suitable duration of time, such as days to weeks or even months. It may be desirable to test therapy programs over the course of at least a few days in order to give patient 14 the opportunity to be exposed to various environmental triggers or cues that may cause anxiety events.

The therapy programs are designated PROGRAM A, PROGRAM B, and so forth in FIG. 11. GUI 150 also presents efficacy ratings 154 for the therapy programs, a number of anxiety events 156 associated with each therapy program, and an overall anxiety metric associated with the respective therapy program. The efficacy rating, number of anxiety events 156, and overall metric may be considered to be evaluation metrics of the respective therapy program. The clinician may evaluate the therapy programs 152 based on the evaluation metrics.

Patient 14, the clinician or another user may utilize control pad 132, buttons 136, 138 or display 78 in examples in which display 78 comprises a touch screen to input efficacy ratings for each therapy program or to input other evaluation metrics. The input relating to the evaluation metric may take place during therapy delivery according to the respective therapy program or after therapy delivery according to the respective therapy program. In the example shown in FIG. 11, the efficacy rating for each therapy program is based on a numerical rating scale of 1 to 5, whereby the efficacy rating of “1” indicates a relatively ineffective therapy program and an efficacy rating of “5” indicates a relatively efficacious therapy program. Efficacy may refer, in general, to a combination of complete or partial alleviation of symptoms alone, or alleviation of symptoms in combination with a degree of undesirable side effects.

In some cases, an overall evaluation metric 158 may be generated for each tested therapy program, where the specific evaluation metrics, such as the efficacy rating 154 and/or number of anxiety events 156, are weighted according to their relative importance to the therapy program evaluation. For example, the clinician may determine that the number of anxiety events 156 should have twice the weight as the efficacy rating, due to the subjective nature of the efficacy rating and the relatively objective nature of the number of detected anxiety events 156.

In the example shown in FIG. 11, the number of detected anxiety events 156 is categorized into groups that are each associated with a number on a scale of 1-5. For example, a score of “1” indicates a number of anxiety events between 21 or more, a score of “2” indicates a number of anxiety events between 16 and 20, a score of about “3” indicates a number of anxiety events between 11 and 15, a score of about “4” indicates a number of anxiety events between 6 and 10, and a score of about “5” indicates 0 to 5 events were detected during therapy delivery according to the respective therapy program.

The number of detected anxiety events 156 may then be combined with the efficacy rating to arrive at the overall metric. For example, with respect to Program A, the efficacy rating is 3 and the number of detected anxiety events 156 is 11, which indicates a score of 3. If the number of detected anxiety events 156 has twice the weight as the efficacy rating, the overall metric would equal approximately 4.5 (i.e., (score*2+efficacy rating)/2). The overall metric described herein is provided for purpose of example only. Other types of overall metrics may also be used to evaluate therapy programs 152 and may assign other weights to the efficacy rating 154 and the number of detected anxiety events 156.

Processor 60 may receive input from the clinician or another user selecting one of the evaluation metric types according to which to order the list of therapy programs. For example, display 78 may be a touch screen display, and the clinician may select efficacy rating box 154, anxiety events box 156 or overall metric box 158, and processor 60 may order the list of therapy programs according to evaluation metric associated with the selected text box. As another example, the clinician may interact with programmer 28 via control pad 132 select an evaluation metric for ordering the list of therapy programs.

Upon receive the evaluation metric selection from the clinician, processor 60 of programmer 28 may order the list of therapy programs 152 according to the evaluation metric. For example, in some cases, the clinician may wish to decrease the number of anxiety events experienced by patient 14, and, therefore, may select anxiety events box 156 as the evaluation metric for ordering therapy programs. Processor 60 may order the list of therapy programs 152 in an ascending or descending order in terms of the number of associated anxiety events. In this manner, the clinician may relatively quickly ascertain which therapy programs are associated with the highest number of anxiety events, thereby indicating the respective therapy programs are relatively inefficacious, or conversely which programs are associated with the lowest number of anxiety events, thereby indicating the respective therapy programs are relatively efficacious.

As another example, if the clinician selects efficacy rating box 154, upon receiving the evaluation metric selection from the clinician, processor 60 of programmer 28 may order the list of therapy programs 152 according to the evaluation metric. If, for example, efficacy rating 154 is selected as the evaluation metric, processor 60 may order the list of therapy programs 152 in an ascending or descending order in terms of how effective the therapy program was perceived to be by patient 14. In this manner, the clinician may relatively quickly ascertain which therapy programs are associated with the highest subjective efficacy rating. As another example, if anxiety events 156 is selected as the evaluation metric, processor 60 may orders the list of therapy programs 152 based on the evaluation metric.

Ordering the list of therapy programs according to a user-chosen criteria enables the clinician to quickly identify the therapy programs that exhibited the best efficacy in terms of reducing the number of detected anxiety events, as well as to identify the respective efficacy rating provided by patient 14 for the therapy programs. In contrast, without the automatic ordering of the therapy programs list according a user-chosen criteria, the clinician must typically manually sort through the data in order to identify the therapy program with the desired evaluation metric values.

Other evaluation metrics that processor 60 may determine and present to the user via GUI 150 include a power usage rating that indicates how much power IMD 16 consumes when generating and delivering therapy to patient 14 according to the associated therapy program.

During therapy delivery by IMD 16, IMD 16 may detect an anxiety episode based on a physiological signal and determine whether the anxiety episode is an anxiety event that is attributable to the anxiety disorder of patient 14 based on a patient activity signal indicative of a motor activity or voice activity characteristic of the anxiety event. As previously indicated, the detection of an anxiety event may be useful for controlling the delivery of therapy to patient 14. Rather than delivering therapy to patient 14 substantially continuously, IMD 16 may deliver therapy to patient 14 on an “on demand” basis, e.g., when an anxiety event is detected. In other examples, IMD 16 may deliver therapy to patient 14 substantially continuously but modify the therapy parameters when a certain number of anxiety events or anxiety episodes are detected within a predetermined duration of time.

FIG. 12 is a flow diagram of an example technique of delivering therapy to patient 14 based on a detected anxiety event. Processor 40 may receive a bioelectrical brain signal from sensing module 46 (FIG. 1) (160) and determine whether the bioelectrical brain signal is indicative of an anxiety episode (162). The techniques described above with respect to FIG. 4 may be used to detect the anxiety episode. For example, processor 40 can compare a peak, instantaneous, mean or median amplitude of the bioelectrical brain signal to a threshold value, compare a power level within one or more frequency bands of the bioelectrical brain signal to a threshold power level or comparing a ratio of power levels in two or more frequency bands of the bioelectrical brain signal to a threshold power level ratio, and detect the anxiety episode based on the comparison.

If processor 40 determines that the bioelectrical brain signal is not indicative of an anxiety episode (162), processor 40 may continue monitoring the bioelectrical brain signal until an anxiety episode is detected. Processor 40 receives patient activity information (164), such that upon detecting the anxiety episode based on the bioelectrical brain signal, processor 40 determine whether the patient activity information is indicative of a motor component or other activity of an anxiety event (166). In particular, processor 40 determines whether the motor component is detected during the detected anxiety episode. Processor 40 can receive the patient activity information substantially continuously or periodically from the patient activity sensor 36 (e.g., by receiving an electrical signal generated by motion sensor 36 that changes as a function of patient activity). In some examples, processor 40 receives patient activity information from sensor 36 prior to detection of the anxiety episode, e.g., at intervals that are unrelated to the detection of an anxiety episode. In other examples, processor 40 interrogates activity sensor 36 for patient activity information upon detection of the anxiety episode In either example, the activity information can be stored by memory 42 on a temporary or permanent basis.

The motor component may be specific to patient 14 and processor 40 may be configured to detect the specific behavioral or other motor activity that patient 14 engages during an anxiety event. As previously indicated, the motor component can be a predetermined patient activity associated with the anxiety event, such as an overt act, such as a compulsion (e.g., repetitive hand washing) or a non-overt act (e.g., incidental motor tics). As previously indicated, the activity component of an anxiety event can also include vocal activity, in which case, processor 40 may determine whether the voice activity component is detected instead of or in addition to the motor activity.

If the motor component of an anxiety event is not detected during the detected anxiety episode (166), processor 40 determines that the detected anxiety episode is not an anxiety event. Accordingly, processor 40 may not take any action to adjust therapy delivery to patient 14 and may continue to monitor the bioelectrical brain signal (160) until another anxiety episode is detected.

On the other hand, if the motor component of an anxiety event is detected during the detected anxiety episode (166), processor 40 determines that the detected anxiety episode is an anxiety event. Processor 40 may adjust therapy delivery (168) in response to detecting the anxiety event. In some cases, processor 40 initiates the delivery therapy to patient 14 to help mitigate the detected anxiety event. In this way, IMD 16 provides on demand therapy to patient 14 upon detecting a change in the mood state of patient 14, as indicated by the occurrence of the anxiety event.

In other cases, such as when IMD 16 is already delivering therapy to patient 14, processor 40 determines that the therapy parameter values currently implemented by IMD 16 to deliver therapy to patient 14 are insufficient because of the detection of the anxiety event. Thus, processor 40 may modify one or more of the therapy parameter values response to detecting the anxiety event. For example, processor 40 can select another therapy program from memory 42 (FIG. 2) or modify one or more particular therapy parameter values of a selected therapy program. The therapy parameter values may be modified if IMD 16 provides closed loop therapy to patient 14 (e.g., in response to a detected anxiety event) or if IMD 16 provides open loop therapy to patient 14 (e.g., independent of sensed patient parameters).

In some examples, processor 40 adjusts therapy delivery to patient 14 (168) only upon determining that a certain number of anxiety events were detected within a predetermined period of time. Processor 40 may implement a counter or any other technique for counting the number of anxiety events. For example, if processor 40 determines that over five anxiety events were detected in a 60 minute period of time, processor 40 may adjust therapy delivery to patient 14 (168). Other threshold number of anxiety events or periods of time may be used to determine when therapy delivery to patient 14 is adjusted.

Many anxiety episodes may occur and may not necessarily be attributable to the anxiety disorder of patient 14, but may instead be part of the ordinary thoughts of patient 14. In order to distinguish between which anxiety episodes merit therapy delivery or are undesirable, processor 40 of IMD 16, processor 60 of programmer 28 or another device may determine which of the many sensed anxiety episodes are anxiety events that are attributable to the anxiety disorder of patient 14. Anxiety events may be characterized by a behavioral change, such as engagement of a particular physical or mental activity by patient 14, which may manifest as a physical activity or a voice activity.

The technique shown in FIG. 12 controls therapy delivery to patient 14 upon the detection of an anxiety event. This type of therapy control results in a relatively high specificity of the therapy delivery because the anxiety events that are attributable to the anxiety disorder are used as triggers for therapy delivery. This may be beneficial in examples in which patient 14 experiences a high incidence of anxiety episodes that are not related to the anxiety disorder, or engage in repetitive or stereotypical behaviors that produce similar activity signals as the activity components of an anxiety event.

Other types of therapy delivery are contemplated. For example, rather than delivering therapy only when an anxiety event is detected as shown in FIG. 12, IMD 16 may deliver therapy to patient 14 upon detection of any combination of an anxiety episode, an anxiety event or an activity component that is typically associated with the anxiety event. While the therapy delivery upon detection of an anxiety episode may result in the delivery of therapy during anxiety episodes that are not anxiety events, the delivery of therapy upon detection of the anxiety episode may be useful for delivering therapy prior to the onset of any activity component (e.g., a compulsion or tic).

The technique described with respect to FIG. 12 includes monitoring a second patient parameter that is different than the first patient parameter in order to determine which of many sensed anxiety episodes are anxiety events for which therapy delivery by IMD 16 is implemented to help mitigate or prevent. In some examples described herein, the second patient parameter includes patient activity sensed via motion sensor 36. Processor 40 of IMD 16 or another device may analyze the output from motion sensor 36 to determine a patient activity associated with an anxiety episode. The patient activity may indicate, for example, whether patient 14 engaged in a known motor activity characteristic of the anxiety event. Examples of known motor activities include, but are not limited to, compulsive acts or motor tics.

In other examples, the second patient parameter includes voice activity sensed via motion sensor 36 or sensor 38, which can be a voice activity sensor, as described above. Sensor 38 may generate indicative of voice activity of patient 14 during an anxiety episode, which may indicate whether a vocal tic or other voice activity of patient 14 indicative of an anxiety event has occurred or is occurring. For example, a compulsion engaged in by patients with OCD during an anxiety event may be characterized by repeating of words, counting, praying or another detectable pattern of speech. The motor and voice activity may be monitored at the same time to detect the patient activity indicative of the anxiety event. As another example, if patient 14 with an anxiety disorder has a co-morbid tic disorder, such as Tourette's syndrome, the detection of a motor tic and vocal tic during an anxiety episode may be indicative of an anxiety event associated with Tourette's syndrome.

While FIG. 12 is described with respect to detecting an anxiety episode based on a bioelectrical brain signal, in other examples, other physiological signals in addition to or instead of a bioelectrical brain signal may be used to detect an anxiety episode. As described above, other physiological signals that indicate whether patient 14 is in an anxiety episode include, but are not limited to, ECG, facial EMG, transthoracic impedance, other signals that change as a function of heart rate or respiratory rate, or a signal indicative of thermal activity of skin of patient 14.

In addition, while the example technique shown in FIG. 12 is directed to controlling therapy delivery to patient 14 based on both a physiological parameter of patient 14 indicative of an occurrence of an anxiety episode and patient activity information that is indicative of a motor component (or voice activity component) of an anxiety event, in other examples, during therapy delivery by IMD 16, IMD 16 may detect an anxiety episode based on only the physiological signal and not the patient activity information. The patient activity information may be used as a secondary indicator of an anxiety event to determine one or more physiological signal characteristics (also referred to as signal signatures) that are indicative of anxiety events. Processor 40 of IMD 16 may then use the identified physiological signal characteristics to control therapy delivery to patient 14.

The second patient parameter that is an indicator of a specific patient activity attributable to the anxiety event is used to adjust an anxiety event algorithm implemented by IMD 16 to detect an anxiety event based on the first patient parameter, but not the second patient parameter. The second patient parameter is considered to be a relatively reliable indicator of an occurrence of an anxiety event because it is detects the results of an anxiety event while the specific patient activity associated with the anxiety disorder is actually occurring. These results may include, for example, physical patient activity or voice activity. In contrast to the second patient parameter, the first patient parameter monitors a patient parameter that indicates the anxiety event that occurs before the physical manifestations of the anxiety event. Thus, the first patient parameter is suitable for driving a course of action (e.g., therapy delivery or a warning) in order to either prevent the anxiety event or mitigate the severity of the anxiety event (e.g., by providing therapy to decrease the time in which patient 14 engages in a compulsion) or to provide a warning to the patient that an anxiety event is about to occur so that patient 14 is aware of the potential onset of the anxiety event.

Referring to examples in which the second patient parameter is patient activity, IMD 16 or programmer 28 may temporally correlate the patient activity information and a physiological signal (e.g., bioelectrical brain signals sensed via electrodes 24, 26) in order to determine the one or more physiological signal characteristics (also referred to as signal signatures) that are indicative of anxiety events. An anxiety event detection algorithm implemented by IMD 16 to detect the occurrence of an anxiety event based on the physiological signal may be adjusted or generated with the determined brain signal characteristics, such that IMD 16 is configured to detect the anxiety events based on the determined physiological signal characteristics. IMD 16 may learn the signal characteristics (i.e., the biomarkers) for the occurrence of the anxiety event such that IMD 16 may detect the anxiety event before it occurs and deliver therapy to help mitigate or even prevent the occurrence of the anxiety event.

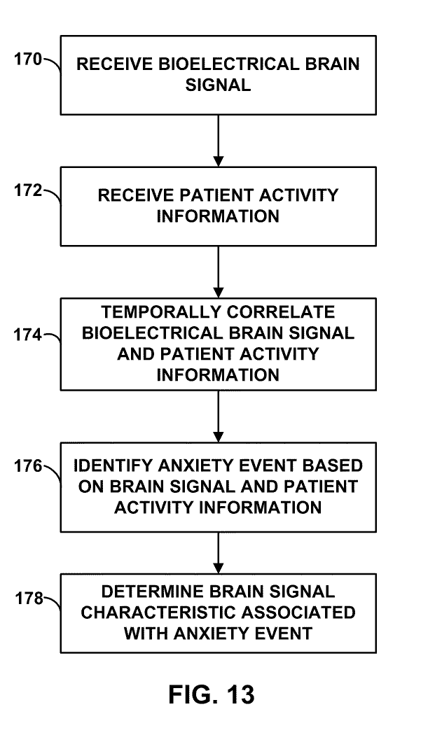

FIG. 13 illustrates an example technique processor 40 may implement to adjust an anxiety event detection algorithm. After receiving a bioelectrical brain signal from sensing module 46 (FIG. 2) (170) and receiving a patient activity signal from activity sensor 36 (FIG. 2) (172), processor 40 may temporally correlate the patient activity signal and the bioelectrical brain signal (174). For example, processor 40 can determine the patient activity signal that was generated by sensor 36 when sensing module 46 generated a particular segment of a bioelectrical brain signal.

Processor 40 may identify the occurrence of anxiety events based on the bioelectrical brain signal and patient activity signal (176), e.g., using the technique described with reference to FIG. 4, and, in some cases, based on patient input indicating the occurrence of an anxiety event. Processor 40 selects a portion of the bioelectrical brain signal temporally correlating to the occurrence of the patient activity component that is indicated by the patient activity signal and determines the bioelectrical brain signal characteristic indicative of the anxiety event identified based on the portion of the bioelectrical brain signal (178). In some examples, the characteristic comprises at least one of an amplitude threshold, a signal pattern, a power level within one or more frequency bands or a ratio of power levels in two or more frequency bands of the second signal. Processor 40 can store the signal characteristics in memory 42 of IMD 16 or a memory of another device.

The relevant portion of the bioelectrical brain signal that is correlated to the patient activity component may be, for example, the portion of the signal preceding the occurrence of the patient activity component of the anxiety event or a portion overlapping with the occurrence of the patient activity component. In some examples, processor 40 selects a bioelectrical brain signal characteristic that occurs prior to the onset of the anxiety event or one or more symptoms of the anxiety event (e.g., a vocal tic, motor tic or a compulsion), such that detection of the bioelectrical brain signal characteristic indicates the prospective occurrence of the anxiety event. This may help IMD 16 take some course of action in advance of the occurrence of the anxiety event, e.g., to mitigate the anxiety event, reduce the duration of the anxiety event or even prevent the anxiety event.

Initiating the course of action prior to the onset of the anxiety event may help prevent the occurrence of the anxiety event or at least mitigate the severity of any symptoms associated with the anxiety event. The bioelectrical brain signal characteristic may indicate that patient 14 is likely to have an anxiety event. Providing therapy prior to the onset of the anxiety event patient state may be more useful in some examples than providing therapy after the onset of the anxiety event, e.g., if the patient activity associated with the anxiety event (e.g., a compulsion, motor tic or vocal tic) is debilitating or distracting.

In some cases, the one or more brain signal characteristics indicative of an anxiety event include a threshold amplitude, whereby if the mean, median, instantaneous, highest or lowest amplitude of the bioelectrical brain signal during a predefined range of time is greater than or equal to the threshold amplitude, processor 40 determines that the anxiety event occurred or is about to occur. In other examples, the one or more bioelectrical brain signal characteristics include a pattern of the bioelectrical brain signal waveform, whereby if a sensed bioelectrical brain signal substantially correlates with a signal template representative of the pattern (e.g., the slope or timing between inflection points), processor 40 determines that the anxiety event occurred. Other types of brain signal characteristics that may be determined based on the bioelectrical brain signal corresponding to the patient activity signal that is indicative of the anxiety event are contemplated.

As previously indicated, processor 40 may also determine the one or more characteristics of the bioelectrical brain signal temporally correlating to the anxiety event based on patient input in addition to patient activity information. The patient input that indicates whether an anxiety event was experienced by patient 14 may provide an additional layer of information that helps generate a useful anxiety event detection algorithm. For example, in some cases, the patient activity signal generated by activity sensor 36 may indicate the occurrence of an anxiety event, but patient 14 may not have provided input indicating the occurrence of the anxiety event.

Processor 40 may determine that the anxiety event identified based on the patient activity signal is not an anxiety event that bothered patient 14, and, therefore, processor 40 may declassify the anxiety event. That is, if patient 14 fails to indicate that an anxiety event detected based on the patient activity signal occurred, processor 40 may not adjust the anxiety event detection algorithm to detect the similar anxiety events for subsequent therapy delivery. However, in some examples, patient 14 may not have provided input because of a lack of access to programmer 28 or because of an oversight. Thus, in some examples, processor 40 may determine and store the one or more bioelectrical brain signal characteristics associated with the anxiety event identified based on the patient activity signal despite the absence of patient input identifying the anxiety event. Processor 40 may ask patient 14 to provide immediate feedback about a detected event through programmer 28 using some sort of signaling mechanism described previously.

The technique shown in FIG. 13 may also be implemented by programmer 28 or a clinician, alone or with the aid of programmer 28, to generate an anxiety event detection algorithm that is used by IMD 16 for therapy delivery. While determining the one or more bioelectrical brain signal characteristics that are indicative of anxiety episodes may be useful for generating an anxiety event algorithm that provides useful anxiety disorder therapy to patient 14, the one or more characteristics of the bioelectrical brain signal that are indicative of an anxiety event may not be easily discernable. In some examples, programmer 28 or IMD 16 may automatically determine the one or more brain signal characteristics that are indicative of anxiety events, thereby eliminating the need for a clinician to manually review the relevant information. In addition, an automated technique for determining the one or more brain signal characteristics that are indicative of anxiety events may help reduce human error.

The technique shown in FIG. 13 may be implemented during programming of IMD 16, e.g., upon initial implantation of IMD 16 or during follow-up programming of IMD 16 after IMD 16 has delivered therapy to patient 14 for a while, to generate an anxiety event detection algorithm that discerns between anxiety episodes for which anxiety disorder therapy is not desirable and anxiety events for which therapy is desirable. Patient activity information provided by activity sensor 36 helps a clinician or programmer 28 determine the brain signal characteristics that are indicative of anxiety events without requiring long-term visual observation of patient 14. IMD 16 may collect the patient activity signal over a period of time (e.g., days or even weeks or more) to generate the information necessary to determine the brain signal characteristics that are indicative of anxiety events in accordance with the technique shown in FIG. 13.

In addition to generating an anxiety event detection algorithm based on the one or more brain signal characteristics, processor 40 may automatically update a stored anxiety event detection algorithm based on detection of anxiety events and the determination of the one or more brain signal (or other physiological signal) characteristics indicative of the detected anxiety event. Thus, in some examples, after processor 40 determines the bioelectrical brain signal characteristics associated with one or more anxiety events, processor 40 may adjust the anxiety event detection algorithm based on the determined bioelectrical brain signal characteristics. For example, processor 40 may store the determined bioelectrical brain signal characteristics as anxiety state information 56 (FIG. 2), such that the detection of the one or more determined bioelectrical brain signal characteristics in the future will result in detection of an anxiety event.

In some examples, IMD 16 relies on a primary patient parameter to control therapy delivery, thereby simplifying the anxiety event detection, but also employs a secondary parameter for determining whether the IMD 16 is properly detecting anxiety events. Relying on a primary patient parameter, rather than both the primary and secondary parameters, to control therapy delivery, may help reduce the complexity of the computations implemented by IMD 16 to detect an anxiety event, which may help minimize power consumption by IMD 16. IMD 16 can automatically identify anxiety events that have occurred despite therapy delivery based on the secondary patient parameter (e.g., patient activity level, patient posture or voice activity) that is not the parameter with which IMD 16 detects the anxiety episode to control therapy delivery. By determining the physiological signal (i.e., the primary patient parameter) characteristic that is associated with the anxiety events that have occurred, processor 40 of IMD 16 can automatically adjust the anxiety event detection algorithm implemented by IMD 16.

Adjusting the anxiety event detection algorithm implemented by IMD 16 with the brain signal characteristics that are known to be associated with anxiety events may help limit the number of false positive and false negative detections of anxiety events by IMD 16, and increase the specificity of anxiety event detection. For example, the patient activity information from motion sensor 36 (or voice activity sensor 38) may indicate whether IMD 16 failed to deliver therapy to patient 14 when an anxiety event occurred, thereby indicating that the anxiety event detection algorithm implemented by IMD 16 was not configured to detect the anxiety event. In addition, the patient activity information may indicate whether IMD 16 is delivering therapy to patient 14 when an anxiety episode is detected, but the anxiety episode is not an anxiety event, e.g., because of a lack of an associated motor component, thereby indicating the anxiety event detection algorithm is mischaracterizing some anxiety episodes as anxiety events.

The activity signal characteristics that indicate an activity component of an anxiety event of patient 14 and the physiological signal characteristics that indicate an anxiety episode of patient 14 may be determined using any suitable technique.

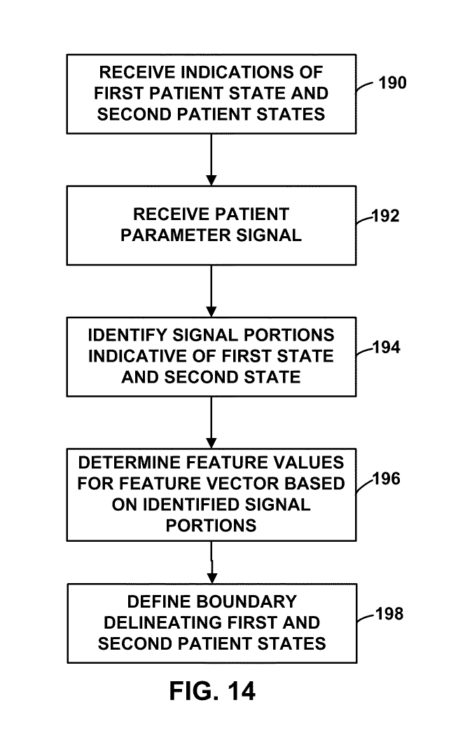

FIG. 14 is a flow diagram of an example technique for training a support vector machine (SVM) (also referred to as an SVM algorithm) or another supervised machine learning technique to respond to future patient parameter signal inputs and classify the patient parameter signal inputs as being representative of an anxiety event or a non-anxiety event, which may be an anxiety episode or a non-anxious mood state. Example supervised machine learning techniques include, but are not limited to, a genetic algorithm, an artificial neural network (e.g., based on a support vector machine (SVM), Bayesian classifiers, and the like) or other supervised machine learning techniques. Processor 60 of programmer 28 or another computing device (e.g., a medical data computing device implemented in a general purpose computer or a medical device), can implement the SVM algorithm to determine a classification boundary for determining whether a sensed patient parameter signal is indicative of a first patient state or a second patient state.

Processor 40, while implementing (or applying) the SVM algorithm receives a signal indicative of a patient parameter (e.g., a physiological parameter or a patient posture or activity level) and extracts signal characteristics directly from the signals or from a parameterized signal or data generated based on the raw patient parameter signal in order to generate the classification boundary. The signal characteristics are processed via the SVM algorithm in order to generate the classification boundary. In this way, processor 40 can implement an SVM algorithm or another supervised machine learning technique to generate a classification boundary based on training data (e.g., a patient parameter signal) from known occurrences of the patient state, where the classification boundary is used to predict or detect the occurrence of the patient state or evaluate the patient state, as described herein with respect to SVM-based algorithms.

In the example shown in FIG. 14, processor 40 generates a classification boundary that is used by IMD 16 or another device at a later time to determine whether a sensed patient parameter signal is indicative of a first patient state or a second patient state. In some examples, the first patient state is a state in which the anxiety event is present and the second patient state is a state in which the anxiety event is not present. In other examples, the first patient state is a state in which the anxiety episode is present and the second patient state in which the anxiety episode is not present. The technique shown in FIG. 14 may be performed by IMD 16, programmer 28 or another computing device. Thus, while processor 60 of programmer 28 is referred to throughout the description of FIG. 14, in other examples, any part of the technique shown in FIG. 14 may be implemented by processor 40 of IMD 16 (FIG. 2), another medical device (e.g., an external medical device) or another computing device.

In the technique shown in FIG. 14, a SVM-based patient state detection algorithm is trained to detect a patient state (e.g., an anxiety event state) of a specific patient based on values of features (e.g., signal characteristics) of one or more patient parameter signals known to be indicative of the patient state and features one or more patient parameter signals known to not be indicative of the patient state. The patient parameter signals may be, for example, the physiological signal (e.g., bioelectrical brain signal) indicative of an anxiety episode or a patient activity signal (e.g., generated by motion sensor 36 or voice activity sensor 38) indicative of a specific activity component of an anxiety event. The SVM determines a boundary that delineates the features indicative of the patient state and features not indicative of the patient state.

Once the SVM is trained based on the known patient state data, processor 40 of IMD 16, processor 60 of programmer 28 or a processor of another device implements a SVM-based algorithm that uses the classification boundary to determine whether patient 14 is in a state in which an anxiety event is present. In particular, processor 40 determines the side of the boundary on which a particular feature extracted from a sensed patient parameter signal lies to determine whether the anxiety event is detected. As noted above, the patient state detection may be used for various courses of action, such as controlling therapy delivery, generating a patient notification or evaluating a patient anxiety disorder.

In accordance with the technique shown in FIG. 14, processor 60 receives an indication of a first patient state (190), which may be, for example, an indication from patient 14 that an anxiety event is occurring or has occurred. In some examples, patient 14 provides input indicating the occurrence of the patient state via user interface 66 (FIG. 3) of programmer 28 or another user input mechanism, such as a device dedicated to receiving input from patient 14 indicative of the occurrence of the patient state. The dedicated device can be, for example, a key fob with a limited number of input buttons (e.g., one or two buttons), a consumer electronic device (e.g., a cell phone or a personal digital assistant) that is configured to record the patient inputs, or any other suitable device capable of receiving and storing patient input. Processor 60 may receive the input from the dedicated device through a wired (e.g., a cable) connection or via a wireless connection. IMD 16 may receive direct patient input in some examples. For example, patient 14 may tap the skin superior to IMD 16, and IMD 16 may include a motion sensor that is configured to sense a particular pattern of tapping, which is then characterized as patient input.

The indication of the first patient state may include a date and time stamp to indicate the time at which the first patient state was detected or the time at which patient 14 provided input indicating the occurrence of the first patient state. Depending upon the anxiety disorder with which patient 14 is diagnosed, patient 14 may be unable to provide input indicating the occurrence of the first patient state until after the onset of the first patient state, and even after the termination of the first patient state. Thus, programmer 28 may include features that permit patient 14 to modify the patient input, such as by modifying the date and time stamp associated with the patient input to be more accurate. In some examples, patient 14 may also provide input indicating the end of the patient state. Processor 60 may also automatically associate the patient input indicating the occurrence of the anxiety event with a particular time range, rather than a specific time.

Processor 60 also receives a signal indicative of a patient parameter (192). In some examples, processor 60 receives the signal from IMD 16 or a separate implanted or external sensor (e.g., sensor 38 in FIG. 2), either of which may generate a signal indicative of a physiological parameter (e.g., bioelectrical brain signals, heart rate, temperature, and the like) or a signal indicative of another patient parameter, such as patient activity, patient posture or voice activity. In some examples, processor 60 receives more than one signal indicative of a respective patient parameter.

In the examples described herein, processor 60 receives the signal from IMD 16. However, in other examples, processor 60 may receive the patient parameter signal from another sensing device instead or in addition to IMD 16. Moreover, in examples in which processor 40 of IMD 16 performs at least a part of the technique shown in FIG. 14, processor 40 may receive the signal from sensing module 46 (FIG. 2). In the example shown in FIG. 14, the signal is stored by IMD 16 or, and processor 60 receives the signal from IMD 16 or the sensing device via wireless communication techniques. In examples in which IMD 16 comprises an external device, processor 60 may receive the signal from IMD 16 via a wired (e.g., a cable) connection. Processor 60 can receive the signal indicative of the patient parameter from IMD 16 on a substantially continuous basis, on a regular, periodic basis or processor 60 may interrogate IMD 16 to retrieve the signal.

IMD 16 or the separate sensing device may sense the patient parameter on a continuous basis, a substantially periodic and scheduled basis, or in response to receiving patient input. For example, upon receiving patient input via programmer 28 or directly via IMD 16, IMD 16 may begin storing the signal indicative of the patient parameter, and, in some examples, may also store the portion of the signal preceding the receipt of the patient input for at least a predetermined amount of time. IMD 16 may include a loop recorder or another temporary recording module to store the patient parameter signal, from which processor 40 of IMD 16 may retrieve the portion of the signal preceding the receipt of the patient input for storage in memory 42.

After receiving the indications of the first patient state and the patient parameter signal (190, 192), processor 60, automatically or with the aid of a clinician, identifies portions of the patient parameter signal that are indicative of the first patient state (194). In some examples, processor 60 temporally correlates the patient parameter signal with the indications of the first patient state to determine which portions of the patient parameter signal were sensed during the first patient state. In addition, in some examples, processor 60 also identifies the portions of the patient parameter signal that temporally correlate with the time immediately preceding the onset of the patient state and immediately after the termination of the patient state. Processor 60 may identify the portion of the patient parameter signal indicative of the first patient state as the portion that corresponds to a predetermined range of time prior to the indication of the occurrence of the first patient state and a predetermined range of time after the occurrence of the patient state, if such information is known.

Processor 60 also identifies portions of the patient parameter signal that are not indicative of the first state, i.e., indicative of the second state (194). In general, the second state may be a specific patient state (e.g., an anxiety event) or may generally be a state that is not the first state (e.g., not a state in which the anxiety episode or the anxiety event is observed).

In other examples, processor 60 identifies the signal portions indicative of the first and second patient states (104) based on input from the clinician. The clinician may determine which segments of a sensed patient parameter signal are associated with the first patient state and input the information to processor 60.

After identifying the relevant portions of the patient parameter signal indicative of the first and second patient states (194), processor 60, automatically or with the aid of a clinician, determines feature vectors based on the identified portions of the patient parameter signal (196). A feature vector is a vector defined by two or more feature values indicative of a patient parameter signal characteristic (e.g., a morphology of the signal). In some examples, the features include the power level (also referred to as spectral energy) of the patient parameter signal in one or more frequency bands, an amplitude (e.g., the instantaneous, peak, mean or median amplitude) of the portion of the patient parameter signal or a subportion of the portion, other signal characteristics, or combinations thereof.

A feature vector can include any number of features of the identified portion of the patient parameter signal. For example, the feature vector can include two features, whereby a first feature is the power level in a first frequency band and the second feature is the power level in a second frequency band that is different than the first band (but may overlap with the first band). The features of the feature vectors may be selected to help distinguish between the different patient states. In some examples, a clinician selects the features by evaluating the signal portions indicative of the first and second patient states and determining which signal characteristics help distinguish between the patient states. In other examples, processor 60 automatically determines the features of the feature vectors.

It may be desirable to limit the number of features used by the SVM because of limitations of the sensing capabilities of IMD 16 or the power consumption limits of IMD 16. In other examples, the feature vector can include up to 16 or more features. For example, the feature vector can include the power level in ten separate frequency bands. If IMD 16 includes sixteen separate channels for sensing, each channel can be used to extract any number of features for a respective feature vector. For example, for each channel, the energy in each of 10 separate energy bands could be used define the respective feature vector.

Each feature in the feature vector corresponds to one dimension in the feature space that the SVM uses to classify data segments as being representative of the first patient state or a second patient state (e.g., a state that is generally different than the first patient state or a specific, known state). Each feature vector defines a point in a feature space which the SVM uses to classify data. In this way, each data point defined by a feature vector is a quantitative representation of the monitored feature values for a given time and each feature vector defines one data point in the feature space that is used to generate the classification boundary.

In some examples, processor 60 automatically determines the feature values that define the feature vectors, e.g., by automatically determining the values of each of the selected features for each of the identified signal portions. In other examples, a clinician or another person determines the feature vectors and inputs the determined feature values of the feature vectors into programmer 28 for automatic determination of the classification boundary.

In some examples, the signal portions on which each feature vector is determined has a predetermined duration of time. As a result, each feature vector represents the patient state for that predetermined duration of time. Accordingly, a single occurrence of a patient state that persists for a period of time that is longer than the duration of the signal portion used to determine a single feature vector may be associated with multiple feature vectors. In some examples, the signal segment used to determine a feature vector has a duration of about 0.5 seconds to about 5 seconds, although other time windows are contemplated.

Processor 60 can determine the features of the feature vector using any suitable technique. For example, if the features include power levels in respective frequency bands, IMD 16 or another sensing device may automatically extract power levels in specific frequency bands and transmit the power levels to processor 60 of programmer 28. In some examples, the sensor (e.g., sensing module 46 of IMD 16) may include an analog sensing circuit with an amplifier that uses limited power to monitor a frequency in which a desired physiological signal is generated. The frequency selective sensing circuit can include a chopper-stabilized superheterodyne instrumentation amplifier and a signal analysis unit, and may utilize a heterodyning, chopper-stabilized amplifier architecture to convert a selected frequency band of a physiological signal, such as a bioelectrical brain signal, to a baseband for analysis. The physiological signal may be analyzed in one or more selected frequency bands to determine one or more features as described herein.

Examples of various additional chopper amplifier circuits that may be suitable for or adapted to the techniques, circuits and devices of this disclosure are described in U.S. Pat. No. 7,385,443, which issued on Jun. 10, 2008, to Timothy J. Denison, entitled “Chopper Stabilized Instrumentation Amplifier,” the entire content of which is incorporated herein by reference. Examples of frequency selective monitors that may utilize a heterodyning, chopper-stabilized amplifier architecture are described in U.S. Patent Application Publication No. 2009/0082691 by Denison et al., entitled “FREQUENCY SELECTIVE MONITORING OF PHYSIOLOGICAL SIGN” and filed on Sep. 25, 2008, U.S. Provisional Application No. 60/975,372 to Denison et al., entitled “FREQUENCY SELECTIVE MONITORING OF PHYSIOLOGICAL SIGNALS,” and filed on Sep. 26, 2007, commonly-assigned U.S. Provisional Application No. 61/025,503 to Denison et al., entitled “FREQUENCY SELECTIVE MONITORING OF PHYSIOLOGICAL SIGNALS, and filed on Feb. 1, 2008, and commonly-assigned U.S. Provisional Application No. 61/083,381, entitled, “FREQUENCY SELECTIVE EEG SENSING CIRCUITRY,” and filed on Jul. 24, 2008. The entire contents of above-identified U.S. Patent Application Publication No. 2009/0082691 and U.S. Provisional Application Nos. 60/975,372, 61/025,503, and 61/083,381 are incorporated herein by reference.

Processor 60, automatically without user input or based on user input, determines the feature vectors for each of the identified signal portions (196). Thus, processor 60 determines feature vectors for both signal portions indicative of the first patient state and signal portion indicative of the second patient state. Thus, the feature vector values for both signal portions indicative of the first patient state and signal portion indicative of the second patient state are determined. In this way, the SVM algorithm implemented by processor 60 is trained to classify data based on known feature vectors that are associated with one of the first or second states.

After determining a plurality of feature vectors for the first and second patient states, processor 60 automatically determines a boundary that delineates the first and second patient states based on the plurality of determined feature vectors (198). In particular, the classification boundary is defined to separate feature values associated with known patient states such that the feature values for a first patient state are on one side of the boundary and feature values from the second patient state are on the other. In this way, processor 60 separates the determined feature values (which may be arranged into feature vectors) into two classes, whereby a first class corresponds to the occurrence of the first patient state and the second class corresponds to the occurrence of the second patient state. Processor 60 automatically determines the boundary to maximize separation between the first and second patient classes. The boundary may be linear or non-linear.

Additional details regarding supervised machine learning algorithms, including support vector machine-based algorithms, are described in U.S. patent application Ser. No. 12/694,042 by Carlson et al., which is entitled, “PATIENT STATE DETECTION BASED ON SUPPORT VECTOR MACHINE BASED ALGORITHM,” and was filed on Jan. 26, 2010, U.S. patent application Ser. No. 12/694,053 by Denison et al., which is entitled, “POSTURE STATE DETECTION,” and was filed on Jan. 26, 2010, U.S. patent application Ser. No. 12/694,044 by Carlson et al., which is entitled, “PATIENT STATE DETECTION BASED ON SUPERVISED MACHINE LEARNING BASED ALGORITHM,” and was filed on Jan. 26, 2010, and U.S. patent application Ser. No. 12/694,035 by Carlson et al., which is entitled, “PATIENT STATE DETECTION BASED ON SUPPORT VECTOR MACHINE BASED ALGORITHM,” and was filed on Jan. 26, 2010. U.S. patent application Ser. Nos. 12/694,042, 12/694,053, 12/694,044, and 12/694,035 are hereby incorporated by reference in their entireties.

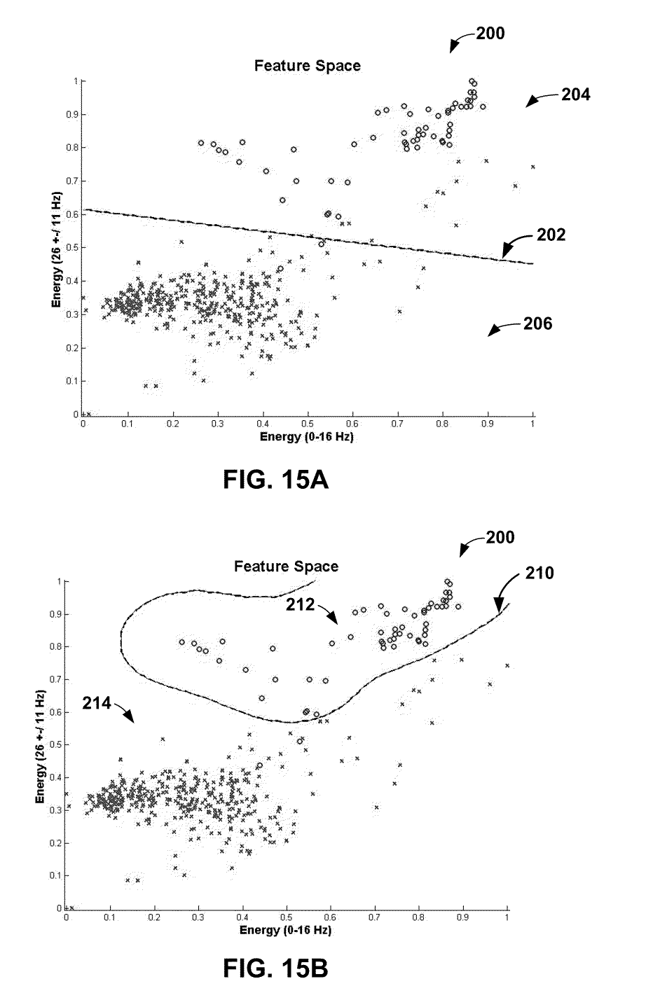

FIGS. 15A and 15B are conceptual illustrations of a features space 200 in which processor 60 has mapped plurality of feature vectors that correspond to an occurrence of a first patient state (e.g., an anxiety episode or an anxiety event) and a plurality of feature vectors that correspond to an occurrence of a second patient state (e.g., a non-anxious state or a state in which no anxiety event is detected, which may or may not be an anxiety episode). The feature space 200 shown in FIGS. 15A and 15B does not include feature vectors that are specific to an anxiety episode or an anxiety event. The feature space 200 is merely shown as an example of a feature space.

As previously indicated, each feature vector determined by processor 60 during a training phase based on patient parameter data associated with a known patient state defines a point in feature space 200 which the SVM algorithm uses to classify data. A two-dimensional feature space 200 is shown in FIGS. 15A and 15B. Because feature in a feature vector corresponds to one dimension in the feature space, the feature vectors that are mapped in feature space 200 include two features. In particular, the feature vectors shown in FIGS. 15A and 15B for purposes of illustration only include a first feature, which is an energy level within a first frequency band of about 0 Hz to about 16 Hz, and a second feature, which is an energy level within a second frequency band of about 15 Hz to about 37 Hz.

In FIG. 15A, processor 60 defined linear boundary 202 delineating the first and second patient states. In particular, linear boundary 202 defines first region 204 and second region 206 of feature space 200, which are later used by the SVM to classify a sensed patient state based on a sensed patient parameter signal. First region 204 is associated with the first patient class and second region 206 is associated with the second patient class. Processor 60 automatically determines linear boundary 130 to maximize separation between the first and second patient classes. Any suitable technique for determining linear boundary 130 may be used.

In FIG. 15B, processor 60 defined nonlinear boundary 210 delineating the first and second patient states. In particular, nonlinear boundary 210 separates feature space 200 into first region 212 associated with a first patient state and second region 214 associated with the second patient state. As with the linear boundary 202, processor 60 determines the boundary 210 that maximizes separation between the first and second patient classes. Processor 60 may determine nonlinear boundary 210 based on the training data points (determined based on the feature vectors associated with the known patient states) using any suitable technique. Processor 60 may, for example, use a kernel function to determine nonlinear boundary 210 that separates data points by patient state.

Techniques for generating linear and nonlinear classification boundaries are described in U.S. patent application Ser. No. 12/694,042 by Carlson et al., Ser. No. 12/694,053 by Denison et al., Ser. No. 12/694,044 by Carlson et al., and U.S. Ser. No. 12/694,035 by Carlson et al., which were previously incorporated by reference in their entireties.

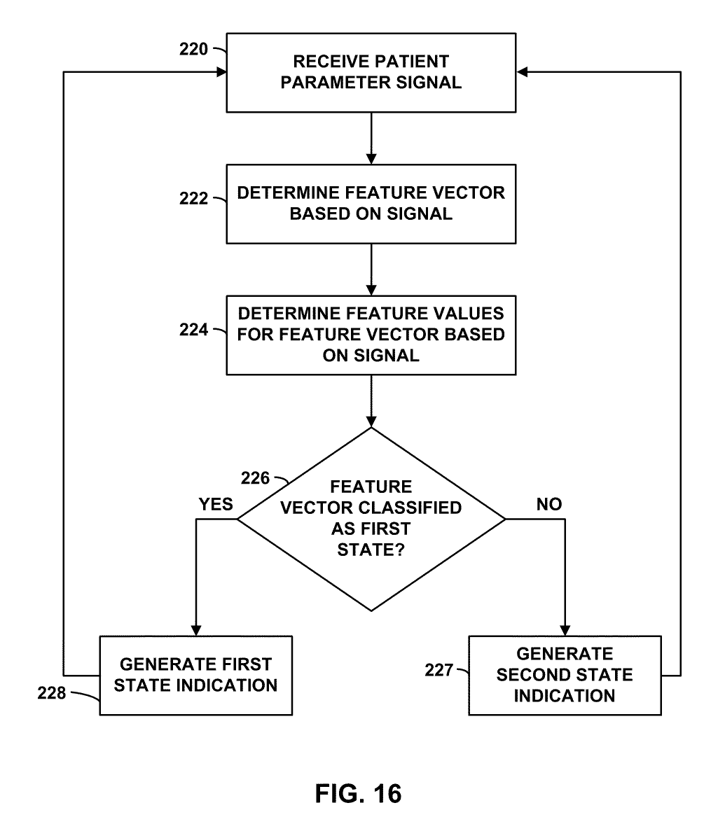

FIG. 16 is a flow diagram illustrating an example technique for detecting a patient state (e.g., an anxiety episode or an anxiety event) based on a real-time or stored patient parameter signal. The technique shown in FIG. 16 may be used with the technique shown in FIG. 4 to, for example, detect an anxiety episode (80) or determine whether a detected anxiety episode is associated with an activity component based on patient activity information (86). The technique shown in FIG. 16 may also be used with the technique shown in FIG. 10 to detect an anxiety event (142) or the technique shown in FIG. 12 to detect an anxiety episode (162) and/or detect a motor component (166) of an anxiety event.

FIG. 16 is described with respect to processor 40 of IMD 16. However, the technique shown in FIG. 16 may be performed by processor 40 of IMD 16, processor 60 of programmer 28, a processor of another device or any combination thereof.

In accordance with the technique shown in FIG. 16, processor 40 receives a signal indicative of a patient parameter (220). The signal can be, for example, a physiological signal or a signal indicative of patient activity level, patient posture or voice activity of patient 14. In some examples, the patient parameter signal that the SVM uses to determine the classification boundary is the same signal with which processor 40 determines the patient state. In some examples, the patient parameter signal is generated by sensing module 46 (FIG. 2), motion sensor 36, voice activity sensor 38, another sensor, or combinations thereof.

Processor 40 determines a feature values for determining a feature vector based on the signal (222). The features for which the values are determined are the same features with which the SVM algorithm generated the classification boundary, e.g., using the technique described in FIG. 14. Processor 40 can determine the feature vector values using any suitable technique, such as the technique described with respect to FIG. 14 for determining feature vectors for SVM training points. In some examples, processor 40 determines the feature vector based on a sample of the patient parameter signal having a predetermined duration of time. In this way, a plurality of determined feature vectors including respective feature values may represent the patient state for a known duration of time.

After determining the feature vector (222) based on the received signal, processor 40 compares the feature vector to a classification boundary (224), which may be linear (e.g., linear boundary 202 in FIG. 15A) or nonlinear (e.g., nonlinear boundary 210 in FIG. 15B). In particular, processor 40 maps the determined feature vector to the feature space and determines the side of the boundary in which the feature vector lies. If the feature vector does not lie within a side of the boundary associated with the first patient state, processor 40 may generate a second state indication (227) and then continue monitoring a physiological signal (220) and determining the feature vector (222). The second state indication may be, for example, a value, flag or signal that is stored in memory 42 of IMD 16 or another device (e.g., programmer 28). In some examples, processor 40 does not take any particular course of action upon generation of the second state indication (227), but merely stores the second state indication for later evaluation of the patient condition. However, in some examples, processor 40 may take an action in response to detecting the second state, such as adjusting therapy delivery (e.g., reducing an intensity of therapy delivery or deactivating therapy delivery). In other examples, processor 40 does not generate a second state indication, but merely continues monitoring a physiological signal (160) and determining the feature vector values (162) until the first state is detected.

If the feature vector lies within a side of the boundary associated with the first patient state processor classifies the determined feature vector in the feature space associated with the first state and determines that patient 14 is in the first state (226). Processor 40 may generate a first state indication (228). The first state indication may indicate the detection of an anxiety episode or an anxiety event. In some examples, the first state indication is at least one of a value, flag or signal that is stored in memory 42 of IMD 16 or another device (e.g., programmer 28). In some examples, processor 40 determines whether a predetermined number (e.g., four) of consecutive points are on one side of the boundary before determining patient 14 has changed states. A state change may be, for example, a change from an anxiety episode to a non-anxious mood state.

As previously indicated, determination of the patient state may be used for various purposes, such as to control therapy delivery (e.g., initiate, deactivate or modify therapy delivery), generate a patient notification, evaluate a patient condition or evaluate one or more therapy programs. Thus, upon generation of the first state indication (228), processor 40 of IMD 16 may take any suitable course of action.

Patient motion or posture, and, in some cases, voice activity of patient 14 may also be useful for detecting a mood state transition of patient 14, which may be useful for controlling therapy delivery to patient 14. For example, if patient 14 has manic depression, detection of a transition from a depressive state to a manic or hypomanic state (or vice versa) may be useful for controlling therapy delivery for managing manic depression. Manic depression can be characterized by the occurrence of one or more manic mood states or hypomanic mood states in which patient 14 has an abnormally elevated mood state or other symptoms of mania, and one or more depressive mood states, during which patient 14 has one or more symptoms of depression. One set of criteria for determining a manic episode, hypomanic episode or depressive mood states are defined by the Diagnostic and Statistical Manual of Mental Disorders (DSM-IV), which is a book, published by the American Psychiatric Association, which defines criteria used to diagnose various mental disorders, including depression.

As provided in the Diagnostic and Statistical Manual of Mental Disorders (DSM-IV), a manic mood state is characterized by a distinct period of abnormally and persistently elevated, expansive or irritable mood, lasting at least one week. In addition, the criteria for detecting a manic mood state include the presence of three or more of the following symptoms during the period of mood disturbance: (1) inflated self-esteem or grandiosity; (2) decreased need for sleep; (3) more talkative than usual or pressure to keep talking; (4) flight of ideas or subjective experience that thoughts are racing; (5) distractibility; (6) increase in goal-directed activity or psychomotor agitation; and (7) excessive involvement in pleasurable activities that have a high potential for painful consequences. The Diagnostic and Statistical Manual of Mental Disorders (DSM-IV) provides other criteria for diagnosing a manic episode. Any one or more of these symptoms of a manic mood state may be used to detect a manic mood state.

In addition, the Diagnostic and Statistical Manual of Mental Disorders (DSM-IV), provides criteria for a hypomanic mood state, which may indicate the presence of a psychiatric disorder. A hypomanic mood state may be characterized by the presence of a distinct period of persistently elevated, expansive or irritable mood, lasting throughout at least four days, and is clearly different from a typical nondepressed mood for the patient, as well as the presence of three or more of the following symptoms within the period (or four or more if the patient was in an irritable mood): (1) inflated self-esteem or grandiosity; (2) decreased need for sleep; (3) more talkative than usual or pressure to keep talking; (4) flight of ideas or subjective experience that thoughts are racing; (5) distractibility; (6) increase in goal-directed activity or psychomotor agitation; and (7) excessive involvement in pleasurable activities that have a high potential for painful consequences. A hypomanic mood state may be less severe than a manic mood state. These symptoms may also be used to diagnose a hypomanic mood state.

As provided in the Diagnostic and Statistical Manual of Mental Disorders (DSM-IV), criteria for detecting a depressive mood state include the presence of either depressed mood or anhedonia in addition to four other symptoms within the same two week period. The symptoms include, for example, (1) depressed mood for most of the day and nearly every day; (2) anhedonia (diminished interest or pleasure in all or almost all activities most of the day and nearly every day; (3) significant weight loss when not dieting or weight gain, or a decrease in appetite, (4) insomnia or hypersomnia nearly every day; (5) psychomotor agitation (e.g., pacing around a room, writing one's hands, or other unintentional and purposeless motions) or retardation (e.g., feeling slowed down) nearly every day; (6) fatigue or loss of energy nearly every day; (7) feelings of worthlessness or excessive or inappropriate guilty nearly every day; (8) diminished ability to think or concentrate, or indecisiveness nearly every day, and (9) recurrent thoughts of death or suicidal ideation without a specific plan, or a suicide attempt or specific plan. The Diagnostic and Statistical Manual of Mental Disorders (DSM-IV) provides other criteria for MDD. Any one or more of these symptoms of a MDD episode may be used to detect a depressive mood state.

Given the indications of a depressive mood state and a manic mood state (or hypomanic mood state) described above, IMD 16 or another device can determine whether patient 14 is in a depressive or manic mood state based on patient activity level or patient posture. For example, a patient activity level determined based on a signal generated by motion sensor 36 or another sensor may indicate whether patient 14 is exhibiting a relatively low level of activity when a higher activity level is expected (e.g., during the day when patient 14 is not expected to be sleeping) or compared to a baseline state when a depressive episode is known to not be occurring. As another example, a patient posture determined based on a signal generated by motion sensor 36 or another sensor may indicate whether patient 14 is in a lying down posture state for a certain percentage of time when patient 14 is not expected to be sleeping. In some examples, a support vector machine algorithm, e.g., the algorithm described above with respect to FIGS. 14-16 may be used to determine whether an activity signal from sensor 36 indicates patient 14 is in a depressive mood state.

Patient activity level or patient posture may also indicate whether patient 14 is in a manic or hypomanic mood state. For example, a patient activity level determined based on a signal generated by motion sensor 36 or another sensor may indicate whether patient 14 is exhibiting a relatively high level of activity compared to a baseline activity level. The baseline activity level may be, for example, determined when patient 14 is known to not be in a manic or hypomanic mood state, and, in some examples, when patient 14 is known to not be in a depressive mood state. A baseline activity level may be different at different times of day reflecting circadian patterns.

In some examples, voice activity of patient 14 may also indicate whether patient 14 is in a manic or a hypomanic mood state. For example, an increase in the voice activity level compared to a baseline level may indicate that patient 14 is speaking in a rapid and frenzied manner (e.g., pressured speech), which may be a characteristic of a manic or hypomanic mood state. Voice activity may be used in combination with patient activity level to determine whether patient 14 is in a manic or hypomanic mood state. In some examples, a support vector machine algorithm, e.g., the algorithm described above with respect to FIGS. 14-16 may be used to determine whether an activity signal from sensor 36 or voice activity signal from sensor 38 indicates patient 14 is in a manic mood state.

Different therapy parameter values may be more efficacious for the manic (or hypomanic) mood state than the depressive mood state. Similarly, different therapy parameter values may be more efficacious for the depressive mood state than the manic or hypomanic mood states. For example, electrical stimulation therapy delivered by IMD 16 according to a first therapy program to a target tissue site within brain 12 may help prevent or mitigate the intensity of symptoms or duration of a manic mood state, and stimulation delivery by IMD 16 according to a second therapy program to a target tissue site within brain 12 may help prevent or mitigate the intensity or duration of a depressive mood state. The exact therapy parameter values of the stimulation therapy, such as the amplitude or magnitude of the stimulation signals, the duration of each signal, the waveform of the stimuli (e.g., rectangular, sinusoidal or ramped signals), the frequency of the signals, and the like, may be specific for the particular target stimulation site (e.g., the region of the brain) involved as well as the particular patient.

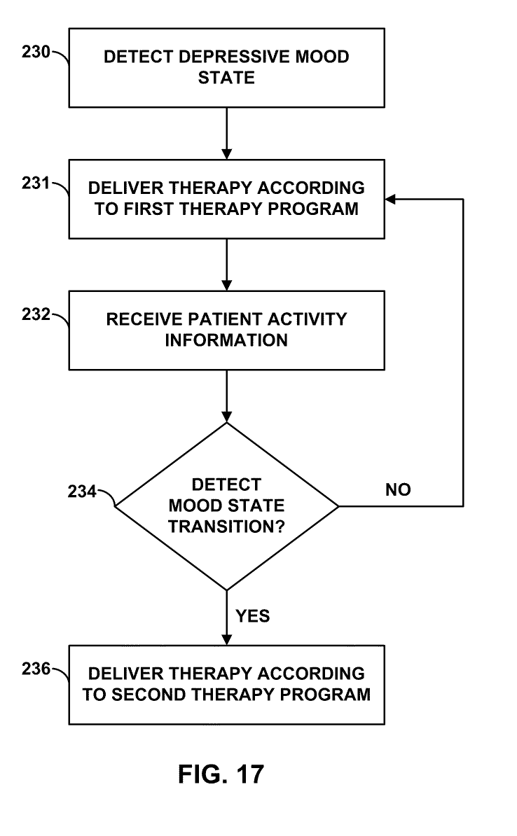

It may be desirable to control therapy delivery to patient 14 based on whether patient 14 is in a depressive mood state or a manic (or hypomanic) mood state. FIG. 17 is a flow diagram of an example technique for controlling therapy delivery to patient 14 based on a transition between a depressive mood state and a manic (or hypomanic) mood state that is detected based on patient activity (e.g., motor activity and/or voice activity). The example shown in FIG. 17 is directed to detecting a transition between a depressive mood state and a manic (or hypomanic) mood state. However, the technique shown in FIG. 17 may generally be used to detect a transition between first and second mood states that are associated with different activity levels and control therapy delivery based on the detected transition. In addition, while the technique shown in FIG. 17 is described as being performed by processor 40 of IMD 16, any one or more parts of the technique may be implemented by a processor of one of IMD 16, programmer 28, or another computing device, alone or in combination with each other.

Processor 40 of IMD 16 detects a depressive mood state (230) using any suitable technique. In some examples, processor 40 receives input from patient 14 indicating patient 14 is currently in a depressive mood state. Patient 14 may, for example, provide input via user interface 66 of programmer 28 (FIG. 3) or a user interface of another dedicated device or multipurpose device.

In other examples, processor 40 automatically detects the depressive mood state based on a bioelectrical brain signal or other physiological signal sensed by sensing module 46 of IMD 16 or another sensing device. For example, processor 40 may detect the depressive mood state by comparing a peak, instantaneous, mean or median amplitude of the bioelectrical brain signal to a threshold value, comparing a power level within one or more frequency bands of the bioelectrical brain signal to a threshold power level or comparing a ratio of power levels in two or more frequency bands of the bioelectrical brain signal to a threshold power level ratio, and detect the anxiety episode based on the comparison.

In addition, in other examples, processor 40 automatically detects the depressive mood state based on a signal generated by motion sensor 36 that indicates the relative activity level or the posture state of patient 14. For example, if patient 14 occupies a particular posture state (e.g., a lying down posture state) for a large percentage of time during a predetermined time range (e.g., when patient 14 is expected to be awake), processor 40 may determine that patient 14 is exhibiting symptoms of a depressive mood state, and, therefore, is in a depressive mood state. As another example, if the signal generated by motion sensor 36 indicates patient 14 is exhibiting a relatively low level of activity compared to a baseline state, processor 40 may determine that patient 14 is exhibiting symptoms of a depressive mood state, and, therefore, is in a depressive mood state. The baseline state may be, for example, an activity level of patient 14 when patient 14 is known to not be in a depressive mood state, and, in some examples, not in a manic mood state. In other examples, the baseline state may be indicated by a minimum activity level for more than one patient, where the minimum activity level is a level that a clinician deems to be indicative of a non-depressive mood state.

In addition or instead of the aforementioned techniques, processor 40 may implement a support vector machine algorithm to determine whether a sensed bioelectrical brain signal or other physiological signal indicates patient 14 is in a depressive mood state. Other techniques for detecting a depressive mood state are contemplated.

After detecting a depressive mood state (230), processor 40 controls stimulation generator 44 to deliver therapy delivery to patient 14 according to a first therapy program (231). The first therapy program may define a set of therapy parameter values that provide efficacious therapy to patient 14 for the depressive mood state. For example, delivery of therapy to patient 14 according to the first therapy program may help mitigate the severity or duration of a depressive mood state or even eliminate the depressive mood state.

Processor 40 may also receive patient activity information (232) that indicates activity of patient 14 that is affected by the depressive and manic (and hypomanic) mood states. For example, processor 40 may receive a signal from motion sensor 36 (FIG. 2) that generates a signal indicative of patient activity level or patient posture and/or a signal from sensor 38 (FIG. 1), which may generate a signal indicative of voice activity of patient 14. Based on this activity information, processor 40 may determine whether there is a mood state transition (234). The mood state transition may be, for example, a transition from the depressive mood state to a manic or hypomanic mood state.

In some examples, processor 40 detects the mood state transition by detecting a change in patient activity, which may be motor activity, a change in the percentage of time patient 14 occupies one or more specific posture states during a particular time range, and/or a change in voice activity. For examples, processor 40 may detect a mood state transition (234) by detecting an increase in patient activity level. Processor 40 may compare, for example, an amplitude or pattern of the patient activity signal generated by motion sensor 36 to a stored threshold or template to determine whether the patient activity level has increased. Processor 40 can also detect an increase or decrease in activity level of patient 14 between two periods of time by comparing a gross level of physical activity, e.g., activity counts based on footfalls or the like, undertaken by patient 14 during the respective periods of time. Processor 40 can determine activity counts using any suitable technique.

Suitable techniques for determining a patient's activity level or posture are described in U.S. Pat. No. 7,395,113 to Heruth et al., entitled, “COLLECTING ACTIVITY INFORMATION TO EVALUATE THERAPY,” and U.S. Patent Application Publication Serial No. 2008/0269812 by Gerber et al., entitled, “THERAPY ADJUSTMENT.” U.S. Pat. No. 7,395,113 and U.S. Patent Application Publication No. 2008/0269812 are incorporated herein by reference in their entireties. As described in U.S. Pat. No. 7,395,113, a processor may determine an activity level based on a signal from a sensor, such as an accelerometer, a bonded piezoelectric crystal, a mercury switch or a gyro, by sampling the signal and determining a number of activity counts during the sample period. For example, processor 40 may compare the sample of a signal generated by motion sensor 36 to one or more amplitude thresholds stored within memory 42. Processor 40 may identify each threshold crossing as an activity count. Where processor 40 compares the sample to multiple thresholds with varying amplitudes, processor 40 may identify crossing of higher amplitude thresholds as multiple activity counts. Using multiple thresholds to identify activity counts, processor 40 may be able to more accurately determine the extent of patient activity for both high impact, low frequency and low impact, high frequency activities, which may each be best managed by a different therapy program.

In other examples, processor 40 may detect a mood state transition (234) by detecting a specific motor activity known to be engaged in by patient 14 when patient 14 is in the manic mood state. The techniques described above with respect to detecting an activity component of an anxiety event may be used to detect the specific motor activity associated with a manic mood state.

Processor 40 can detect an increase in the percentage of time patient 14 spends in a posture state associated with the manic mood state (e.g., an upright and active posture state) or a decrease in the percentage of time patient 14 spends in a posture state associated with a depressive mood state (e.g., the lying down posture state) based on the output of motion sensor 36. Different characteristics of the signal generated by sensor 36 may be associated with specific posture states. The increase or decrease in the percentage of time spent in a particular posture state may be determined for the posture states observed during a predetermined sample of time, such as 30 minutes to one day or more.

In other examples, processor 40 may detect a mood state transition (234) by detecting an increase in voice activity of patient 14, which may indicated pressure speech, a characteristic of a manic or hypomanic mood state. The output from sensor 38 may indicate the relative voice activity level of patient 14. Thus, processor 40 can compare the output from sensor 38 (e.g., a mean or median amplitude) to a stored threshold value to determine whether the voice activity level of patient 14 has increased or otherwise indicates the manic or hypomanic mood state. Other techniques for detecting a mood state transition from the depressive mood state to the hypomanic or manic mood state based on patient activity are contemplated.

If processor 40 does not detect the mood state transition (234), processor 40 continues to deliver therapy according to the first therapy program that is appropriate to manage the depressive mood state of patient 14. On the other hand, if processor 40 detects the mood state transition (234) to a hypomanic or manic mood state, processor 40 may control stimulation generator 44 (FIG. 2) to generate and deliver therapy to patient 14 according to a second therapy program (236). The second therapy program defines at least one different therapy parameter value than the first therapy program. The second therapy program may define a set of therapy parameter values that provide efficacious therapy to patient 14 for the hypomanic or manic mood state. For example, delivery of therapy to patient 14 according to the first therapy program may help mitigate the severity or duration of a hypomanic or manic mood state or even eliminate the occurrence of the hypomanic or manic mood state.

In some examples, the disclosure is directed to a method comprising, with a medical device, delivering therapy to a patient to manage a first mood state of a patient, detecting a transition from the first mood state to a second mood state based on patient activity information, and adjusting therapy delivery by the medical device to the patient based on the transition. Adjusting therapy delivery to the patient based on the transition comprises modifying at least one stimulation parameter value with which the therapy is generated. The first mood state can comprise one of a depressive mood state, a manic mood state or a hypomanic mood state. In addition, the second mood state can comprise a different one of the depressive mood state, the manic mood state or the hypomanic mood state. In the method, the patient activity information is indicative of at least one of motion, posture state or voice activity of the patient.

In some examples, detecting the transition from the first mood state to the second mood state can comprise detecting a change in a motor activity level based on the patient activity information. Instead of or in addition to detecting a change in a motor activity level based on the patient activity information, detecting the transition from the first mood state to the second mood state comprises detecting a change in a voice activity level based on the patient activity information. In some examples, detecting the transition from the first mood state to the second mood state comprises detecting a change in a percentage of time the patient occupies a posture state over a predetermined duration of time based on the patient activity information. In the method

In some examples, the disclosure is directed to a system comprising a sensing module that generates a signal indicative of activity of a patient, a medical device that delivers therapy to the patient to manage a first mood state of the patient, and a processor that detects a transition from the first mood state to a second mood state based on patient activity information and controls the medical device to adjust the therapy delivery to the patient based on the transition. In some examples, the signal is indicative of at least one of motion, posture state or voice activity of the patient. In addition, the first mood state can comprise one of a depressive mood state, a manic mood state or a hypomanic mood state, and the second mood state can comprise a different one of the depressive mood state, the manic mood state or the hypomanic mood state. In the method, the patient activity information is indicative of at least one of motion, posture state or voice activity of the patient.

In some examples, the medical device of the system comprises the processor, while in other examples, the system comprises a medical device programmer that comprises the processor. The sensing module may, but need not be, physically separate from the medical device. In some examples, the medical device delivers at least one of electrical stimulation or a therapeutic agent. The processor can control the medical device to adjust therapy delivery to the patient based on the transition by modifying at least one stimulation parameter value according to which the medical device generates the therapy, initiating therapy delivery or even ceasing therapy delivery to the patient.

In some examples of the system, wherein the processor detects the transition from the first mood state to the second mood state by at least detecting change in a motor activity level based on the patient activity information. In addition or instead, the processor can detect the transition from the first mood state to the second mood state by at least detecting a change in a voice activity level based on the patient activity information. In some examples, the processor detects the transition from the first mood state to the second mood state by at least detecting a change in a percentage of time the patient occupies a posture state over a predetermined duration of time based on the patient activity information.

In some examples, the disclosure is directed to a system comprising means for delivering therapy to a patient to manage a first mood state of a patient, means for detecting a transition from the first mood state to a second mood state based on patient activity information, and means for adjusting therapy delivery to the patient based on the transition.

Moreover, in some examples, the disclosure is directed to an article of manufacture comprising a computer-readable storage medium comprising instructions that cause a programmable processor to control a medical device to deliver therapy to a patient to manage a first mood state of a patient, detect a transition from the first mood state to a second mood state based on patient activity information, and control the medical device to adjust therapy delivery to the patient based on the transition.

The techniques described in this disclosure, including those attributed to programmer 28, IMD 16, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. While the techniques described herein are primarily described as being performed by processor 40 of IMD 16 and/or processor 60 of programmer 28, any one or more parts of the techniques described herein may be implemented by a processor of one of IMD 16, programmer 28, or another computing device, alone or in combination with each other.

In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Various examples of the disclosure have been described. These and other examples are within the scope of the following claims.

1. A method comprising:
detecting, with a processor, an anxiety episode of a patient based on a physiological parameter of the patient; and
determining, with the processor, whether the anxiety episode is an anxiety event attributable to an anxiety disorder of the patient based on a signal indicative of patient activity.

2. The method of claim 1, wherein the signal is indicative of at least one of motion, posture state or voice activity of the patient.

3. The method of claim 1, wherein the anxiety disorder is characterized by the occurrence of a motor activity and determining whether the anxiety episode is the anxiety event comprises:
detecting, with the processor, the motor activity based on the signal; and
determining, with the processor, the anxiety episode is the anxiety event based on the detection of the motor activity.

4. The method of claim 3, wherein the motor activity comprises at least one of a compulsive motor activity, a motor tic, or at least one of a patient motion or posture state resulting from a compulsion.

5. The method of claim 1, wherein the anxiety disorder is characterized by the occurrence of a voice activity and determining whether the anxiety episode is the anxiety event comprises:
detecting, with the processor, the voice activity based on the signal; and
determining, with the processor, the anxiety episode is the anxiety event based on the detection of the voice activity.

6. The method of claim 5, wherein the voice activity comprises at least one of a compulsive act or a vocal tic.

7. The method of claim 1, wherein the signal comprises a first signal, and detecting the anxiety episode comprises:
receiving a second signal indicative of the physiological parameter; and
determining, with the processor, whether the second signal is indicative of the anxiety episode.

8. The method of claim 7, wherein determining whether the second signal is indicative of the anxiety episode comprises at least one of comparing a peak amplitude of the second signal to a threshold amplitude, comparing an average amplitude of the second signal to the threshold amplitude, comparing a median amplitude of the second signal to the threshold amplitude, comparing a trend in a waveform of the second signal over time to a template, comparing a power level within one or more frequency bands of the second signal to a threshold power level or comparing a ratio of power levels in two or more frequency bands of the second signal to a threshold power level ratio, and detecting the anxiety episode based on the comparison.

9. The method of claim 7, wherein the second signal is indicative of at least one of brain activity, a heart rate, respiratory rate, electrodermal activity, a facial expression or facial flushing of the patient.

10. The method of claim 7, wherein determining whether the second signal is indicative of the anxiety episode comprises determining whether the second signal is indicative of the anxiety episode with a support vector machine algorithm.

11. The method of claim 7, wherein determining whether the anxiety episode is an anxiety event attributable to an anxiety disorder of the patient based on the signal comprises:
temporally correlating the first and second signals; and
determining whether a portion of the first signal temporally correlated with the second signal is indicative of a predetermined patient activity.

12. The method of claim 7, further comprising determining a characteristic of the second signal indicative of the anxiety event at a first time, the method further comprising:
detecting the characteristic at a second time based on the second signal; and
adjusting therapy delivery to the patient upon detecting the characteristic.

13. The method of claim 1, further comprising adjusting therapy delivery to the patient upon determining the anxiety episode is the anxiety event attributable to the anxiety disorder.

14. The method of claim 1, further comprising generating a patient notification upon determining the anxiety episode is the anxiety event attributable to the anxiety disorder.

15. The method of claim 1, further comprising determining an anxiety metric associated with the anxiety episode, the anxiety metric comprising at least one of a duration of the anxiety episode, a latency between an anxiety episode onset and an onset of a patient activity indicative of the anxiety event, a classification of the latency, or a severity rating for the anxiety event.

16. The method of claim 1, further comprising:
at least one of determining a first amount of time the patient was in an anxiety episode during a selected time range, determining a second amount of time the patient was in an anxiety event during the selected time range, or determining a third amount of time the patient was an anxiety episode not attributable to the anxiety disorder; and
presenting the determined first, second or third amounts of times to a user via a user interface of a device.

17. The method of claim 1, further comprising:
associating the anxiety event with a therapy program implemented by a medical device at the time the anxiety event was detected, wherein the medical device delivers therapy to the patient according to therapy parameter values defined by the therapy program; and
ordering a list of therapy programs based on a number of associated anxiety events.

18. A method comprising:
receiving a first physiological signal indicative of an anxiety state of a patient;
receiving a second signal generated by a sensor, wherein the second signal is indicative of at least one of motion, posture state or voice activity of the patient; and
identifying, with a processor, an occurrence of an anxiety event during the anxiety state based on the first and second signals.

19. The method of claim 18, further comprising determining a characteristic of the first signal indicative of the anxiety event.

20. The method of claim 19, wherein the characteristic of the first signal indicative of the anxiety event prospectively indicates the anxiety event prior to occurrence of the anxiety event.

21. The method of claim 18, wherein identifying, with the processor, the occurrence of the anxiety event comprises:
detecting the anxiety state of the patient based on the first signal; and
during the anxiety state, detecting an activity component of the anxiety state based on the second signal, the activity comprising at least one of voice activity or motor activity.

22. A system comprising:
a first sensing module that generates a first signal indicative of physiological parameter of a patient;
a second sensing module that generates a second signal indicative of activity of the patient; and
a processor that detects an anxiety episode of the patient based on the first signal and determines whether the anxiety episode is an anxiety event attributable to an anxiety disorder of the patient based on the second signal.

23. The system of claim 22, wherein the second signal is indicative of at least one of motion, posture state or voice activity of the patient.

24. The system of claim 22, wherein the anxiety disorder is characterized by the occurrence a motor activity and the processor determines whether the anxiety episode is the anxiety event by at least detecting the motor activity based on the second signal and determining the anxiety episode is the anxiety event based on the detection of the motor activity.

25. The system of claim 24, wherein the motor activity comprises at least one of a compulsive motor activity, a motor tic, or at least one of a patient motion or posture state resulting from a compulsion.

26. The system of claim 22, wherein the anxiety disorder is characterized by the occurrence voice activity and the processor determines whether the anxiety episode is the anxiety event by at least detecting the voice activity based on the signal, and determining the anxiety episode is the anxiety event based on the detection of the voice activity.

27. The system of claim 22, wherein the processor determines whether the first signal is indicative of the anxiety episode by at least one of comparing a peak amplitude of the second signal to a threshold amplitude, comparing an average amplitude of the second signal to the threshold amplitude, comparing a median amplitude of the second signal to the threshold amplitude, comparing a trend in a waveform of the second signal over time to a template, comparing a power level within one or more frequency bands of the second signal to a threshold power level or comparing a ratio of power levels in two or more frequency bands of the second signal to a threshold power level ratio, and detecting the anxiety episode based on the comparison.

28. The system of claim 22, wherein the first signal comprises a bioelectrical brain signal or is indicative of at least one of a heart rate, respiratory rate, electrodermal activity, a facial expression or facial flushing of the patient.

29. The system of claim 22, wherein the processor implements a support vector machine algorithm to detect the anxiety episode based on the first signal.

30. The system of claim 22, wherein the processor detects an anxiety episode of the patient based on a first portion of the first signal and determines whether the anxiety episode is the anxiety event by at least temporally correlating the first and second signals and determining whether a second portion of the second signal temporally correlated with the first portion of the first signal is indicative of a predetermined patient activity.

31. The system of claim 22, wherein the processor determines a characteristic of the first signal indicative of the anxiety event.

32. The system of claim 31, further comprising a medical device, wherein the processor determines the characteristic of the first signal at a first time, detects the characteristic at a second time based on the first signal, and controls the medical device to adjust therapy delivery to the patient upon detecting the characteristic.

33. The system of claim 22, further comprising a medical device, wherein the processor controls the medical device to adjust therapy delivery to the patient upon determining the anxiety episode is the anxiety event attributable to the anxiety disorder.

34. The system of claim 22, wherein the processor generates a patient notification upon determining the anxiety episode is the anxiety event attributable to the anxiety disorder.

35. The system of claim 22, wherein the processor determines an anxiety metric associated with the anxiety episode, the anxiety metric comprising at least one of a duration of the anxiety episode, a latency between an anxiety episode onset and an onset of a patient activity indicative of the anxiety event, a classification of the latency, or a severity rating for the anxiety event.

36. The system of claim 22, further comprising a user interface, wherein the processor determines at least one of a first amount of time the patient was in an anxiety episode during a selected time range, a second amount of time the patient was in an anxiety event during the selected time range, or a third amount of time the patient was an anxiety episode not attributable to the anxiety disorder, and presents the determined first, second or third amounts of times to a user via the user interface.

37. The system of claim 22, further comprising a medical device, wherein the processor associates the anxiety event with a therapy program implemented by the medical device at the time the anxiety event was detected, wherein the medical device delivers therapy to the patient according to therapy parameter values defined by the therapy program and orders a list of therapy programs based on a number of associated anxiety events.

38. A system comprising:
a first sensing module that generates a first signal indicative of an anxiety episode of a patient;
a second sensing module that generates a second signal indicative of at least one of motion, posture state or voice activity of the patient; and
a processor that identifies an occurrence of an anxiety event based on the first and second signals.

39. The system of claim 38, wherein the processor determines the occurrence of the anxiety event by at least detecting the anxiety state of the patient based on the first signal, and, during the anxiety state, detecting an activity component of the anxiety state based on the second signal, the activity comprising at least one of voice activity or motor activity.

40. A system comprising:
means for generating a first signal indicative of a physiological parameter of a patient;
means for receiving a second signal generated by a patient activity sensor; and
means for determining whether a detected anxiety episode is an anxiety event attributable to an anxiety disorder of the patient based on the first and second signals.

41. The system of claim 40, wherein the second signal is indicative of at least one of motion, posture state or voice activity of the patient.

42. The system of claim 40, wherein the anxiety disorder is characterized by the occurrence a motor activity and the means for determining whether the detected anxiety episode is the anxiety event comprises:
means for detecting the motor activity based on the signal; and
means for determining the anxiety episode is the anxiety event based on the detection of the motor activity.

43. The system of claim 40, wherein the anxiety disorder is characterized by the occurrence voice activity and the means for determining whether the detected anxiety episode is the anxiety event comprises:
means for detecting the voice activity based on the signal; and
means for determining the anxiety episode is the anxiety event based on the detection of the voice activity.

44. A system comprising:
means for generating a first signal indicative of an anxiety state of a patient;
means for generating a second signal indicative of at least one of motion, posture state or voice activity of the patient; and
means for identifying an occurrence of an anxiety event during an anxiety state based on the first and second signals.

45. A computer-readable storage medium comprising instructions that cause a programmable processor to:
detect an anxiety episode of a patient based on a physiological parameter of the patient; and
determine whether the anxiety episode is an anxiety event attributable to an anxiety disorder of the patient based on a signal generated by a patient activity sensor.
